(19) United States
(12) Patent Application Publication
(10) Pub. No.: US 2022/0313670 A1
(43) Pub. Date: Oct. 6, 2022

(54) Methods Of Treating Fabry Disease In Patients Having Renal Impairment

(71) Applicant: Amicus Therapeutics, Inc., Philadelphia, PA (US)

(72) Inventor: Franklin Johnson, Bridgewater, NJ (US)

(73) Assignee: Amicus Therapeutics, Inc., Philadelphia, PA (US)

(21) Appl. No.: 17/618,277

(22) PCT Filed: Jun. 11, 2020
(86) PCT No.: PCT/US2020/037174

§ 371 Date: Dec. 10, 2021

Related U.S. Application Data
(60) Provisional application No. 62/859,904, filed on Jun. 11, 2019.

(51) Int. Cl. A61K 31/445

(52) U.S. Cl. CPC A61K 31/445; A61K 9/0053

(57) ABSTRACT

Provided are methods for treatment of Fabry disease in a patient having renal impairment. Certain methods comprise administering to the patient about 100 mg to about 300 mg free base equivalent of migalastat or salt thereof at a frequency of greater than once every other day, such as once every four or seven days. Certain methods comprise measuring lyso-Gb3 and/or migalastat in one or more plasma samples from the patient.

### TECHNICAL FIELD

Principles and embodiments of the present invention relate generally to the use of pharmacological chaperones for the treatment of Fabry disease, particularly in patients with varying degrees of renal impairment.

### BACKGROUND

Many human diseases result from mutations that cause changes in the amino acid sequence of a protein which reduce its stability and may prevent it from folding properly. Proteins generally fold in a specific region of the cell known as the endoplasmic reticulum, or ER. The cell has quality control mechanisms that ensure that proteins are folded into their correct three-dimensional shape before they can move from the ER to the appropriate destination in the cell, a process generally referred to as protein trafficking. Misfolded proteins are often eliminated by the quality control mechanisms after initially being retained in the ER. In certain instances, misfolded proteins can accumulate in the ER before being eliminated. The retention of misfolded proteins in the ER interrupts their proper trafficking, and the resulting reduced biological activity can lead to impaired cellular function and ultimately to disease. In addition, the accumulation of misfolded proteins in the ER may lead to various types of stress on cells, which may also contribute to cellular dysfunction and disease.

Such mutations can lead to lysosomal storage disorders (LSDs), which are characterized by deficiencies of lysosomal enzymes due to mutations in the genes encoding the lysosomal enzymes. The resultant disease causes the pathologic accumulation of substrates of those enzymes, which include lipids, carbohydrates, and polysaccharides. Although there are many different mutant genotypes associated with each LSD, many of the mutations are missense mutations which can lead to the production of a less stable enzyme. These less stable enzymes are sometimes prematurely degraded by the ER-associated degradation pathway. This results in the enzyme deficiency in the lysosome, and the pathologic accumulation of substrate. Such mutant enzymes are sometimes referred to in the pertinent art as “folding mutants” or “conformational mutants.”

Fabry Disease is a LSD caused by a mutation to the GLA gene, which encodes the enzyme α-galactosidase A (α-Gal A). α-Gal A is required for glycosphingolipid metabolism. The mutation causes the substrate globotriaosylceramide (Gb3, GL-3, or ceramide trihexoside) to accumulate in various tissues and organs. Males with Fabry disease are hemizygotes because the disease genes are encoded on the X chromosome. Fabry disease is estimated to affect 1 in 40,000 and 60,000 males, and occurs less frequently in females.

There have been several approaches to treatment of Fabry disease. One approved therapy for treating Fabry disease is enzyme replacement therapy (ERT), which typically involves intravenous, infusion of a purified form of the corresponding wild-type protein (Fabrazyme®, Genzyme Corp.). ERT has several drawbacks, however. One of the main complications with enzyme replacement therapy is rapid degradation of the infused protein, which leads to the need for numerous, costly high dose infusions. ERT has several additional caveats, such as difficulties with large-scale generation, purification, and storage of properly folded protein; obtaining glycosylated native protein; generation of an anti-protein immune response; and inability of protein to cross the blood-brain barrier to mitigate central nervous system pathologies (i.e., low bioavailability). In addition, replacement enzyme cannot penetrate the heart or kidney in sufficient amounts to reduce substrate accumulation in the renal podocytes or cardiac myocytes, which figure prominently in Fabry pathology.

Another approach to treating some enzyme deficiencies involves the use of small molecule inhibitors to reduce production of the natural substrate of deficient enzyme proteins, thereby ameliorating the pathology. This “substrate reduction” approach has been specifically described for a class of about 40 related enzyme disorders called lysosomal storage disorders that include glycosphingolipid storage disorders. The small molecule inhibitors proposed for use as therapy are specific for inhibiting the enzymes involved in synthesis of glycolipids, reducing the amount of cellular glycolipid that needs to be broken down by the deficient enzyme.

A third approach to treating Fabry disease has been treatment with what are called pharmacological chaperones (PCs). Such PCs include small molecule inhibitors of α-Gal A, which can bind to the α-Gal A to increase the stability of both mutant enzyme and the corresponding wild type.

One problem with current treatments is difficulty in treating patients exhibiting renal impairment, which is very common in Fabry patients and progresses with disease. On average, it take between about 10-20 years for patients to decline from normal kidney function to severe renal impairment, with some countries reporting even faster declines. By some estimates, about 10% of Fabry patients receiving ERT may have moderate renal impairment. Another 25% of males and 5% of females receiving ERT have an estimated glomerular filtration rate (eGFR) of less than 30, corresponding to severe kidney impairment or even renal failure. Of these, about half have severe kidney impairment, and about half are on dialysis.

Unfortunately, renal impairment will progress despite ERT treatment. A patient having an eGFR of 30 may deteriorate to the point of needing dialysis in two to five years. About 30% of patients receiving ERT will end up on dialysis or needing a kidney transplant, depending on the start of ERT. The earlier ERT is commenced, the longer renal function may be preserved, but commencement of ERT may be delayed because Fabry disease is rare and often misdiagnosed.

Further, and as discussed above, ERT often does not sufficiently penetrate the kidneys to reduce substrate accumulation, thereby allowing further damage during disease progression. With PC treatment, the kidneys are often how the drug is cleared from the body, and renal impairment may affect drug pharmacokinetics and/or drug pharmacodynamics. Thus, there is still a need for a treatment of Fabry patients who have renal impairment.

### SUMMARY

One aspect of the invention pertains to a method for treatment of Fabry disease in a patient having renal impairment, the method comprising administering to the patient about 100 mg to about 300 mg free base equivalent (FBE) of migalastat or salt thereof at a frequency of less than once every other day. In one or more embodiments, the patient has moderate renal impairment. In one or more embodiments, the patient has severe renal impairment. In some embodiments, the migalastat is in a solid dosage form. In one or more embodiments, the patient is administered about 123 mg FBE. In some embodiments, the patient is administered about 150 mg migalastat HCl. In one or more embodiments, the migalastat is administered orally. In one or more embodiments, the migalastat is administered for at least 28 days. In one or more embodiments, the migalastat is administered for at least 6 months. In one or more embodiments, the migalastat is administered for at least 12 months.

A second aspect of the invention pertains to a method for treatment of Fabry disease in a patient having renal impairment, the method comprising administering to the patient about 100 mg to about 300 mg FBE of migalastat or salt thereof at a frequency of once every seven days. In some embodiments, the patient has moderate renal impairment. In one or more embodiments, the patient has severe renal impairment. In some embodiments, the migalastat is in a solid dosage form. In one or more embodiments, the patient is administered about 123 mg FBE. In some embodiments, the patient is administered about 150 mg migalastat HCl. In one or more embodiments, the migalastat is administered orally. In one or more embodiments, the migalastat is administered for at least 28 days. In one or more embodiments, the migalastat is administered for at least 6 months. In one or more embodiments, the migalastat is administered for at least 12 months.

A third aspect of the invention pertains to a method for treatment of Fabry disease in a patient having renal impairment, the method comprising administering to the patient about 100 mg to about 300 mg FBE of migalastat or salt thereof at a first frequency of once every other day for a first time period; and administering to the patient about 100 mg to about 300 mg FBE of migalastat or salt thereof at a second frequency of less than every other day for a second time period. In one or more embodiments, the second frequency is in a range of once every three days to once every seven days. In one or more embodiments, the second frequency is once every four days. In one or more embodiments, the second frequency is once every seven days. In one or more embodiments, the administration at the second frequency begins after a reduction in the patient's eGFR. In one or more embodiments, the reduction in eGFR is from ≥30 mL/min/1.73 m² to ≤30 mL/min/1.73 m², i.e. a reduction in the patient's kidney function from mild or moderate renal impairment to severe renal impairment.

In some embodiments, the method further comprises:
(a) measuring lyso-Gb3 in one or more plasma samples from the patient;
(b) determining a first baseline lyso-Gb3 level during the first time period;
(c) measuring migalastat concentration, AUC_{0-∞} and/or C_{trough} in one or more plasma samples from the patient during the first time period; and
(d) beginning the administration at the second frequency after
   (i) an increase above the first baseline lyso-Gb3 level, and
   (ii) more than about 5 ng/mL of migalastat is measured 48 hours after administration of the migalastat during the first time period is measured, or there is a greater than 1.5-fold increase in AUC_{0-∞} and/or C_{trough} compared to normal renal function during the first time period.

In one or more embodiments, the method further comprises:
(a) measuring lyso-Gb3 in one or more plasma samples from the patient;
(b) determining a first baseline lyso-Gb3 level during the first time period;
(c) measuring migalastat concentration, AUC_{0-∞} and/or C_{trough} in one or more plasma samples from the patient during the first time period; and
(d) beginning the administration at the second frequency after
   (i) an increase above the first baseline lyso-Gb3 level, and
   (ii) more than about 5 ng/mL of migalastat is measured 48 hours after administration of the migalastat during the first time period is measured, or there is a greater than 1.5-fold increase in AUC_{0-∞} and/or C_{trough} compared to normal renal function during the first time period.

In some embodiments, the increase above the first baseline lyso-Gb3 level is at least about 30% and/or 2 nM. In one or more embodiments, measuring migalastat comprising measuring migalastat concentration, and administration at the second frequency begins after more than about 10 ng/mL of migalastat is measured 48 hours after administration of the migalastat during the first time period. In some embodiments, measuring migalastat comprises measuring AUC_{0-∞} or C_{trough}, and administration at the second frequency begins after there is a greater than 2-fold increase in AUC_{0-∞} and/or C_{trough} compared to normal renal function. In one or more embodiments, the second frequency is once every four days, and the method further comprises administering to the patient about 100 mg to about 300 mg FBE of migalastat or salt thereof at a third frequency of once every seven days for a third time period.

In some embodiments, the method further comprises: (a) measuring lyso-Gb3 in one or more plasma samples from the patient;
(b) determining a first baseline lyso-Gb3 level during first time period;
(c) measuring migalastat concentration, AUC_{0-∞} and/or C_{trough} in one or more plasma samples from the patient during the first time period;
(d) beginning the administration at the second frequency after
   (i) an increase above the first baseline lyso-Gb3 level, and
   (ii) more than about 5 ng/mL of migalastat is measured 96 hours after administration of the migalastat during the first time period is measured, or there is a greater than 1.5-fold increase in AUC_{0-∞} and/or C_{trough} compared to normal renal function during the first time period;
(e) determining a second baseline lyso-Gb3 level during the second time period; and
(f) beginning the administration at the third frequency after
   (i) an increase above the second baseline lyso-Gb3 level, and
   (ii) more than about 5 ng/mL of migalastat is measured 48 hours after administration of the migalastat during the second time period is measured, or there is a greater than 1.5-fold increase in AUC_{0-∞} and/or C_{trough} compared to normal renal function during the second time period.

In one or more embodiments, the increase above the first baseline lyso-Gb3 level is at least about 30% and/or 2 nM. In some embodiments, more than about 10 ng/mL of migalastat is measured 48 hours after administration of the migalastat during the first time period. In one or more embodiments, the second frequency is once every seven days.

In some embodiments, the method further comprises:
(a) measuring lyso-Gb3 in one or more plasma samples from the patient;
(b) determining a first baseline lyso-Gb3 level during the first time period;
(c) measuring migalastat concentration, AUC_{0-∞} and/or C_{trough} in one or more plasma samples from the patient during the first time period; and
(d) beginning the administration at the second frequency after
   (i) an increase above the first baseline lyso-Gb3 level, and
   (ii) more than about 5 ng/mL of migalastat is measured 48 hours after administration of the migalastat during the first time period is measured, or there is a greater than 1.5-fold increase in AUC_{0-∞} and/or C_{trough} compared to normal renal function during the first time period.

In one or more embodiments, the increase above the first baseline lyso-Gb3 level is at least about 30% and/or 2 nM. In some embodiments, more than about 10 ng/mL of migalastat is measured 48 hours after administration of the migalastat during the first time period.

In one or more embodiments, the patient has moderate renal impairment.

In some embodiments, the patient has severe renal impairment. In one or more embodiments, the migalastat is in a solid dosage form. In some embodiments, he patient is administered about 123 mg FBE. In one or more embodiments, the patient is administered about 150 mg migalastat HCl. In some embodiments, the migalastat is administered orally.

Another aspect of the invention pertains to the use of migalastat in the treatment of Fabry disease in a patient having renal impairment, wherein the migalastat is administered to a Fabry disease patient having renal impairment in an amount of about 100 mg to about 300 mg FBE of migalastat or salt thereof at a frequency of once every four or seven days. In one or more embodiments, the frequency is once every four days. In some embodiments, the frequency is once every seven days. In one or more embodiments, the patient has moderate renal impairment. In some embodiments, the patient has severe renal impairment. In one or more embodiments, the migalastat is in a solid dosage form. In some embodiments, the patient is administered about 123 mg FBE. In one or more embodiments, the patient is administered about 150 mg migalastat HCl. In some embodiments, the migalastat is administered orally.

Various embodiments are listed below. It will be understood that the embodiments listed below may be combined not only as listed below, but in other suitable combinations in accordance with the scope of the invention.

### BRIEF DESCRIPTION OF THE DRAWINGS

FIG. 1A shows the migalastat plasma concentrations of non-Fabry patients with varying degrees of renal impairment as a function of CL_{CR};

FIG. 1B shows the migalastat plasma concentrations of non-Fabry patients with varying degrees of renal impairment as a function of time post-dose;

FIG. 1C shows the migalastat area under the curve (AUC) of non-Fabry patients with varying degrees of renal impairment;

FIGS. 2A-D show migalastat concentration as a function of time for various dosing regimens and degrees of renal impairment;

FIGS. 3A-B show accumulation ratio and migalastat concentration for various dosing regimens;

FIG. 4 shows migalastat AUC_{0-∞} and migalastat concentration after 48 hours in non-Fabry patients with varying degrees of renal impairment as a function;

FIG. 5 shows plasma migalastat concentration after 48 hours as a function of eGFR_{MDRD} non-Fabry patients with varying degrees of renal impairment and two Fabry patients with renal impairment;

FIG. 6 shows plasma migalastat AUC_{0-∞} for non-Fabry patients with varying degrees of renal impairment and two Fabry patients with renal impairment;

FIGS. 7A-D show simulated median and observed migalastat concentration versus time in normal, severe, mild and moderate renal impairment subjects, respectively;

FIGS. 8A-D show migalastat Cₘₐₓ, AUC, Cₘᵢₙ and C₄₈ₕ, respectively, for normal, mild, moderate and severe renal impairment subjects;

FIGS. 9A-D show the steady state prediction for QOD for normal, severe, mild and moderate renal impairment subjects, respectively;

FIGS. 10A-D show migalastat Cₘₐₓ, AUC, Cₘᵢₙ and C₄₈ₕ, respectively, for normal, mild, moderate and severe renal impairment subjects;

FIG. 11A shows migalastat concentration after administration of 100 mg migalastat over 96 hours in a patient with moderate renal impairment;

FIG. 11B shows migalastat concentration after administration of 150 mg migalastat over 48 hours in a patient with normal kidney function;

FIGS. 12A-D show migalastat Cₘₐₓ, AUC, Cₘᵢₙ and C₄₈ₕ, respectively, for normal and moderate renal impairment subjects;

FIGS. 13A-E shows the full DNA sequence of human wild type GLA gene (SEQ ID NO: 1);

FIG. 14 shows the wild type GLA protein (SEQ ID NO: 2); and

FIG. 15 shows the lyso-Gb3 and eGFR of patient P3 over time.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Various aspects of the present invention pertain to particular dosing regimens of migalastat or a salt thereof for Fabry patients having renal impairment. Migalastat is a pharmacological chaperone used in the treatment of Fabry disease. This pharmacological chaperone is usually cleared from the body by the kidneys. However, patients who have renal impairment (a common problem for Fabry patients) may not be able to clear the migalastat from the body, and it was not previously known how patients with both Fabry disease and renal impairment would respond to migalastat therapy. Because pharmacological chaperones are also inhibitors, balancing the enzyme-enhancing and inhibitory effects of pharmacological chaperones such as migalastat is very difficult. Moreover, due to the complex interactions between Fabry disease and renal function and the lack of knowledge on the role of a pharmacological chaperone, migalastat dosing for Fabry patients with renal impairment is difficult to ascertain without significant clinical data and/or computer modeling.

Accordingly, one aspect of the invention pertains to a method for treatment of Fabry disease in a patient having renal impairment. In exemplary embodiments, the method comprises administering migalastat or a salt thereof every two, three, four, five, six or seven days. Although specific reference is made to administering every four or seven days, the methods and uses disclosed herein can also be used with other intermittent dosing regimens, such as every three, five or six days, based on, for example, the state of a patient's kidney.

In one or more embodiments, the method comprises administering to the patient about 100 mg to about 300 mg FBE of migalastat or salt thereof at a frequency of once every four days. In some embodiments, the method comprises administering to the patient about 100 mg to about 300 mg FBE of migalastat or salt thereof at a frequency of once every seven days. In some embodiments, the method comprises administering to the patient about 100 mg to about 300 mg FBE of migalastat or salt thereof at a frequency of once every four days for a first time period and then administering to the patient about 100 mg to about 300 mg FBE of migalastat or salt thereof at a frequency of once every seven days for a second time period. The patient may have mild, moderate or severe renal impairment.

Another aspect of the invention pertains to a use of migalastat in the treatment of Fabry disease in a patient having renal impairment, wherein the migalastat is administered to a Fabry disease patient having renal impairment in an amount of about 100 mg to about 300 mg FBE of migalastat or salt thereof at a frequency of once every four or seven days. The patient may have mild, moderate or severe renal impairment. In one or more embodiments, the patient has moderate or severe renal impairment. In specific embodiments, the patient has moderate renal impairment. In other specific embodiments, the patient has severe renal impairment.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolism due to deficient lysosomal α-galactosidase A activity. This defect causes accumulation of globotriaosylceramide (ceramide trihexoside) and related glycosphingolipids in vascular endothelial lysosomes of the heart, kidneys, skin, and other tissues.

The term “atypical Fabry disease” refers to patients with primarily cardiac manifestations of the α-Gal A deficiency, namely progressive globotriaosylceramide (GL-3) accumulation in myocardial cells that leads to significant enlargement of the heart, particularly the left ventricle.

A “carrier” is a female who has one X chromosome with a defective α-Gal A gene and one X chromosome with the normal gene and in whom X chromosome inactivation of the normal allele is present in one or more cell types. A carrier is often diagnosed with Fabry disease.

A “patient” refers to a subject who has been diagnosed with or is suspected of having a particular disease. The patient may be human or animal.

A “Fabry disease patient” refers to an individual who has been diagnosed with or suspected of having Fabry disease and has a mutated α-Gal A as defined further below. Characteristic markers of Fabry disease can occur in male hemizygotes and female carriers with the same prevalence, although females typically are less severely affected.

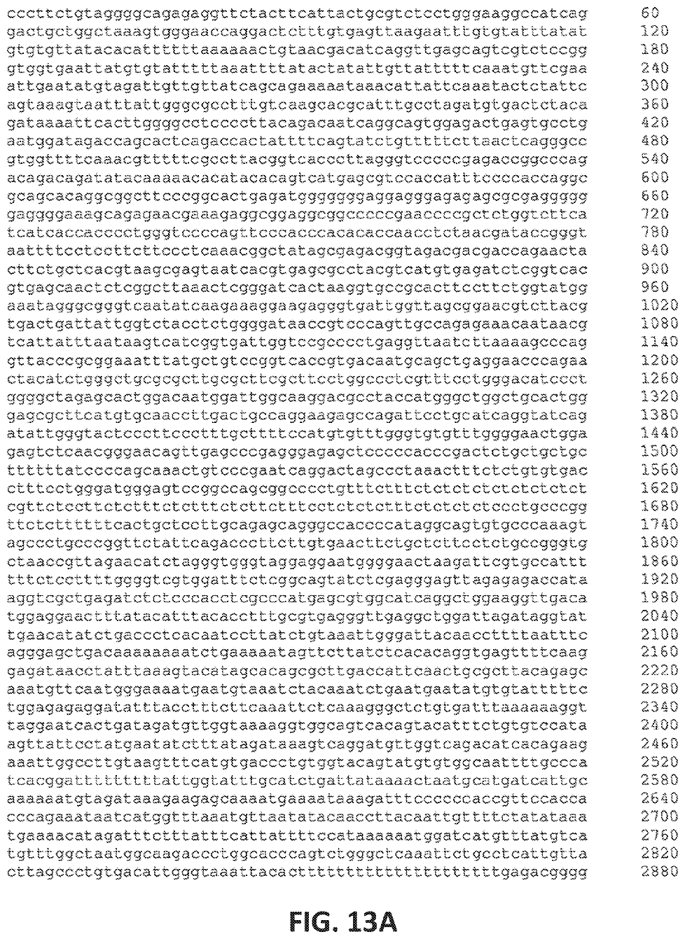

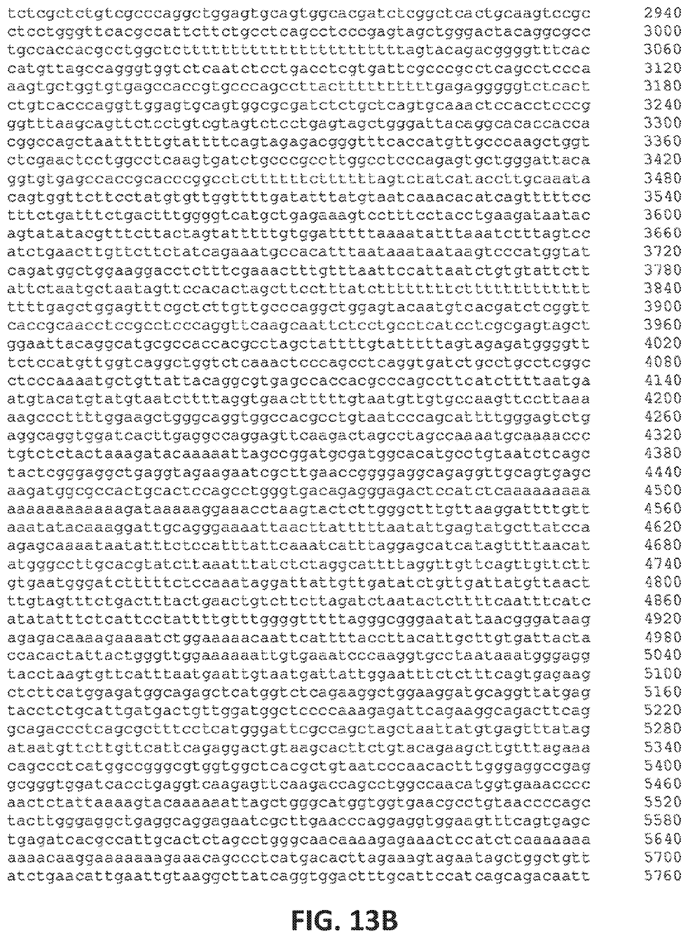

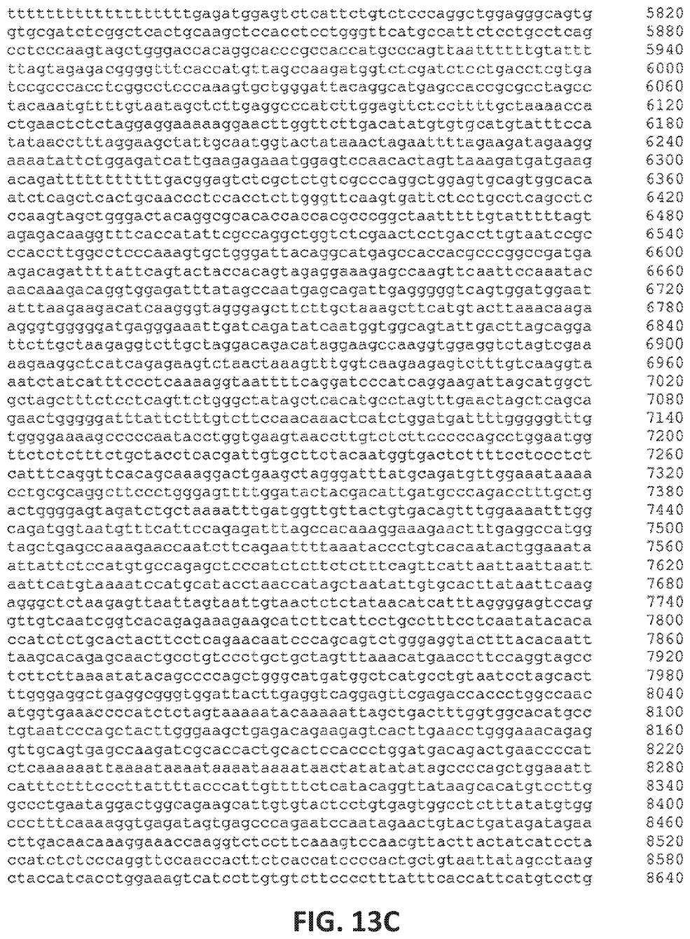

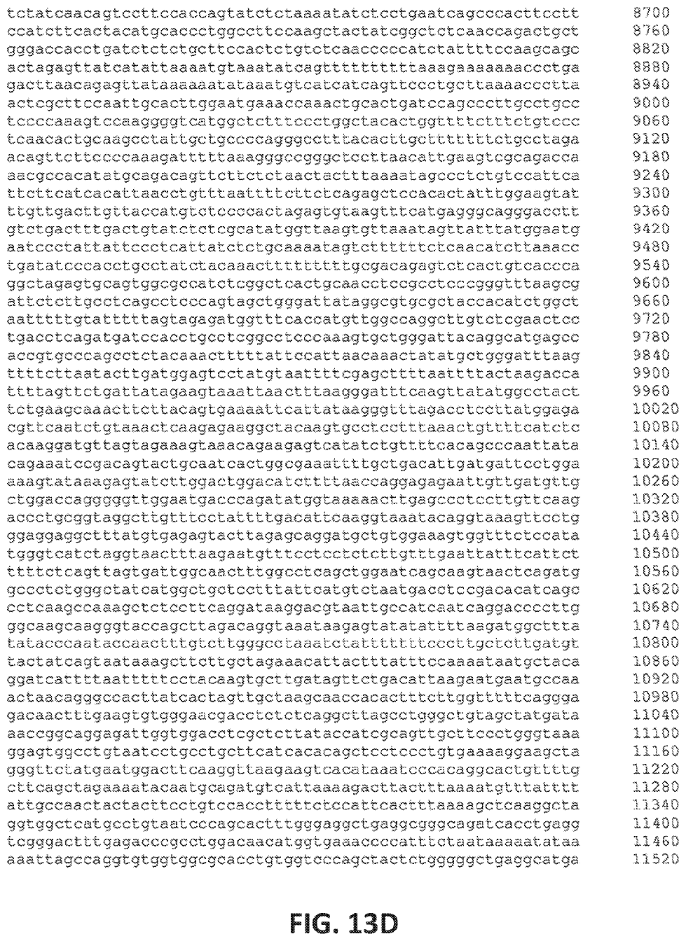

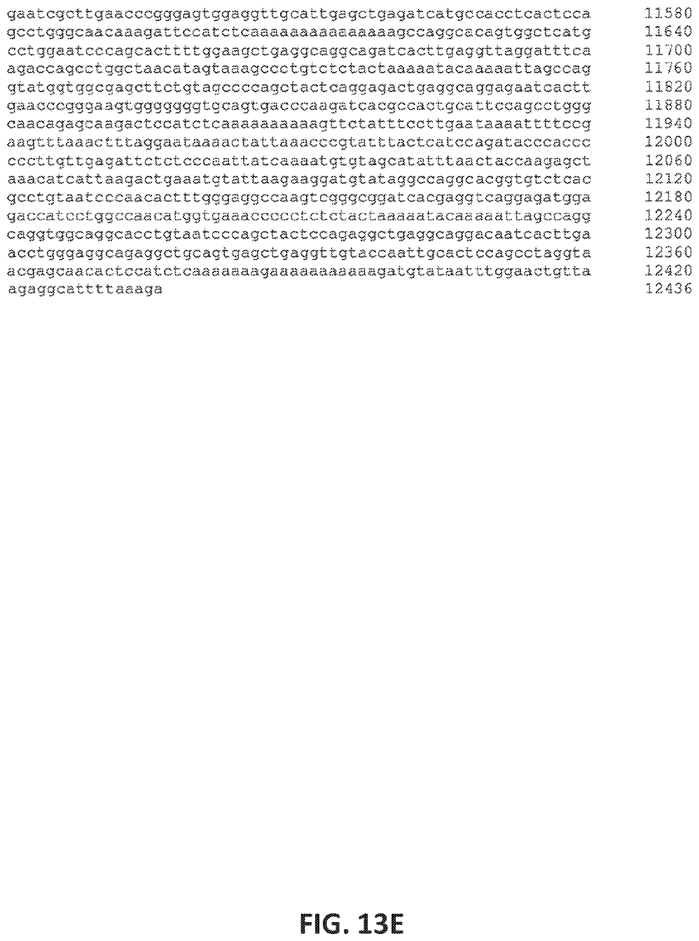

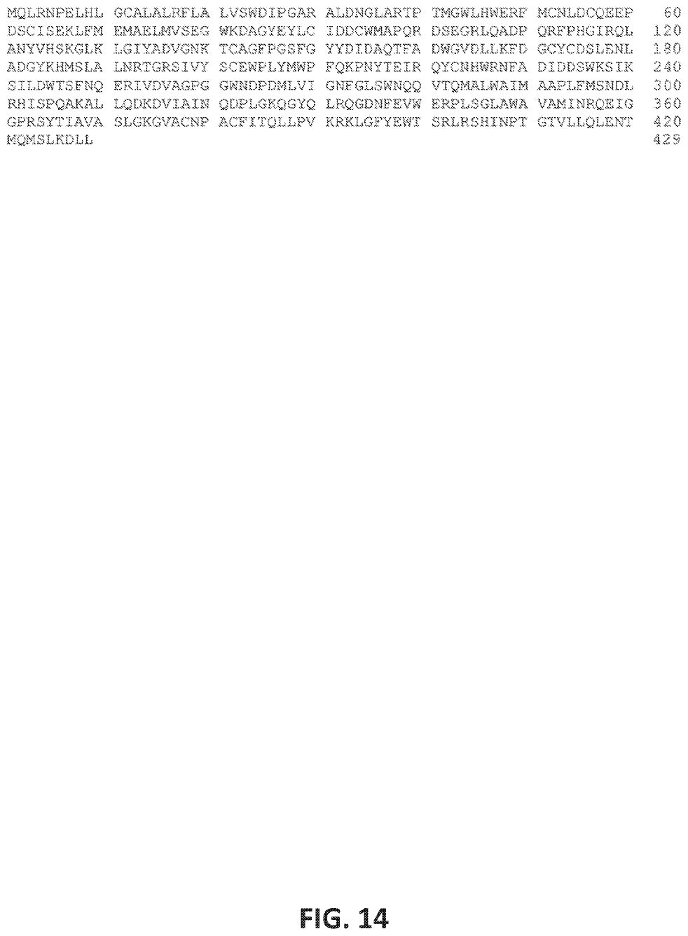

Human α-galactosidase A (α-Gal A) refers to an enzyme encoded by the human GLA gene. The full DNA sequence of α-Gal A, including introns and exons, is available in GenBank Accession No. X14448.1 and shown in SEQ ID NO: 1 and FIGS. 13A-E. The human α-Gal A enzyme consists of 429 amino acids and is available in GenBank Accession Nos. X14448.1 and U78027.1 and shown in SEQ ID NO: 2 and FIG. 14.

The term “mutant protein” includes a protein which has a mutation in the gene encoding the protein which results in the inability of the protein to achieve a stable conformation under the conditions normally present in the ER. The failure to achieve a stable conformation results in a substantial amount of the enzyme being degraded, rather than being transported to the lysosome. Such a mutation is sometimes called a “conformational mutant.” Such mutations include, but are not limited to, missense mutations, and in-frame small deletions and insertions.

As used herein in one embodiment, the term “mutant α-Gal A” includes an α-Gal A which has a mutation in the gene encoding α-Gal A which results in the inability of the enzyme to achieve a stable conformation under the conditions normally present in the ER. The failure to achieve a stable conformation results in a substantial amount of the enzyme being degraded, rather than being transported to the lysosome.

As used herein, the term “specific pharmacological chaperone” (“SPC”) or “pharmacological chaperone” (“PC”) refers to any molecule including a small molecule, protein, peptide, nucleic acid, carbohydrate, etc. that specifically binds to a protein and has one or more of the following effects: (i) enhances the formation of a stable molecular conformation of the protein; (ii) induces trafficking of the protein from the ER to another cellular location, preferably a native cellular location, i.e., prevents ER-associated degradation of the protein; (iii) prevents aggregation of misfolded proteins; and/or (iv) restores or enhances at least partial wild-type function and/or activity to the protein. A compound that specifically binds to e.g., α-Gal A, means that it binds to and exerts a chaperone effect on the enzyme and not a generic group of related or unrelated enzymes. More specifically, this term does not refer to endogenous chaperones, such as BiP, or to non-specific agents which have demonstrated non-specific chaperone activity against various proteins, such as glycerol, DMSO or deuterated water, i.e., chemical chaperones. In one or more embodiments of the present invention, the PC may be a reversible competitive inhibitor.

A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

As used herein, the term “specifically binds” refers to the interaction of a pharmacological chaperone with a protein such as α-Gal A, specifically, an interaction with amino acid residues of the protein that directly participate in contacting the pharmacological chaperone. A pharmacological chaperone specifically binds a target protein, e.g., α-Gal A, to exert a chaperone effect on the protein and not a generic group of related or unrelated proteins. The amino acid residues of a protein that interact with any given pharmacological chaperone may or may not be within the protein's “active site.” Specific binding can be evaluated through routine binding assays or through structural studies, e.g., co-crystallization, NMR, and the like. The active site for α-Gal A is the substrate binding site.

“Deficient α-Gal A activity” refers to α-Gal A activity in cells from a patient which is below the normal range as compared (using the same methods) to the activity in normal individuals not having or suspected of having Fabry or any other disease (especially a blood disease).

As used herein, the terms “enhance α-Gal A activity” or “increase α-Gal A activity” refer to increasing the amount of α-Gal A that adopts a stable conformation in a cell contacted with a pharmacological chaperone specific for the α-Gal A, relative to the amount in a cell (preferably of the same cell-type or the same cell, e.g., at an earlier time) not contacted with the pharmacological chaperone specific for the α-Gal A. This term also refers to increasing the trafficking of α-Gal A to the lysosome in a cell contacted with a pharmacological chaperone specific for the α-Gal A, relative to the trafficking of α-Gal A not contacted with the pharmacological chaperone specific for the protein. These terms refer to both wild-type and mutant α-Gal A. In one embodiment, the increase in the amount of α-Gal A in the cell is measured by measuring the hydrolysis of an artificial substrate in lysates from cells that have been treated with the PC. An increase in hydrolysis is indicative of increased α-Gal A activity.

The term “α-Gal A activity” refers to the normal physiological function of a wild-type α-Gal A in a cell. For example, α-Gal A activity includes hydrolysis of GL-3.

A “responder” is an individual diagnosed with or suspected of having a lysosomal storage disorder, such, for example Fabry disease, whose cells exhibit sufficiently increased α-Gal A activity, respectively, and/or amelioration of symptoms or improvement in surrogate markers, in response to contact with a PC. Non-limiting examples of improvements in surrogate markers for Fabry are lyso-Gb3 and those disclosed in US Patent Application Publication No. US 2010-0113517, which is hereby incorporated by reference in its entirety.

Non-limiting examples of improvements in surrogate markers for Fabry disease disclosed in US 2010/0113517 include increases in α-Gal A levels or activity in cells (e.g., fibroblasts) and tissue; reductions in of GL-3 accumulation; decreased plasma concentrations of homocysteine and vascular cell adhesion molecule-1 (VCAM-1); decreased GL-3 accumulation within myocardial cells and valvular fibrocytes; reduction in plasma globotriaosylsphingosine (lyso-Gb3); reduction in cardiac hypertrophy (especially of the left ventricle), amelioration of valvular insufficiency, and arrhythmias; amelioration of proteinuria; decreased urinary concentrations of lipids such as CTH, lactosylceramide, ceramide, and increased urinary concentrations of glucosylceramide and sphingomyelin; the absence of laminated inclusion bodies (Zebra bodies) in glomerular epithelial cells; improvements in renal function; mitigation of hypohidrosis; the absence of angiokeratomas; and improvements hearing abnormalities such as high frequency sensorineural hearing loss progressive hearing loss, sudden deafness, or tinnitus. Improvements in neurological symptoms include prevention of transient ischemic attack (TIA) or stroke; and amelioration of neuropathic pain manifesting itself as acroparaesthesia (burning or tingling in extremities). Another type of clinical marker that can be assessed for Fabry disease is the prevalence of deleterious cardiovascular manifestations. Common cardiac-related signs and symptoms of Fabry disease include left ventricular hypertrophy, valvular disease (especially mitral valve prolapse and/or regurgitation), premature coronary artery disease, angina, myocardial infarction, conduction abnormalities, arrhythmias, congestive heart failure.

The phrase “pharmaceutically acceptable” refers to molecular entities and compositions that are physiologically tolerable and do not typically produce untoward reactions when administered to a human. In some embodiments, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” in reference to a pharmaceutical carrier refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

The term “enzyme replacement therapy” or “ERT” refers to the introduction of a non-native, purified enzyme into an individual having a deficiency in such enzyme. The administered protein can be obtained from natural sources or by recombinant expression (as described in greater detail below). The term also refers to the introduction of a purified enzyme in an individual otherwise requiring or benefiting from administration of a purified enzyme, e.g., suffering from enzyme insufficiency. The introduced enzyme may be a purified, recombinant enzyme produced in vitro, or protein purified from isolated tissue or fluid, such as, e.g., placenta or animal milk, or from plants.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

As used herein, the term “free base equivalent” or “FBE” refers to the amount of migalastat present in the migalastat or salt thereof. In other words, the term “FBE” means either an amount of migalastat free base, or the equivalent amount of migalastat free base that is provided by a salt of migalastat. For example, due to the weight of the hydrochloride salt, 150 mg of migalastat hydrochloride only provides as much migalastat as 123 mg of the free base form of migalastat. Other salts are expected to have different conversion factors, depending on the molecular weight of the salt.

The term “migalastat” encompasses migalastat free base or a pharmaceutically acceptable salt thereof (e.g., migalastat HCl), unless specifically indicated to the contrary.

Fabry Disease

Fabry disease is a rare, progressive and devastating X-linked lysosomal storage disorder. Mutations in the GLA gene result in a deficiency of the lysosomal enzyme, α-Gal A, which is required for glycosphingolipid metabolism. Beginning early in life, the reduction in α-Gal A activity results in an accumulation of glycosphingolipids, including GL-3 and plasma lyso-Gb3, and leads to the symptoms and life-limiting sequelae of Fabry disease, including pain, gastrointestinal symptoms, renal failure, cardiomyopathy, cerebrovascular events, and early mortality. Early initiation of therapy and lifelong treatment provide an opportunity to slow disease progression and prolong life expectancy.

Fabry disease encompasses a spectrum of disease severity and age of onset, although it has traditionally been divided into 2 main phenotypes, “classic” and “late-onset”. The classic phenotype has been ascribed primarily to males with undetectable to low α-Gal A activity and earlier onset of renal, cardiac and/or cerebrovascular manifestations. The late-onset phenotype has been ascribed primarily to males with higher residual α-Gal A activity and later onset of these disease manifestations. Heterozygous female carriers typically express the late-onset phenotype but depending on the pattern of X-chromosome inactivation may also display the classic phenotype.

More than 800 Fabry disease-causing GLA mutations have been identified. Approximately 60% are missense mutations, resulting in single amino acid substitutions in the α-Gal A enzyme. Missense GLA mutations often result in the production of abnormally folded and unstable forms of α-Gal A and the majority are associated with the classic phenotype. Normal cellular quality control mechanisms in the endoplasmic reticulum block the transit of these abnormal proteins to lysosomes and target them for premature degradation and elimination. Many missense mutant forms are targets for migalastat, an α-Gal A-specific pharmacological chaperone.

The clinical manifestations of Fabry disease span a broad spectrum of severity and roughly correlate with a patient's residual α-GAL levels. The majority of currently treated patients are referred to as classic Fabry disease patients, most of whom are males. These patients experience disease of various organs, including the kidneys, heart and brain, with disease symptoms first appearing in adolescence and typically progressing in severity until death in the fourth or fifth decade of life. A number of recent studies suggest that there are a large number of undiagnosed males and females that have a range of Fabry disease symptoms, such as impaired cardiac or renal function and strokes, that usually first appear in adulthood. Individuals with this type of Fabry disease, referred to as later-onset Fabry disease, tend to have higher residual α-GAL levels than classic Fabry disease patients. Individuals with later-onset Fabry disease typically first experience disease symptoms in adulthood, and often have disease symptoms focused on a single organ, such as enlargement of the left ventricle or progressive kidney failure. In addition, later-onset Fabry disease may also present in the form of strokes of unknown cause.

Fabry patients have progressive kidney impairment, and untreated patients exhibit end-stage renal impairment by the fifth decade of life. Deficiency in α-Gal A activity leads to accumulation of globotriaosylceramide (Gb3) and related glycosphingolipids in many cell types including cells in the kidney. Gb3 accumulates in podocytes, epithelial cells and the tubular cells of the distal tubule and loop of Henle. Impairment in kidney function can manifest as proteinuria and reduced glomerular filtration rate.

Because Fabry disease can cause progressive worsening in renal function, it is important to understand the pharmacokinetics (PK) of potential therapeutic agents in individuals with renal impairment and particularly so for therapeutic agents that are predominantly cleared by renal excretion. Impairment of renal function may lead to accumulation of the therapeutic agent to levels that become toxic.

Because Fabry disease is rare, involves multiple organs, has a wide age range of onset, and is heterogeneous, proper diagnosis is a challenge. Awareness is low among health care professionals and misdiagnoses are frequent. Diagnosis of Fabry disease is most often confirmed on the basis of decreased α-Gal A activity in plasma or peripheral leukocytes (WBCs) once a patient is symptomatic, coupled with mutational analysis. In females, diagnosis is even more challenging since the enzymatic identification of carrier females is less reliable due to random X-chromosomal inactivation in some cells of carriers. For example, some obligate carriers (daughters of classically affected males) have α-Gal A enzyme activities ranging from normal to very low activities. Since carriers can have normal α-Gal A enzyme activity in leukocytes, only the identification of an α-Gal A mutation by genetic testing provides precise carrier identification and/or diagnosis.

Mutant forms of α-galactosidase A are considered to be amenable to migalastat are defined as showing a relative increase (+10 μM migalastat) of ≥1.20-fold and an absolute increase (+10 μM migalastat) of ≥3.0% wild-type (WT) when the mutant form of α-galactosidase A is expressed in HEK-293 cells (referred to as the “HEK assay”) according to Good Laboratory Practice (GLP)-validated in vitro assay (GLP HEK or Migalastat Amenability Assay). Such mutations are also referred to herein as “HEK assay amenable” mutations.

Previous screening methods have been provided that assess enzyme enhancement prior to the initiation of treatment. For example, an assay using HEK-293 cells has been utilized in clinical trials to predict whether a given mutation will be responsive to pharmacological chaperone (e.g., migalastat) treatment. In this assay, cDNA constructs are created. The corresponding α-Gal A mutant forms are transiently expressed in HEK-293 cells. Cells are then incubated ±migalastat (17 nM to 1 mM) for 4 to 5 days. After, α-Gal A levels are measured in cell lysates using a synthetic fluorogenic substrate (4-MU-α-Gal) or by western blot. This has been done for known disease-causing missense or small in-frame insertion/deletion mutations. Mutations that have previously been identified as responsive to a PC (e.g. migalastat) using these methods are listed in U.S. Pat. No. 8,592,362, which is hereby incorporated by reference in its entirety.

Pharmacological Chaperones

The binding of small molecule inhibitors of enzymes associated with LSDs can increase the stability of both mutant enzyme and the corresponding wild-type enzyme (see U.S. Pat. Nos. 6,274,597; 6,583,158; 6,589,964; 6,599,919; 6,916,829, and 7,141,582 all incorporated herein by reference). In particular, administration of small molecule derivatives of glucose and galactose, which are specific, selective competitive inhibitors for several target lysosomal enzymes, effectively increased the stability of the enzymes in cells in vitro and, thus, increased trafficking of the enzymes to the lysosome. Thus, by increasing the amount of enzyme in the lysosome, hydrolysis of the enzyme substrates is expected to increase. The original theory behind this strategy was as follows: since the mutant enzyme protein is unstable in the ER (Ishii et al., Biochem. Biophys. Res. Comm. 1996; 220: 812-815), the enzyme protein is retarded in the normal transport pathway (ER→Golgi apparatus→endosomes→lysosome) and prematurely degraded. Therefore, a compound which binds to and increases the stability of a mutant enzyme, may serve as a “chaperone” for the enzyme and increase the amount that can exit the ER and move to the lysosomes. In addition, because the folding and trafficking of some wild-type proteins is incomplete, with up to 70% of some wild-type proteins being degraded in some instances prior to reaching their final cellular location, the chaperones can be used to stabilize wild-type enzymes and increase the amount of enzyme which can exit the ER and be trafficked to lysosomes.

In one or more embodiments, the pharmacological chaperone comprises migalastat or a salt thereof. The compound migalastat, also known as 1-deoxygalactonojirimycin (1-DGJ) or (2R,3S,4R,55)-2-(hydroxymethyl) piperdine-3,4,5-triol is a compound having the following chemical formula:

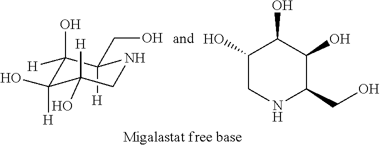 [SMILES: OCC1NCC(O)C(O)C1O ; OC[C@H]1NC[C@H](O)[C@@H](O)[C@H]1O]

As discussed herein, pharmaceutically acceptable salts of migalastat may also be used in the present invention. When a salt of migalastat is used, the dosage of the salt will be adjusted so that the dose of migalastat received by the patient is equivalent to the amount which would have been received had the migalastat free base been used. One example of a pharmaceutically acceptable salt of migalastat is migalastat HCl:

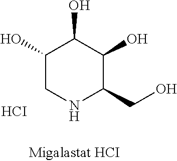 [SMILES: OC[C@H]1NC[C@H](O)[C@@H](O)[C@H]1O ; "HCI"]

Migalastat is a low molecular weight iminosugar and is an analogue of the terminal galactose of GL-3. In vitro and in vivo pharmacologic studies have demonstrated that migalastat acts as a pharmacological chaperone, selectively and reversibly binding, with high affinity, to the active site of wild-type (WT) α-Gal A and specific mutant forms of a Gal A, the genotypes of which are referred to as HEK assay amenable mutations. Migalastat binding stabilizes these mutant forms of α-Gal A in the endoplasmic reticulum facilitating their proper trafficking to lysosomes where dissociation of migalastat allows α-Gal A to reduce the level of GL-3 and other substrates. Approximately 30-50% of patients with Fabry disease have HEK assay amenable mutations; the majority of which are associated with the classic phenotype of the disease. A list of HEK assay amenable mutations includes at least those mutations listed in Table 1 below. In one or more embodiments, if a double mutation is present on the same chromosome (males and females), that patient is considered HEK assay amenable if the double mutation is present in one entry in Table 1 (e.g., D55V/Q57L). In some embodiments, if a double mutation is present on different chromosomes (only in females) that patient is considered HEK assay amenable if either one of the individual mutations is present in Table 1. In addition to Table 1 below, HEK assay amenable mutations can also be found in the summary of product characteristics and/or prescribing information for GALAFOLD™ in various countries in which GALAFOLD™ is approved for use, or at the website www.galafoldamenabilitytable.com, each of which is hereby incorporated by reference in its entirety.

TABLE 1
Amenable mutations
Table 1

| | | Protein |
|---|---|---|
| Nucleotide change | Nucleotide change | sequence change |
| c.7C > G | c.C7G | L3V |
| c.8T > C | c.T8C | L3P |
| c.[11G > T; 620A > C] | c.G11T/A620C | R4M/Y207S |
| c.13A > G | c.A13G | N5D |
| c.15C > G | c.C15G | N5K |
| c.16C > A | c.C16A | P6T |
| c.16C > T | c.C16T | P6S |
| c.17C > A | c.C17A | P6Q |
| c.17C > G | c.C17G | P6R |
| c.17C > T | c.C17T | P6L |
| c.19G > A | c.G19A | E7K |
| c.20A > T | c.A20T | E7V |
| c.21A > T | c.A21T | E7D |
| c.22C > A | c.C22A | L8I |
| c.23T > A | c.T23A | L8Q |
| c.23T > C | c.T23C | L8P |
| c.25C > T | c.C25T | H9Y |
| c.26A > G | c.A26G | H9R |
| c.26A > T | c.A26T | H9L |
| c.27T > A | c.T27A | H9Q |
| c.28C > A | c.C28A | L10M |
| c.28C > G | c.C28G | L10V |
| c.29T > A | c.T29A | L10Q |
| c.29T > C | c.T29C | L10P |
| c.29T > G | c.T29G | L10R |
| c.31G > A | c.G31A | G11S |
| c.31G > C | c.G31C | G11R |
| c.31G > T | c.G31T | G11C |
| c.32G > A | c.G32A | G11D |
| c.32G > T | c.G32T | G11V |
| c.34T > A | c.T34A | C12S |
| c.34T > C | c.T34C | C12R |
| c.34T > G | c.T34G | C12G |
| c.35G > A | c.G35A | C12Y |
| c.37G > A | c.G37A | A13T |
| c.37G > C | c.G37C | A13P |
| c.38C > A | c.C38A | A13E |
| c.38C > G | c.C38G | A13G |
| c.40C > G | c.C40G | L14V |
| c.40C > T | c.C40T | L14F |
| c.41T > A | c.T41A | L14H |
| c.43G > A | c.G43A | A15T |
| c.44C > G | c.C44G | A15G |
| c.49C > A | c.C49A | R17S |
| c.49C > G | c.C49G | R17G |
| c.49C > T | c.C49T | R17C |
| c.50G > A | c.G50A | R17H |
| c.50G > C | c.G50C | R17P |
| c.52T > A | c.T52A | F18I |
| c.53T > G | c.T53G | F18C |
| c.54C > G | c.C54G | F18L |
| c.58G > C | c.G58C | A20P |
| c.59C > A | c.C59A | A20D |
| c.59C > G | c.C59G | A20G |
| c.62T > A | c.T62A | L21H |
| c.64G > A | c.G64A | V22I |
| c.64G > C | c.G64C | V22L |
| c.64G > T | c.G64T | V22F |
| c.65T > C | c.T65C | V22A |
| c.65T > G | c.T65G | V22G |
| c.67T > A | c.T67A | S23T |
| c.67T > C | c.T67C | S23P |
| c.70T > C or c.70T > A | c.T70C or c.T70A | W24R |
| c.70T > G | c.T70G | W24G |
| c.71G > C | c.G71C | W24S |
| c.72G > C or c.72G > T | c.G72C or c.G72T | W24C |
| c.73G > C | c.G73C | D25H |
| c.77T > A | c.T77A | I26N |
| c.79C > A | c.C79A | P27T |
| c.79C > G | c.C79G | P27A |
| c.79C > T | c.C79T | P27S |
| c.80C > T | c.C80T | P27L |
| c.82G > C | c.G82C | G28R |
| c.82G > T | c.G82T | G28W |
| c.83G > A | c.G83A | G28E |
| c.85G > C | c.G85C | A29P |
| c.86C > A | c.C86A | A29D |
| c.86C > G | c.C86G | A29G |
| c.86C > T | c.C86T | A29V |
| c.88A > G | c.A88G | R30G |
| c.94C > A | c.C94A | L32M |
| c.94C > G | c.C94G | L32V |
| c.95T > A | c.T95A | L32Q |
| c.95T > C | c.T95C | L32P |
| c.95T > G | c.T95G | L32R |
| c.97G > C | c.G97C | D33H |
| c.97G > T | c.G97T | D33Y |
| c.98A > C | c.A98C | D33A |
| c.98A > G | c.A98G | D33G |
| c.98A > T | c.A98T | D33V |
| c.99C > G | c.C99G | D33E |
| c.100A > C | c.A100C | N34H |
| c.100A > G | c.A100G | N34D |
| c.101A > C | c.A101C | N34T |
| c.101A > G | c.A101G | N34S |
| c.102T > G or c.102T > A | c.T102G or c.T102A | N34K |
| c.103G > C or c.103G > A | c.G103C or c.G103A | G35R |
| c.104G > A | c.G104A | G35E |
| c.104G > C | c.G104C | G35A |
| c.104G > T | c.G104T | G35V |
| c.106T > A | c.T106A | L36M |
| c.106T > G | c.T106G | L36V |
| c.107T > C | c.T107C | L36S |
| c.107T > G | c.T107G | L36W |
| c.108G > C or c.108G > T | c.G108C or c.G108T | L36F |
| c.109G > A | c.G109A | A37T |
| c.109G > T | c.G109T | A37S |
| c.110C > A | c.C110A | A37E |
| c.110C > G | c.C110G | A37G |
| c.110C > T | c.C110T | A37V |
| c.112A > G | c.A112G | R38G |
| c.112A > T | c.A112T | R38W |
| c.113G > T | c.G113T | R38M |
| c.114G > C | c.G114C | R38S |
| c.115A > G | c.A115G | T39A |
| c.115A > T | c.A115T | T39S |
| c.116C > A | c.C116A | T39K |
| c.116C > G | c.C116G | T39R |
| c.116C > T | c.C116T | T39M |
| c.121A > G | c.A121G | T41A |
| c.122C > A | c.C122A | T41N |
| c.122C > G | c.C122G | T41S |
| c.122C > T | c.C122T | T41I |
| c.124A > C or c.124A > T | c.A124C or c.A124T | M42L |
| c.124A > G | c.A124G | M42V |
| c.125T > A | c.T125A | M42K |
| c.125T > C | c.T125C | M42T |
| c.125T > G | c.T125G | M42R |
| c.126G > A or c.126G > C or c.126G > T | c.G126A or c.G126C or c.G126T | M42I |
| c.128G > C | c.G128C | G43A |
| c.133C > A | c.C133A | L45M |
| c.133C > G | c.C133G | L45V |
| c.136C > A | c.C136A | H46N |
| c.136C > G | c.C136G | H46D |
| c.137A > C | c.A137C | H46P |
| c.138C > G | c.C138G | H46Q |
| c.142G > C | c.G142C | E48Q |
| c.143A > C | c.A143C | E48A |
| c.149T > A | c.T149A | F50Y |
| c.151A > G | c.A151G | M51V |
| c.152T > A | c.T152A | M51K |
| c.152T > C | c.T152C | M51T |
| c.152T > G | c.T152G | M51R |
| c.153G > A or c.153G > T or c.153G > C | c.G153A or c.G153T or c.G153C | M51I |
| c.157A > C | c.A157C | N53H |
| c.[157A > C; 158A > T] | c.A157C/A158T | N53L |
| c.157A > G | c.A157G | N53D |
| c.157A > T | c.A157T | N53Y |
| c.158A > C | c.A158C | N53T |
| c.158A > G | c.A158G | N53S |
| c.158A > T | c.A158T | N53I |
| c.159C > G or c.159C > A | c.C159G or c.C159A | N53K |
| c.160C > G | c.C160G | L54V |
| c.160C > T | c.C160T | L54F |
| c.161T > A | c.T161A | L54H |
| c.161T > C | c.T161C | L54P |
| c.161T > G | c.T161G | L54R |
| c.163G > C | c.G163C | D55H |
| c.163G > T | c.G163T | D55Y |
| c.164A > C | c.A164C | D55A |
| c.164A > G | c.A164G | D55G |
| c.164A > T | c.A164T | D55V |
| c.[164A > T; 170A > T] | c.A164T/A170T | D55V/Q57L |
| c.165C > G | c.C165G | D55E |
| c.167G > A | c.G167A | C56Y |
| c.167G > T | c.G167T | C56F |
| c.168C > G | c.C168G | C56W |
| c.170A > G | c.A170G | Q57R |
| c.170A > T | c.A170T | Q57L |
| c.172G > A | c.G172A | E58K |
| c.175G > A | c.G175A | E59K |
| c.175G > C | c.G175C | E59Q |
| c.176A > C | c.A176C | E59A |
| c.176A > G | c.A176G | E59G |
| c.176A > T | c.A176T | E59V |
| c.177G > C | c.G177C | E59D |
| c.178C > A | c.C178A | P60T |
| c.178C > G | c.C178G | P60A |
| c.178C > T | c.C178T | P60S |
| c.179C > A | c.C179A | P60Q |
| c.179C > G | c.C179G | P60R |
| c.179C > T | c.C179T | P60L |
| c.182A > T | c.A182T | D61V |
| c.183T > A | c.T183A | D61E |
| c.184_185insTAG | c.184_185insTAG | S62delinsLA |
| c.184T > C | c.T184C | S62P |
| c.184T > G | c.T184G | S62A |
| c.185C > A | c.C185A | S62Y |
| c.185C > G | c.C185G | S62C |
| c.185C > T | c.C185T | S62F |
| c.190A > C | c.A190C | I64L |
| c.190A > G | c.A190G | I64V |
| c.193A > G | c.A193G | S65G |
| c.193A > T | c.A193T | S65C |
| c.195T > A | c.T195A | S65R |
| c.196G > A | c.G196A | E66K |
| c.197A > G | c.A197G | E66G |
| c.197A > T | c.A197T | E66V |
| c.198G > C | c.G198C | E66D |
| c.199A > C | c.A199C | K67Q |
| c.199A > G | c.A199G | K67E |
| c.200A > C | c.A200C | K67T |
| c.200A > T | c.A200T | K67M |
| c.201G > C | c.G201C | K67N |
| c.202C > A | c.C202A | L68I |
| c.205T > A | c.T205A | F69I |
| c.206T > A | c.T206A | F69Y |
| c.207C > A or c.207C > G | c.C207A or c.C207G | F69L |
| c.208A > T | c.A208T | M70L |
| c.209T > A | c.T209A | M70K |
| c.209T > G | c.T209G | M70R |
| c.210G > C | c.G210C | M70I |
| c.211G > C | c.G211C | E71Q |
| c.212A > C | c.A212C | E71A |
| c.212A > G | c.A212G | E71G |
| c.212A > T | c.A212T | E71V |
| c.213G > C | c.G213C | E71D |
| c.214A > G | c.A214G | M72V |
| c.214A > T | c.A214T | M72L |
| c.215T > C | c.T215C | M72T |
| c.216G > A or c.216G > T or c.216G > C | c.G216A or c.G216T or c.G216C | M72I |
| c.217G > A | c.G217A | A73T |
| c.217G > T | c.G217T | A73S |
| c.218C > T | c.C218T | A73V |
| c.220G > A | c.G220A | E74K |
| c.221A > G | c.A221G | E74G |
| c.221A > T | c.A221T | E74V |
| c.222G > C | c.G222C | E74D |
| c.223C > T | c.C223T | L75F |
| c.224T > C | c.T224C | L75P |
| c.226A > G | c.A226G | M76V |
| c.227T > C | c.T227C | M76T |
| c.229G > A | c.G229A | V77I |
| c.229G > C | c.G229C | V77L |
| c.232T > C | c.T232C | S78P |
| c.233C > T | c.C233T | S78L |
| c.235G > A | c.G235A | E79K |
| c.235G > C | c.G235C | E79Q |
| c.236A > C | c.A236C | E79A |
| c.236A > G | c.A236G | E79G |
| c.236A > T | c.A236T | E79V |
| c.237A > T | c.A237T | E79D |
| c.238G > A | c.G238A | G80S |
| c.238G > T | c.G238T | G80C |
| c.239G > A | c.G239A | G80D |
| c.239G > C | c.G239C | G80A |
| c.239G > T | c.G239T | G80V |
| c.242G > T | c.G242T | W81L |
| c.244A > G | c.A244G | K82E |
| c.245A > C | c.A245C | K82T |
| c.245A > G | c.A245G | K82R |
| c.245A > T | c.A245T | K82M |
| c.246G > C | c.G246C | K82N |
| c.247G > A | c.G247A | D83N |
| c.248A > C | c.A248C | D83A |
| c.248A > G | c.A248G | D83G |
| c.248A > T | c.A248T | D83V |
| c.249T > A | c.T249A | D83E |
| c.250G > A | c.G250A | A84T |
| c.250G > C | c.G250C | A84P |
| c.250G > T | c.G250T | A84S |
| c.251C > A | c.C251A | A84E |
| c.251C > G | c.C251G | A84G |
| c.251C > T | c.C251T | A84V |
| c.253G > A | c.G253A | G85S |
| c.[253G > A; 254G > A] | c.G253A/G254A | G85N |
| c.[253G > A; 254G > T; 255T > G] | c.G253A/G254T/T255G | G85M |
| c.253G > C | c.G253C | G85R |
| c.253G > T | c.G253T | G85C |
| c.254G > A | c.G254A | G85D |
| c.254G > C | c.G254C | G85A |
| c.257A > T | c.A257T | Y86F |
| c.260A > G | c.A260G | E87G |
| c.261G > C or c.261G > T | c.G261C or c.G261T | E87D |
| c.262T > A | c.T262A | Y88N |
| c.262T > C | c.T262C | Y88H |
| c.263A > C | c.A263C | Y88S |
| c.263A > G | c.A263G | Y88C |
| c.265C > G | c.C265G | L89V |
| c.265C > T | c.C265T | L89F |
| c.271A > C | c.A271C | I91L |
| c.271A > T | c.A271T | I91F |
| c.272T > C | c.T272C | I91T |
| c.272T > G | c.T272G | I91S |
| c.273T > G | c.T273G | I91M |
| c.286A > G | c.A286G | M96V |
| c.286A > T | c.A286T | M96L |
| c.287T > C | c.T287C | M96T |
| c.288G > A or c.288G > T or c.288G > C | c.G288A or c.G288T or c.G288C | M96I |
| c.289G > A | c.G289A | A97T |
| c.289G > C | c.G289C | A97P |
| c.289G > T | c.G289T | A97S |
| c.290C > A | c.C290A | A97D |
| c.290C > T | c.C290T | A97V |
| c.293C > A | c.C293A | P98H |
| c.293C > G | c.C293G | P98R |
| c.293C > T | c.C293T | P98L |
| c.295C > G | c.C295G | Q99E |
| c.296A > C | c.A296C | Q99P |
| c.296A > G | c.A296G | Q99R |
| c.296A > T | c.A296T | Q99L |
| c.301G > C | c.G301C | D101H |
| c.302A > C | c.A302C | D101A |
| c.302A > G | c.A302G | D101G |
| c.302A > T | c.A302T | D101V |
| c.303T > A | c.T303A | D101E |
| c.304T > A | c.T304A | S102T |
| c.304T > C | c.T304C | S102P |
| c.304T > G | c.T304G | S102A |
| c.305C > T | c.C305T | S102L |
| c.310G > A | c.G310A | G104S |
| c.311G > A | c.G311A | G104D |
| c.311G > C | c.G311C | G104A |
| c.311G > T | c.G311T | G104V |
| c.313A > G | c.A313G | R105G |
| c.314G > A | c.G314A | R105K |
| c.314G > C | c.G314C | R105T |
| c.314G > T | c.G314T | R105I |
| c.316C > A | c.C316A | L106I |
| c.316C > G | c.C316G | L106V |
| c.316C > T | c.C316T | L106F |
| c.317T > A | c.T317A | L106H |
| c.317T > C | c.T317C | L106P |
| c.319C > A | c.C319A | Q107K |
| c.319C > G | c.C319G | Q107E |
| c.320A > G | c.A320G | Q107R |
| c.321G > C | c.G321C | Q107H |
| c.322G > A | c.G322A | A108T |
| c.323C > A | c.C323A | A108E |
| c.323C > T | c.C323T | A108V |
| c.325G > A | c.G325A | D109N |
| c.325G > C | c.G325C | D109H |
| c.325G > T | c.G325T | D109Y |
| c.326A > C | c.A326C | D109A |
| c.326A > G | c.A326G | D109G |
| c.327C > G | c.C327G | D109E |
| c.328C > A | c.C328A | P110T |
| c.334C > G | c.C334G | R112G |
| c.335G > A | c.G335A | R112H |
| c.335G > T | c.G335T | R112L |
| c.337T > A | c.T337A | F113I |
| c.337T > C or c.339T > A or c.339T > G | c.T337C or c.T339A or c.T339G | F113L |
| c.337T > G | c.T337G | F113V |
| c.338T > A | c.T338A | F113Y |
| c.341C > T | c.C341T | P114L |
| c.343C > A | c.C343A | H115N |
| c.343C > G | c.C343G | H115D |
| c.346G > C | c.G346C | G116R |
| c.350T > C | c.T350C | I117T |
| c.351T > G | c.T351G | I117M |
| c.352C > T | c.C352T | R118C |
| c.361G > A | c.G361A | A121T |
| c.362C > T | c.C362T | A121V |
| c.367T > A | c.T367A | Y123N |
| c.367T > G | c.T367G | Y123D |
| c.368A > C | c.A368C | Y123S |
| c.368A > G | c.A368G | Y123C |
| c.368A > T | c.A368T | Y123F |
| c.370G > A | c.G370A | V124I |
| c.371T > G | c.T371G | V124G |
| c.373C > A | c.C373A | H125N |
| c.373C > G | c.C373G | H125D |
| c.373C > T | c.C373T | H125Y |
| c.374A > G | c.A374G | H125R |
| c.374A > T | c.A374T | H125L |
| c.376A > G | c.A376G | S126G |
| c.376A > T | c.A376T | S126C |
| c.377G > T | c.G377T | S126I |
| c.379A > G | c.A379G | K127E |
| c.383G > A | c.G383A | G128E |
| c.383G > C | c.G383C | G128A |
| c.385C > G | c.C385G | L129V |
| c.388A > C | c.A388C | K130Q |
| c.389A > T | c.A389T | K130M |
| c.390G > C | c.G390C | K130N |
| c.391C > G | c.C391G | L131V |
| c.397A > C | c.A397C | I133L |
| c.397A > G | c.A397G | I133V |
| c.397A > T | c.A397T | I133F |
| c.398T > C | c.T398C | I133T |
| c.399T > G | c.T399G | I133M |
| c.[399T > G; 434T > C] | c.T399G/T434C | I133M/F145S |
| c.403G > A | c.G403A | A135T |
| c.403G > T | c.G403T | A135S |
| c.404C > A | c.C404A | A135E |
| c.404C > G | c.C404G | A135G |
| c.404C > T | c.C404T | A135V |
| c.406G > A | c.G406A | D136N |
| c.407A > C | c.A407C | D136A |
| c.407A > T | c.A407T | D136V |
| c.408T > A or c.408T > G | c.T408A or c.T408G | D136E |
| c.409G > A | c.G409A | V137I |
| c.409G > C | c.G409C | V137L |
| c.410T > A | c.T410A | V137D |
| c.410T > C | c.T410C | V137A |
| c.410T > G | c.T410G | V137G |
| c.413G > C | c.G413C | G138A |
| c.415A > C | c.A415C | N139H |
| c.415A > T | c.A415T | N139Y |
| c.416A > G | c.A416G | N139S |
| c.416A > T | c.A416T | N139I |
| c.417T > A | c.T417A | N139K |
| c.418A > C | c.A418C | K140Q |
| c.418A > G | c.A418G | K140E |
| c.419A > C | c.A419C | K140T |
| c.419A > G | c.A419G | K140R |
| c.419A > T | c.A419T | K140I |
| c.420A > T | c.A420T | K140N |
| c.421A > T | c.A421T | T141S |
| c.427G > A | c.G427A | A143T |
| c.428C > A | c.C428A | A143E |
| c.428C > G | c.C428G | A143G |
| c.428C > T | c.C428T | A143V |
| c.430G > A | c.G430A | G144S |
| c.430G > C | c.G430C | G144R |
| c.430G > T | c.G430T | G144C |
| c.431G > A | c.G431A | G144D |
| c.431G > C | c.G431C | G144A |
| c.431G > T | c.G431T | G144V |
| c.433T > G | c.T433G | F145V |
| c.434T > A | c.T434A | F145Y |
| c.434T > C | c.T434C | F145S |
| c.434T > G | c.T434G | F145C |
| c.435C > G | c.C435G | F145L |
| c.436C > A | c.C436A | P146T |
| c.436C > G | c.C436G | P146A |
| c.436C > T | c.C436T | P146S |
| c.437C > A | c.C437A | P146H |
| c.437C > G | c.C437G | P146R |
| c.437C > T | c.C437T | P146L |
| c.440G > C | c.G440C | G147A |
| c.442A > G | c.A442G | S148G |
| c.442A > T | c.A442T | S148C |
| c.443G > C | c.G443C | S148T |
| c.446T > G | c.T446G | F149C |
| c.449G > A | c.G449A | G150E |
| c.449G > T | c.G449T | G150V |
| c.451T > G | c.T451G | Y151D |
| c.452A > C | c.A452C | Y151S |
| c.452A > G | c.A452G | Y151C |
| c.454T > A | c.T454A | Y152N |
| c.454T > C | c.T454C | Y152H |
| c.454T > G | c.T454G | Y152D |
| c.455A > C | c.A455C | Y152S |
| c.455A > G | c.A455G | Y152C |
| c.455A > T | c.A455T | Y152F |
| c.457G > A | c.G457A | D153N |
| c.457G > C | c.G457C | D153H |
| c.457G > T | c.G457T | D153Y |
| c.458A > C | c.A458C | D153A |
| c.458A > T | c.A458T | D153V |
| c.465T > A or c.465T > G | c.T465A or c.T465G | D155E |
| c.466G > A | c.G466A | A156T |
| c.466G > T | c.G466T | A156S |
| c.467C > G | c.C467G | A156G |
| c.467C > T | c.C467T | A156V |
| c.469C > A | c.C469A | Q157K |
| c.469C > G | c.C469G | Q157E |
| c.470A > C | c.A470C | Q157P |
| c.470A > T | c.A470T | Q157L |
| c.471G > C or c.471G > T | c.G471C or c.G471T | Q157H |
| c.472A > G | c.A472G | T158A |
| c.472A > T | c.A472T | T158S |
| c.473C > A | c.C473A | T158N |
| c.473C > T | c.C473T | T158I |
| c.475T > A | c.T475A | F159I |
| c.475T > G | c.T475G | F159V |
| c.476T > A | c.T476A | F159Y |
| c.476T > G | c.T476G | F159C |
| c.477T > A | c.T477A | F159L |
| c.478G > A | c.G478A | A160T |
| c.478G > T | c.G478T | A160S |
| c.479C > A | c.C479A | A160D |
| c.479C > G | c.C479G | A160G |
| c.479C > T | c.C479T | A160V |
| c.481G > A | c.G481A | D161N |
| c.481G > C | c.G481C | D161H |
| c.481G > T | c.G481T | D161Y |
| c.482A > T | c.A482T | D161V |
| c.484T > G | c.T484G | W162G |
| c.485G > C | c.G485C | W162S |
| c.490G > A | c.G490A | V164I |
| c.490G > T | c.G490T | V164L |
| c.491T > C | c.T491C | V164A |
| c.493G > A | c.G493A | D165N |
| c.493G > C | c.G493C | D165H |
| c.494A > C | c.A494C | D165A |
| c.494A > G | c.A494G | D165G |
| c.495T > A | c.T495A | D165E |
| c.496_497delinsTC | c.496_497delinsTC | L166S |
| c.496C > A | c.C496A | L166M |
| c.496C > G | c.C496G | L166V |
| c.[496C > G; 497T > G] | c.C496G/T497G | L166G |
| c.497T > A | c.T497A | L166Q |
| c.499C > A | c.C499A | L167I |
| c.499C > G | c.C499G | L167V |
| c.505T > A | c.T505A | F169I |
| c.505T > G | c.T505G | F169V |
| c.506T > A | c.T506A | F169Y |
| c.506T > C | c.T506C | F169S |
| c.506T > G | c.T506G | F169C |
| c.507T > A | c.T507A | F169L |
| c.511G > A | c.G511A | G171S |
| c.512G > C | c.G512C | G171A |
| c.512G > T | c.G512T | G171V |
| c.517T > C | c.T517C | Y173H |
| c.518A > C | c.A518C | Y173S |
| c.518A > G | c.A518G | Y173C |
| c.518A > T | c.A518T | Y173F |
| c.520T > C | c.T520C | C174R |
| c.520T > G | c.T520G | C174G |
| c.523G > C | c.G523C | D175H |
| c.523G > T | c.G523T | D175Y |
| c.524A > G | c.A524G | D175G |
| c.524A > T | c.A524T | D175V |
| c.525C > G or c.525C > A | c.C525G or c.C525A | D175E |
| c.526A > T | c.A526T | S176C |
| c.528T > A | c.T528A | S176R |
| c.529T > A | c.T529A | L177M |
| c.529T > G | c.T529G | L177V |
| c.530T > C | c.T530C | L177S |
| c.530T > G | c.T530G | L177W |
| c.531G > C | c.G531C | L177F |
| c.532G > A | c.G532A | E178K |
| c.532G > C | c.G532C | E178Q |
| c.533A > C | c.A533C | E178A |
| c.533A > G | c.A533G | E178G |
| c.538T > A | c.T538A | L180M |
| c.538T > G | c.T538G | L180V |
| c.539T > C | c.T539C | L180S |
| c.539T > G | c.T539G | L180W |
| c.540G > C or c.540G > T | c.G540C or c.G540T | L180F |
| c.541G > A | c.G541A | A181T |
| c.541G > C | c.G541C | A181P |
| c.542C > T | c.C542T | A181V |
| c.544G > T | c.G544T | D182Y |
| c.545A > C | c.A545C | D182A |
| c.545A > G | c.A545G | D182G |
| c.545A > T | c.A545T | D182V |
| c.546T > A | c.T546A | D182E |
| c.548G > A | c.G548A | G183D |
| c.548G > C | c.G548C | G183A |
| c.550T > A | c.T550A | Y184N |
| c.550T > C | c.T550C | Y184H |
| c.551A > C | c.A551C | Y184S |
| c.551A > G | c.A551G | Y184C |
| c.551A > T | c.A551T | Y184F |
| c.553A > C | c.A553C | K185Q |
| c.553A > G | c.A553G | K185E |
| c.554A > C | c.A554C | K185T |
| c.554A > T | c.A554T | K185M |
| c.555G > C | c.G555C | K185N |
| c.556C > A | c.C556A | H186N |
| c.556C > G | c.C556G | H186D |
| c.556C > T | c.C556T | H186Y |
| c.557A > T | c.A557T | H186L |
| c.558C > G | c.C558G | H186Q |
| c.559_564dup | c.559_564dup | p.M187_S188dup |
| c.559A > T | c.A559T | M187L |
| c.559A > G | c.A559G | M187V |
| c.560T > C | c.T560C | M187T |
| c.561G > T or c.561G > A or c.561G > C | c.G561T or c.G561A or c.G561C | M187I |
| c.562T > A | c.T562A | S188T |
| c.562T > C | c.T562C | S188P |
| c.562T > G | c.T562G | S188A |
| c.563C > A | c.C563A | S188Y |
| c.563C > G | c.C563G | S188C |
| c.563C > T | c.C563T | S188F |
| c.565T > G | c.T565G | L189V |
| c.566T > C | c.T566C | L189S |
| c.567G > C or c.567G > T | c.G567C or c.G567T | L189F |
| c.568G > A | c.G568A | A190T |
| c.568G > T | c.G568T | A190S |
| c.569C > A | c.C569A | A190D |
| c.569C > G | c.C569G | A190G |
| c.569C > T | c.C569T | A190V |
| c.571C > A | c.C571A | L191M |
| c.571C > G | c.C571G | L191V |
| c.572T > A | c.T572A | L191Q |
| c.574A > C | c.A574C | N192H |
| c.574A > G | c.A574G | N192D |
| c.575A > C | c.A575C | N192T |
| c.575A > G | c.A575G | N192S |
| c.576T > A | c.T576A | N192K |
| c.577A > G | c.A577G | R193G |
| c.577A > T | c.A577T | R193W |
| c.578G > C | c.G578C | R193T |
| c.578G > T | c.G578T | R193M |
| c.580A > C | c.A580C | T194P |
| c.580A > G | c.A580G | T194A |
| c.580A > T or c.581C > G | c.A580T or c.C581G | T194S |
| c.581C > A | c.C581A | T194N |
| c.581C > T | c.C581T | T194I |
| c.583G > A | c.G583A | G195S |
| c.583G > C | c.G583C | G195R |
| c.583G > T | c.G583T | G195C |
| c.584G > T | c.G584T | G195V |
| c.586A > G | c.A586G | R196G |
| c.587G > A | c.G587A | R196K |
| c.587G > C | c.G587C | R196T |
| c.587G > T | c.G587T | R196I |
| c.589A > G | c.A589G | S197G |
| c.589A > T | c.A589T | S197C |
| c.590G > A | c.G590A | S197N |
| c.590G > C | c.G590C | S197T |
| c.590G > T | c.G590T | S197I |
| c.593T > C | c.T593C | I198T |
| c.593T > G | c.T593G | I198S |
| c.594T > G | c.T594G | I198M |
| c.595G > A | c.G595A | V199M |
| c.595G > C | c.G595C | V199L |
| c.596T > A | c.T596A | V199E |
| c.596T > C | c.T596C | V199A |
| c.596T > G | c.T596G | V199G |
| c.598T > A | c.T598A | Y200N |
| c.599A > C | c.A599C | Y200S |
| c.599A > G | c.A599G | Y200C |
| c.601T > A | c.T601A | S201T |
| c.601T > G | c.T601G | S201A |
| c.602C > A | c.C602A | S201Y |
| c.602C > G | c.C602G | S201C |
| c.602C > T | c.C602T | S201F |
| c.607G > C | c.G607C | E203Q |
| c.608A > C | c.A608C | E203A |
| c.608A > G | c.A608G | E203G |
| c.608A > T | c.A608T | E203V |
| c.609G > C or c.609G > T | c.G609C or c.G609T | E203D |
| c.610T > G | c.T610G | W204G |
| c.611G > C | c.G611C | W204S |
| c.611G > T | c.G611T | W204L |
| c.613C > A | c.C613A | P205T |
| c.613C > T | c.C613T | P205S |
| c.614C > T | c.C614T | P205L |
| c.616C > A | c.C616A | L206I |
| c.616C > G | c.C616G | L206V |
| c.616C > T | c.C616T | L206F |
| c.617T > A | c.T617A | L206H |
| c.617T > G | c.T617G | L206R |
| c.619T > C | c.T619C | Y207H |
| c.620A > C | c.A620C | Y207S |
| c.620A > T | c.A620T | Y207F |
| c.623T > A | c.T623A | M208K |
| c.623T > G | c.T623G | M208R |
| c.625T > A | c.T625A | W209R |
| c.625T > G | c.T625G | W209G |
| c.627G > C | c.G627C | W209C |
| c.628C > A | c.C628A | P210T |
| c.628C > T | c.C628T | P210S |
| c.629C > A | c.C629A | P210H |
| c.629C > T | c.C629T | P210L |
| c.631T > C | c.T631C | F211L |
| c.631T > G | c.T631G | F211V |
| c.632T > A | c.T632A | F211Y |
| c.632T > C | c.T632C | F211S |
| c.632T > G | c.T632G | F211C |
| c.635A > C | c.A635C | Q212P |
| c.636A > T | c.A636T | Q212H |
| c.637A > C | c.A637C | K213Q |
| c.637A > G | c.A637G | K213E |
| c.638A > G | c.A638G | K213R |
| c.638A > T | c.A638T | K213M |
| c.640C > A | c.C640A | P214T |
| c.640C > G | c.C640G | P214A |
| c.640C > T | c.C640T | P214S |
| c.641C > A | c.C641A | P214H |
| c.641C > G | c.C641G | P214R |
| c.641C > T | c.C641T | P214L |
| c.643A > C | c.A643C | N215H |
| c.643A > G | c.A643G | N215D |
| c.643A > T | c.A643T | N215Y |
| c.644A > C | c.A644C | N215T |
| c.644A > G | c.A644G | N215S |
| c.[644A > G; 937G > T] | c.A644G/G937T | N215S/D313Y |
| c.644A > T | c.A644T | N215I |
| c.645T > A | c.T645A | N215K |
| c.646T > A | c.T646A | Y216N |
| c.646T > C | c.T646C | Y216H |
| c.646T > G | c.T646G | Y216D |
| c.647A > C | c.A647C | Y216S |
| c.647A > G | c.A647G | Y216C |
| c.647A > T | c.A647T | Y216F |
| c.649A > C | c.A649C | T217P |
| c.649A > G | c.A649G | T217A |
| c.649A > T | c.A649T | T217S |
| c.650C > A | c.C650A | T217K |
| c.650C > G | c.C650G | T217R |
| c.650C > T | c.C650T | T217I |
| c.652G > A | c.G652A | E218K |
| c.652G > C | c.G652C | E218Q |
| c.653A > C | c.A653C | E218A |
| c.653A > G | c.A653G | E218G |
| c.653A > T | c.A653T | E218V |
| c.654A > T | c.A654T | E218D |
| c.655A > C | c.A655C | I219L |
| c.655A > T | c.A655T | I219F |
| c.656T > A | c.T656A | I219N |
| c.656T > C | c.T656C | I219T |
| c.656T > G | c.T656G | I219S |
| c.657C > G | c.C657G | I219M |
| c.659G > A | c.G659A | R220Q |
| c.659G > C | c.G659C | R220P |
| c.659G > T | c.G659T | R220L |
| c.661C > A | c.C661A | Q221K |
| c.661C > G | c.C661G | Q221E |
| c.662A > C | c.A662C | Q221P |
| c.662A > G | c.A662G | Q221R |
| c.662A > T | c.A662T | Q221L |
| c.663G > C | c.G663C | Q221H |
| c.664T > A | c.T664A | Y222N |
| c.664T > C | c.T664C | Y222H |
| c.664T > G | c.T664G | Y222D |
| c.665A > C | c.A665C | Y222S |
| c.665A > G | c.A665G | Y222C |
| c.670A > C | c.A670C | N224H |
| c.671A > C | c.A671C | N224T |
| c.671A > G | c.A671G | N224S |
| c.673C > G | c.C673G | H225D |
| c.679C > G | c.C679G | R227G |
| c.682A > C | c.A682C | N228H |
| c.682A > G | c.A682G | N228D |
| c.683A > C | c.A683C | N228T |
| c.683A > G | c.A683G | N228S |
| c.683A > T | c.A683T | N228I |
| c.685T > A | c.T685A | F229I |
| c.686T > A | c.T686A | F229Y |
| c.686T > C | c.T686C | F229S |
| c.687T > A or c.687T > G | c.T687A or c.T687G | F229L |
| c.688G > C | c.G688C | A230P |
| c.689C > A | c.C689A | A230D |
| c.689C > G | c.C689G | A230G |
| c.689C > T | c.C689T | A230V |
| c.694A > C | c.A694C | I232L |
| c.694A > G | c.A694G | I232V |
| c.695T > C | c.T695C | I232T |
| c.696T > G | c.T696G | I232M |
| c.698A > C | c.A698C | D233A |
| c.698A > G | c.A698G | D233G |
| c.698A > T | c.A698T | D233V |
| c.699T > A | c.T699A | D233E |
| c.703T > A | c.T703A | S235T |
| c.703T > G | c.T703G | S235A |
| c.710A > T | c.A710T | K237I |
| c.712A > G | c.A712G | S238G |
| c.712A > T | c.A712T | S238C |
| c.713G > A | c.G713A | S238N |
| c.713G > C | c.G713C | S238T |
| c.713G > T | c.G713T | S238I |
| c.715A > T | c.A715T | I239L |
| c.716T > C | c.T716C | I239T |
| c.717A > G | c.A717G | I239M |
| c.718A > G | c.A718G | K240E |
| c.719A > G | c.A719G | K240R |
| c.719A > T | c.A719T | K240M |
| c.720G > C or c.720G > T | c.G720C or c.G720T | K240N |
| c.721A > T | c.A721T | S241C |
| c.722G > C | c.G722C | S241T |
| c.722G > T | c.G722T | S241I |
| c.724A > C | c.A724C | I242L |
| c.724A > G | c.A724G | I242V |
| c.724A > T | c.A724T | I242F |
| c.725T > A | c.T725A | I242N |
| c.725T > C | c.T725C | I242T |
| c.725T > G | c.T725G | I242S |
| c.726C > G | c.C726G | I242M |
| c.727T > A | c.T727A | L243M |
| c.727T > G | c.T727G | L243V |
| c.728T > C | c.T728C | L243S |
| c.728T > G | c.T728G | L243W |
| c.729G > C or c.729G > T | c.G729C or c.G729T | L243F |
| c.730G > A | c.G730A | D244N |
| c.730G > C | c.G730C | D244H |
| c.730G > T | c.G730T | D244Y |
| c.731A > C | c.A731C | D244A |
| c.731A > G | c.A731G | D244G |
| c.731A > T | c.A731T | D244V |
| c.732C > G | c.C732G | D244E |
| c.733T > G | c.T733G | W245G |
| c.735G > C | c.G735C | W245C |
| c.736A > G | c.A736G | T246A |
| c.737C > A | c.C737A | T246K |
| c.737C > G | c.C737G | T246R |
| c.737C > T | c.C737T | T246I |
| c.739T > A | c.T739A | S247T |
| c.739T > G | c.T739G | S247A |
| c.740C > A | c.C740A | S247Y |
| c.740C > G | c.C740G | S247C |
| c.740C > T | c.C740T | S247F |
| c.742T > G | c.T742G | F248V |
| c.743T > A | c.T743A | F248Y |
| c.743T > G | c.T743G | F248C |
| c.744T > A | c.T744A | F248L |
| c.745A > C | c.A745C | N249H |
| c.745A > G | c.A745G | N249D |
| c.745A > T | c.A745T | N249Y |
| c.746A > C | c.A746C | N249T |
| c.746A > G | c.A746G | N249S |
| c.746A > T | c.A746T | N249I |
| c.747C > G or c.747C > A | c.C747G or c.C747A | N249K |
| c.748C > A | c.C748A | Q250K |
| c.748C > G | c.C748G | Q250E |
| c.749A > C | c.A749C | Q250P |
| c.749A > G | c.A749G | Q250R |
| c.749A > T | c.A749T | Q250L |
| c.750G > C | c.G750C | Q250H |
| c.751G > A | c.G751A | E251K |
| c.751G > C | c.G751C | E251Q |
| c.752A > G | c.A752G | E251G |
| c.752A > T | c.A752T | E251V |
| c.754A > G | c.A754G | R252G |
| c.757A > G | c.A757G | I253V |
| c.757A > T | c.A757T | I253F |
| c.758T > A | c.T758A | I253N |
| c.758T > C | c.T758C | I253T |
| c.758T > G | c.T758G | I253S |
| c.760-762delGTT or c.761-763del | c.760_762delGTT or c.761_763del | p.V254del |
| c.760G > T | c.G760T | V254F |
| c.761T > A | c.T761A | V254D |
| c.761T > C | c.T761C | V254A |
| c.761T > G | c.T761G | V254G |
| c.763G > A | c.G763A | D255N |
| c.763G > C | c.G763C | D255H |
| c.763G > T | c.G763T | D255Y |
| c.764A > C | c.A764C | D255A |
| c.764A > T | c.A764T | D255V |
| c.765T > A | c.T765A | D255E |
| c.766G > C | c.G766C | V256L |
| c.767T > A | c.T767A | V256D |
| c.767T > G | c.T767G | V256G |
| c.769G > A | c.G769A | A257T |
| c.769G > C | c.G769C | A257P |
| c.769G > T | c.G769T | A257S |
| c.770C > G | c.C770G | A257G |
| c.770C > T | c.C770T | A257V |
| c.772G > C or c.772G > A | c.G772C or c.G772A | G258R |
| c.773G > A | c.G773A | G258E |
| c.773G > T | c.G773T | G258V |
| c.775C > A | c.C775A | P259T |
| c.775C > G | c.C775G | P259A |
| c.775C > T | c.C775T | P259S |
| c.776C > A | c.C776A | P259Q |
| c.776C > G | c.C776G | P259R |
| c.776C > T | c.C776T | P259L |
| c.778G > T | c.G778T | G260W |
| c.779G > A | c.G779A | G260E |
| c.779G > C | c.G779C | G260A |
| c.781G > A | c.G781A | G261S |
| c.781G > C | c.G781C | G261R |
| c.781G > T | c.G781T | G261C |
| c.782G > C | c.G782C | G261A |
| c.787A > C | c.A787C | N263H |
| c.788A > C | c.A788C | N263T |
| c.788A > G | c.A788G | N263S |
| c.790G > A | c.G790A | D264N |
| c.790G > C | c.G790C | D264H |
| c.790G > T | c.G790T | D264Y |
| c.793C > G | c.C793G | P265A |
| c.794C > A | c.C794A | P265Q |
| c.794C > T | c.C794T | P265L |
| c.799A > G | c.A799G | M267V |
| c.799A > T | c.A799T | M267L |
| c.800T > C | c.T800C | M267T |
| c.802T > A | c.T802A | L268I |
| c.804A > T | c.A804T | L268F |
| c.805G > A | c.G805A | V269M |
| c.805G > C | c.G805C | V269L |
| c.806T > C | c.T806C | V269A |
| c.808A > C | c.A808C | I270L |
| c.808A > G | c.A808G | I270V |
| c.809T > C | c.T809C | I270T |
| c.809T > G | c.T809G | I270S |
| c.810T > G | c.T810G | I270M |
| c.811G > A | c.G811A | G271S |
| c.[811G > A; 937G > T] | c.G811A/G937T | G271S/D313Y |
| c.812G > A | c.G812A | G271D |
| c.812G > C | c.G812C | G271A |
| c.814A > G | c.A814G | N272D |
| c.818T > A | c.T818A | F273Y |
| c.823C > A | c.C823A | L275I |
| c.823C > G | c.C823G | L275V |
| c.827G > A | c.G827A | S276N |
| c.827G > C | c.G827C | S276T |
| c.829T > G | c.T829G | W277G |
| c.830G > T | c.G830T | W277L |
| c.831G > T or c.831G > C | c.G831T or c.G831C | W277C |
| c.832A > T | c.A832T | N278Y |
| c.833A > T | c.A833T | N278I |
| c.835C > G | c.C835G | Q279E |
| c.838C > A | c.C838A | Q280K |
| c.839A > G | c.A839G | Q280R |
| c.839A > T | c.A839T | Q280L |
| c.840A > T or c.840A > C | c.A840T or c.A840C | Q280H |
| c.841G > C | c.G841C | V281L |
| c.842T > A | c.T842A | V281E |
| c.842T > C | c.T842C | V281A |
| c.842T > G | c.T842G | V281G |
| c.844A > G | c.A844G | T282A |
| c.844A > T | c.A844T | T282S |
| c.845C > T | c.C845T | T282I |
| c.847C > G | c.C847G | Q283E |
| c.848A > T | c.A848T | Q283L |
| c.849G > C | c.G849C | Q283H |
| c.850A > G | c.A850G | M284V |
| c.850A > T | c.A850T | M284L |
| c.851T > C | c.T851C | M284T |
| c.852G > C | c.G852C | M284I |
| c.853G > A | c.G853A | A285T |
| c.854C > G | c.C854G | A285G |
| c.854C > T | c.C854T | A285V |
| c.856C > G | c.C856G | L286V |
| c.856C > T | c.C856T | L286F |
| c.857T > A | c.T857A | L286H |
| c.860G > T | c.G860T | W287L |
| c.862G > C | c.G862C | A288P |
| c.862G > T | c.G862T | A288S |
| c.863C > G | c.C863G | A288G |
| c.863C > T | c.C863T | A288V |
| c.865A > C | c.A865C | I289L |
| c.865A > G | c.A865G | I289V |
| c.866T > C | c.T866C | I289T |
| c.866T > G | c.T866G | I289S |
| c.868A > C or c.868A > T | c.A868C or c.A868T | M290L |
| c.868A > G | c.A868G | M290V |
| c.869T > C | c.T869C | M290T |
| c.870G > A or c.870G > C or c.870G > T | c.G870A or c.G870C or c.G870T | M290I |
| c.871G > A | c.G871A | A291T |
| c.871G > T | c.G871T | A291S |
| c.872C > G | c.C872G | A291G |
| c.874G > T | c.G874T | A292S |
| c.875C > G | c.C875G | A292G |
| c.877C > A | c.C877A | P293T |
| c.880T > A | c.T880A | L294I |
| c.880T > G | c.T880G | L294V |
| c.881T > C | c.T881C | L294S |
| c.882A > T | c.A882T | L294F |
| c.883T > A | c.T883A | F295I |
| c.883T > G | c.T883G | F295V |
| c.884T > A | c.T884A | F295Y |
| c.884T > C | c.T884C | F295S |
| c.884T > G | c.T884G | F295C |
| c.886A > G | c.A886G | M296V |
| c.886A > T or c.886A > C | c.A886T or c.A886C | M296L |
| c.887T > C | c.T887C | M296T |
| c.888G > A or c.888G > T or c.888G > C | c.G888A or c.G888T or c.G888C | M296I |
| c.889T > A | c.T889A | S297T |
| c.892A > G | c.A892G | N298D |
| c.893A > C | c.A893C | N298T |
| c.893A > G | c.A893G | N298S |
| c.893A > T | c.A893T | N298I |
| c.895G > A | c.G895A | D299N |
| c.895G > C | c.G895C | D299H |
| c.897C > G or c.897C > A | c.C897G or c.C897A | D299E |
| c.898C > A | c.C898A | L300I |
| c.898C > G | c.C898G | L300V |
| c.898C > T | c.C898T | L300F |
| c.899T > C | c.T899C | L300P |
| c.901C > G | c.C901G | R301G |
| c.902G > A | c.G902A | R301Q |
| c.902G > C | c.G902C | R301P |
| c.902G > T | c.G902T | R301L |
| c.904C > A | c.C904A | H302N |
| c.904C > G | c.C904G | H302D |
| c.904C > T | c.C904T | H302Y |
| c.905A > T | c.A905T | H302L |
| c.907A > G | c.A907G | I303V |
| c.907A > T | c.A907T | I303F |
| c.908T > A | c.T908A | I303N |
| c.908T > C | c.T908C | I303T |
| c.908T > G | c.T908G | I303S |
| c.911G > A | c.G911A | S304N |
| c.911G > C | c.G911C | S304T |
| c.911G > T | c.G911T | S304I |
| c.916C > G | c.C916G | Q306E |
| c.917A > C | c.A917C | Q306P |
| c.917A > T | c.A917T | Q306L |
| c.919G > A | c.G919A | A307T |
| c.919G > C | c.G919C | A307P |
| c.919G > T | c.G919T | A307S |
| c.920C > A | c.C920A | A307D |
| c.920C > G | c.C920G | A307G |
| c.920C > T | c.C920T | A307V |
| c.922A > C | c.A922C | K308Q |
| c.922A > G | c.A922G | K308E |
| c.923A > G | c.A923G | K308R |
| c.923A > T | c.A923T | K308I |
| c.924A > T or c.924A > C | c.A924T or c.A924C | K308N |
| c.925G > A | c.G925A | A309T |
| c.925G > C | c.G925C | A309P |
| c.926C > A | c.C926A | A309D |
| c.926C > T | c.C926T | A309V |
| c.928C > A | c.C928A | L310I |
| c.928C > G | c.C928G | L310V |
| c.928C > T | c.C928T | L310F |
| c.931C > A | c.C931A | L311I |
| c.931C > G | c.C931G | L311V |
| c.934C > A | c.C934A | Q312K |
| c.934C > G | c.C934G | Q312E |
| c.935A > G | c.A935G | Q312R |
| c.935A > T | c.A935T | Q312L |
| c.936G > T or c.936G > C | c.G936T or c.G936C | Q312H |
| c.937G > T | c.G937T | D313Y |
| c.[937G > T; 1232G > A] | c.G937T/G1232A | D313Y/G411D |
| c.938A > G | c.A938G | D313G |
| c.938A > T | c.A938T | D313V |
| c.939T > A | c.T939A | D313E |
| c.940A > G | c.A940G | K314E |
| c.941A > C | c.A941C | K314T |
| c.941A > T | c.A941T | K314M |
| c.942G > C | c.G942C | K314N |
| c.943G > A | c.G943A | D315N |
| c.943G > C | c.G943C | D315H |
| c.943G > T | c.G943T | D315Y |
| c.944A > C | c.A944C | D315A |
| c.944A > G | c.A944G | D315G |
| c.944A > T | c.A944T | D315V |
| c.946G > A | c.G946A | V316I |
| c.946G > C | c.G946C | V316L |
| c.947T > C | c.T947C | V316A |
| c.947T > G | c.T947G | V316G |
| c.949A > C | c.A949C | I317L |
| c.949A > G | c.A949G | I317V |
| c.950T > C | c.T950C | I317T |
| c.951T > G | c.T951G | I317M |
| c.952G > A | c.G952A | A318T |
| c.952G > C | c.G952C | A318P |
| c.953C > A | c.C953A | A318D |
| c.953C > T | c.C953T | A318V |
| c.955A > T | c.A955T | I319F |
| c.956T > C | c.T956C | I319T |
| c.957C > G | c.C957G | I319M |
| c.958A > C | c.A958C | N320H |
| c.959A > C | c.A959C | N320T |
| c.959A > G | c.A959G | N320S |
| c.959A > T | c.A959T | N320I |
| c.961C > A | c.C961A | Q321K |
| c.962A > G | c.A962G | Q321R |
| c.962A > T | c.A962T | Q321L |
| c.963G > C or c.963G > T | c.G963C or c.G963T | Q321H |
| c.964G > A | c.G964A | D322N |
| c.964G > C | c.G964C | D322H |
| c.965A > C | c.A965C | D322A |
| c.965A > T | c.A965T | D322V |
| c.966C > A or c.966C > G | c.C966A or c.C966G | D322E |
| c.967C > A | c.C967A | P323T |
| c.968C > G | c.C968G | P323R |
| c.970T > G | c.T970G | L324V |
| c.971T > G | c.T971G | L324W |
| c.973G > A | c.G973A | G325S |
| c.973G > C | c.G973C | G325R |
| c.973G > T | c.G973T | G325C |
| c.974G > C | c.G974C | G325A |
| c.974G > T | c.G974T | G325V |
| c.976A > C | c.A976C | K326Q |
| c.976A > G | c.A976G | K326E |
| c.977A > C | c.A977C | K326T |
| c.977A > G | c.A977G | K326R |
| c.977A > T | c.A977T | K326M |
| c.978G > C or c.978G > T | c.G978C or c.G978T | K326N |
| c.979C > G | c.C979G | Q327E |
| c.980A > C | c.A980C | Q327P |
| c.980A > T | c.A980T | Q327L |
| c.981A > T | c.A981T | Q327H |
| c.983G > C | c.G983C | G328A |
| c.985T > A | c.T985A | Y329N |
| c.985T > C | c.T985C | Y329H |
| c.985T > G | c.T985G | Y329D |
| c.986A > G | c.A986G | Y329C |
| c.986A > T | c.A986T | Y329F |
| c.988C > A | c.C988A | Q330K |
| c.988C > G | c.C988G | Q330E |
| c.989A > C | c.A989C | Q330P |
| c.989A > G | c.A989G | Q330R |
| c.990G > C | c.G990C | Q330H |
| c.991C > G | c.C991G | L331V |
| c.992T > A | c.T992A | L331H |
| c.992T > C | c.T992C | L331P |
| c.992T > G | c.T992G | L331R |
| c.994A > G | c.A994G | R332G |
| c.995G > C | c.G995C | R332T |
| c.995G > T | c.G995T | R332I |
| c.996A > T | c.A996T | R332S |
| c.997C > G | c.C997G | Q333E |
| c.998A > C | c.A998C | Q333P |
| c.998A > T | c.A998T | Q333L |
| c.1000G > C | c.G1000C | G334R |
| c.1001G > A | c.G1001A | G334E |
| c.1001G > T | c.G1001T | G334V |
| c.1003G > T | c.G1003T | D335Y |
| c.1004A > C | c.A1004C | D335A |
| c.1004A > G | c.A1004G | D335G |
| c.1004A > T | c.A1004T | D335V |
| c.1005C > G | c.C1005G | D335E |
| c.1006A > G | c.A1006G | N336D |
| c.1006A > T | c.A1006T | N336Y |
| c.1007A > C | c.A1007C | N336T |
| c.1007A > G | c.A1007G | N336S |
| c.1007A > T | c.A1007T | N336I |
| c.1009T > G | c.T1009G | F337V |
| c.1010T > A | c.T1010A | F337Y |
| c.1010T > C | c.T1010C | F337S |
| c.1010T > G | c.T1010G | F337C |
| c.1011T > A | c.T1011A | F337L |
| c.1012G > A | c.G1012A | E338K |
| c.1013A > C | c.A1013C | E338A |
| c.1013A > G | c.A1013G | E338G |
| c.1013A > T | c.A1013T | E338V |
| c.1014A > T | c.A1014T | E338D |
| c.1015G > A | c.G1015A | V339M |
| c.1016T > A | c.T1016A | V339E |
| c.1016T > C | c.T1016C | V339A |
| c.1021G > C | c.G1021C | E341Q |
| c.1022A > C | c.A1022C | E341A |
| c.1027C > A | c.C1027A | P343T |
| c.1027C > G | c.C1027G | P343A |
| c.1027C > T | c.C1027T | P343S |
| c.1028C > T | c.C1028T | P343L |
| c.1030C > G | c.C1030G | L344V |
| c.1030C > T | c.C1030T | L344F |
| c.1031T > G | c.T1031G | L344R |
| c.1033T > C | c.T1033C | S345P |
| c.1036G > T | c.G1036T | G346C |
| c.1037G > A | c.G1037A | G346D |
| c.1037G > C | c.G1037C | G346A |
| c.1037G > T | c.G1037T | G346V |
| c.1039T > A | c.T1039A | L347I |
| c.1043C > A | c.C1043A | A348D |
| c.1046G > C | c.G1046C | W349S |
| c.1046G > T | c.G1046T | W349L |
| c.1047G > C | c.G1047C | W349C |
| c.1048G > A | c.G1048A | A350T |
| c.1048G > T | c.G1048T | A350S |
| c.1049C > G | c.C1049G | A350G |
| c.1049C > T | c.C1049T | A350V |
| c.1052T > A | c.T1052A | V351E |
| c.1052T > C | c.T1052C | V351A |
| c.1054G > A | c.G1054A | A352T |
| c.1054G > T | c.G1054T | A352S |
| c.1055C > G | c.C1055G | A352G |
| c.1055C > T | c.C1055T | A352V |
| c.1057A > T | c.A1057T | M353L |
| c.1058T > A | c.T1058A | M353K |
| c.1058T > C | c.T1058C | M353T |
| c.1061T > A | c.T1061A | I354K |
| c.1061T > G | c.T1061G | I354R |
| c.1063A > C | c.A1063C | N355H |
| c.1063A > G | c.A1063G | N355D |
| c.1063A > T | c.A1063T | N355Y |
| c.1064A > G | c.A1064G | N355S |
| c.1066C > G | c.C1066G | R356G |
| c.1066C > T | c.C1066T | R356W |
| c.1067G > A | c.G1067A | R356Q |
| c.1067G > C | c.G1067C | R356P |
| c.1067G > T | c.G1067T | R356L |
| c.1069C > G | c.C1069G | Q357E |
| c.1072G > C | c.G1072C | E358Q |
| c.1073A > C | c.A1073C | E358A |
| c.1073A > G | c.A1073G | E358G |
| c.1074G > T or c.1074G > C | c.G1074T or c.G1074C | E358D |
| c.1075A > C | c.A1075C | I359L |
| c.1075A > G | c.A1075G | I359V |
| c.1075A > T | c.A1075T | I359F |
| c.1076T > A | c.T1076A | I359N |
| c.1076T > C | c.T1076C | I359T |
| c.1076T > G | c.T1076G | I359S |
| c.1078G > A | c.G1078A | G360S |
| c.1078G > C | c.G1078C | G360R |
| c.1078G > T | c.G1078T | G360C |
| c.1079G > A | c.G1079A | G360D |
| c.1079G > C | c.G1079C | G360A |
| c.1082G > A | c.G1082A | G361E |
| c.1082G > C | c.G1082C | G361A |
| c.1084C > A | c.C1084A | P362T |
| c.1084C > G | c.C1084G | P362A |
| c.1084C > T | c.C1084T | P362S |
| c.1085C > A | c.C1085A | P362H |
| c.1085C > G | c.C1085G | P362R |
| c.1085C > T | c.C1085T | P362L |
| c.1087C > A | c.C1087A | R363S |
| c.1087C > G | c.C1087G | R363G |
| c.1087C > T | c.C1087T | R363C |
| c.1088G > A | c.G1088A | R363H |
| c.1088G > T | c.G1088T | R363L |
| c.1090T > C | c.T1090C | S364P |
| c.1091C > G | c.C1091G | S364C |
| c.1093T > A | c.T1093A | Y365N |
| c.1093T > G | c.T1093G | Y365D |
| c.1094A > C | c.A1094C | Y365S |
| c.1094A > T | c.A1094T | Y365F |
| c.1096A > C | c.A1096C | T366P |
| c.1096A > T | c.A1096T | T366S |
| c.1097C > A | c.C1097A | T366N |
| c.1097C > T | c.C1097T | T366I |
| c.1099A > C | c.A1099C | I367L |
| c.1099A > T | c.A1099T | I367F |
| c.1101C > G | c.C1101G | I367M |
| c.1102G > A | c.G1102A | A368T |
| c.1102G > C | c.G1102C | A368P |
| c.1103C > G | c.C1103G | A368G |
| c.1105G > A | c.G1105A | V369I |
| c.1105G > C | c.G1105C | V369L |
| c.1105G > T | c.G1105T | V369F |
| c.1106T > C | c.T1106C | V369A |
| c.1106T > G | c.T1106G | V369G |
| c.1108G > A | c.G1108A | A370T |
| c.1108G > C | c.G1108C | A370P |
| c.1109C > A | c.C1109A | A370D |
| c.1109C > G | c.C1109G | A370G |
| c.1109C > T | c.C1109T | A370V |
| c.1111T > A | c.T1111A | S371T |
| c.1112C > G | c.C1112G | S371C |
| c.1117G > A | c.G1117A | G373S |
| c.1117G > T | c.G1117T | G373C |
| c.1118G > C | c.G1118C | G373A |
| c.1120A > G | c.A1120G | K374E |
| c.1121A > C | c.A1121C | K374T |
| c.1121A > G | c.A1121G | K374R |
| c.1121A > T | c.A1121T | K374I |
| c.1123G > C | c.G1123C | G375R |
| c.1124G > A | c.G1124A | G375E |
| c.1124G > C | c.G1124C | G375A |
| c.1126G > A | c.G1126A | V376M |
| c.1126G > C | c.G1126C | V376L |
| c.1127T > A | c.T1127A | V376E |
| c.1127T > G | c.T1127G | V376G |
| c.1129G > A | c.G1129A | A377T |
| c.1129G > C | c.G1129C | A377P |
| c.1129G > T | c.G1129T | A377S |
| c.1130C > G | c.C1130G | A377G |
| c.1135A > G | c.A1135G | N379D |
| c.1136A > C | c.A1136C | N379T |
| c.1136A > T | c.A1136T | N379I |
| c.1137T > A | c.T1137A | N379K |
| c.1138C > A | c.C1138A | P380T |
| c.1138C > G | c.C1138G | P380A |
| c.1139C > A | c.C1139A | P380H |
| c.1139C > G | c.C1139G | P380R |
| c.1139C > T | c.C1139T | P380L |
| c.1142C > A | c.C1142A | A381D |
| c.1147T > A | c.T1147A | F383I |
| c.1148T > A | c.T1148A | F383Y |
| c.1148T > G | c.T1148G | F383C |
| c.1150A > T | c.A1150T | I384F |
| c.1151T > C | c.T1151C | I384T |
| c.1152C > G | c.C1152G | I384M |
| c.1153A > G | c.A1153G | T385A |
| c.1154C > T | c.C1154T | T385I |
| c.1156C > A | c.C1156A | Q386K |
| c.1157A > T | c.A1157T | Q386L |
| c.1158G > C | c.G1158C | Q386H |
| c.1159C > A | c.C1159A | L387I |
| c.1159C > T | c.C1159T | L387F |
| c.1160T > A | c.T1160A | L387H |
| c.1160T > G | c.T1160G | L387R |
| c.1162C > A | c.C1162A | L388I |
| c.1162C > G | c.C1162G | L388V |
| c.1162C > T | c.C1162T | L388F |
| c.1163T > A | c.T1163A | L388H |
| c.1163T > G | c.T1163G | L388R |
| c.1168G > A | c.G1168A | V390M |
| c.1171A > C | c.A1171C | K391Q |
| c.1171A > G | c.A1171G | K391E |
| c.1172A > C | c.A1172C | K391T |
| c.1172A > G | c.A1172G | K391R |
| c.1172A > T | c.A1172T | K391I |
| c.1173A > T | c.A1173T | K391N |
| c.1174A > G | c.A1174G | R392G |
| c.1174A > T | c.A1174T | R392W |
| c.1175G > A | c.G1175A | R392K |
| c.1175G > C | c.G1175C | R392T |
| c.1175G > T | c.G1175T | R392M |
| c.1177A > C | c.A1177C | K393Q |
| c.1177A > G | c.A1177G | K393E |
| c.1178A > C | c.A1178C | K393T |
| c.1179G > C | c.G1179C | K393N |
| c.1180C > A | c.C1180A | L394I |
| c.1181T > A | c.T1181A | L394Q |
| c.1181T > C | c.T1181C | L394P |
| c.1181T > G | c.T1181G | L394R |
| c.1183G > C | c.G1183C | G395R |
| c.1184G > A | c.G1184A | G395E |
| c.1184G > C | c.G1184C | G395A |
| c.1186T > A | c.T1186A | F396I |
| c.1186T > G | c.T1186G | F396V |
| c.1187T > G | c.T1187G | F396C |
| c.1188C > G | c.C1188G | F396L |
| c.1189T > A | c.T1189A | Y397N |
| c.1189T > C | c.T1189C | Y397H |
| c.1190A > C | c.A1190C | Y397S |
| c.1190A > G | c.A1190G | Y397C |
| c.1190A > T | c.A1190T | Y397F |
| c.1192G > A | c.G1192A | E398K |
| c.1192G > C | c.G1192C | E398Q |
| c.1193A > G | c.A1193G | E398G |
| c.1195T > A | c.T1195A | W399R |
| c.1195T > G | c.T1195G | W399G |
| c.1198A > C | c.A1198C | T400P |
| c.1198A > G | c.A1198G | T400A |
| c.1198A > T | c.A1198T | T400S |
| c.1199C > A | c.C1199A | T400N |
| c.1199C > T | c.C1199T | T400I |
| c.1201T > A | c.T1201A | S401T |
| c.1201T > G | c.T1201G | S401A |
| c.1202_1203insGACTTC | c.1202_1203insGACTTC | p.T400_S401dup |
| c.1202C > T | c.C1202T | S401L |
| c.1204A > G | c.A1204G | R402G |
| c.1204A > T | c.A1204T | R402W |
| c.1205G > C | c.G1205C | R402T |
| c.1205G > T | c.G1205T | R402M |
| c.1206G > C | c.G1206C | R402S |
| c.1207T > G | c.T1207G | L403V |
| c.1208T > C | c.T1208C | L403S |
| c.1209A > T | c.A1209T | L403F |
| c.1210A > G | c.A1210G | R404G |
| c.1211G > A | c.G1211A | R404K |
| c.1211G > C | c.G1211C | R404T |
| c.1211G > T | c.G1211T | R404I |
| c.1212A > T | c.A1212T | R404S |
| c.1213A > G | c.A1213G | S405G |
| c.1216C > G | c.C1216G | H406D |
| c.1217A > T | c.A1217T | H406L |
| c.1218C > G | c.C1218G | H406Q |
| c.1219A > T | c.A1219T | I407L |
| c.1220T > C | c.T1220C | I407T |
| c.1221A > G | c.A1221G | I407M |
| c.1222A > C | c.A1222C | N408H |
| c.1222A > G | c.A1222G | N408D |
| c.1222A > T | c.A1222T | N408Y |
| c.1223A > C | c.A1223C | N408T |
| c.1225C > A | c.C1225A | P409T |
| c.1225C > G | c.C1225G | P409A |
| c.1225C > T | c.C1225T | P409S |
| c.1226C > T | c.C1226T | P409L |
| c.1228A > G | c.A1228G | T410A |
| c.1228A > T | c.A1228T | T410S |
| c.1229C > T | c.C1229T | T410I |
| c.1231G > A | c.G1231A | G411S |
| c.1231G > T | c.G1231T | G411C |
| c.1232G > A | c.G1232A | G411D |
| c.1232G > C | c.G1232C | G411A |
| c.1232G > T | c.G1232T | G411V |
| c.1234A > C | c.A1234C | T412P |
| c.1234A > G | c.A1234G | T412A |
| c.1234A > T | c.A1234T | T412S |
| c.1235C > A | c.C1235A | T412N |
| c.1235C > T | c.C1235T | T412I |
| c.1237G > A | c.G1237A | V413I |
| c.1237G > T | c.G1237T | V413F |
| c.1238T > G | c.T1238G | V413G |
| c.1240T > G | c.T1240G | L414V |
| c.1242G > C | c.G1242C | L414F |
| c.1243C > A | c.C1243A | L415I |
| c.1244T > A | c.T1244A | L415H |
| c.1246C > G | c.C1246G | Q416E |
| c.1247A > T | c.A1247T | Q416L |
| c.1248G > C | c.G1248C | Q416H |
| c.1249C > A | c.C1249A | L417I |
| c.1252G > A | c.G1252A | E418K |
| c.1252G > C | c.G1252C | E418Q |
| c.1253A > C | c.A1253C | E418A |
| c.1253A > G | c.A1253G | E418G |
| c.1254A > T | c.A1254T | E418D |
| c.1255A > G | c.A1255G | N419D |
| c.1255A > T | c.A1255T | N419Y |
| c.1256A > C | c.A1256C | N419T |
| c.1256A > G | c.A1256G | N419S |
| c.1256A > T | c.A1256T | N419I |
| c.1258A > C | c.A1258C | T420P |
| c.1258A > T | c.A1258T | T420S |
| c.1259C > A | c.C1259A | T420K |
| c.1259C > G | c.C1259G | T420R |
| c.1261A > G | c.A1261G | M421V |
| c.1261A > T | c.A1261T | M421L |
| c.1262T > A | c.T1262A | M421K |
| c.1262T > C | c.T1262C | M421T |
| c.1262T > G | c.T1262G | M421R |
| c.1263G > C | c.G1263C | M421I |
| c.1265A > C | c.A1265C | Q422P |
| c.1267A > T | c.A1267T | M423L |
| c.1268T > A | c.T1268A | M423K |
| c.1268T > C | c.T1268C | M423T |
| c.1269G > C | c.G1269C | M423I |
| c.1271C > T | c.C1271T | S424L |
| c.1275A > C | c.A1275C | L425F |
| c.1279G > A | c.G1279A | D427N |
| c.1286T > G | c.T1286G | L429R |

Kidney Function in Fabry Patients

Progressive decline in renal function is a major complication of Fabry disease. For example, patients associated with a classic Fabry phenotype exhibit progressive renal impairment that can lead to dialysis or renal transplantation.

A frequently used method in the art to assess kidney function is the glomerular filtration rate (GFR). Generally, the GFR is the volume of fluid filtered from the renal glomerular capillaries into the Bowman's capsule per unit time. Clinically, estimates of GFR are made based upon the clearance of creatinine from serum. GFR can be estimated by collecting urine to determine the amount of creatinine that was removed from the blood over a given time interval. Age, body size and gender may also be factored in. The lower the GFR number, the more advanced kidney damage is.

Some studies indicate that untreated Fabry patients experience an average decline in GFR between 7.0 and 18.9 mL/min/1.73 m² per year, while patients receiving an enzyme replacement therapy (ERT) may experience an average decline in GFR between 2.0 and 2.7 mL/min/1.73 m² per year, although more rapid declines may occur in patients with more significant proteinuria or with more severe chronic kidney disease. Thus, even with patients receiving therapy there is a need to determine an appropriate dose of the therapeutic to account for a patient's developing impairment of renal function. Adjustment of the dose can be used to avoid an accumulation of the therapeutic to a level that is outside the therapeutic index or to a level where the patient experiences toxicity.

An estimated GFR (eGFR) is calculated from serum creatinine using an isotope dilution mass spectrometry (IDMS) traceable equation. Two of the most commonly used equations for estimating glomerular filtration rate (GFR) from serum creatinine are the Chronic Kidney Disease Epidemiology Collaboration (CKD-EPI) equation and the Modification of Diet in Renal Disease (MDRD) Study equation. Both the MDRD Study and CKD-EPI equations include variables for age, gender, and race, which may allow providers to observe that CKD is present despite a serum creatinine concentration that appears to fall within or just above the normal reference interval.

The CKD-EPI equation uses a 2-slope “spline” to model the relationship between GFR and serum creatinine, age, sex, and race. CKD-EPI equation expressed as a single equation:

GFR=141×min (S_{cr}/κ,1)α×max(S_{cr}/κ1)−1.209×0.993×^{Age}×1.018[if female]×1.159[if black]

where:

S_{cr} is serum creatinine in mg/dL,

κ is 0.7 for females and 0.9 for males,

α is −0.329 for females and −0.411 for males,

min indicates the minimum of S_{cr}/κ or 1, and

max indicates the maximum of S_{cr}/κ or 1.

The following is the IDMS-traceable MDRD Study equation (for creatinine methods calibrated to an IDMS reference method):

GFR (mL/min/1.73 m²)=175×(S_{cr})^{-1.154}×(Age)^{-0.203}×(0.742 if female)×(1.212 if African American)

The equation does not require weight or height variables because the results are reported normalized to 1.73 m² body surface area, which is an accepted average adult surface area. The equation has been validated extensively in Caucasian and African American populations between the ages of 18 and 70 with impaired kidney function (eGFR <60 mL/min/1.73 m²) and has shown good performance for patients with all common causes of kidney disease.

One method for estimating the creatinine clearance rate (eC_{Cr}) is using the Cockcroft-Gault equation, which in turn estimates GFR in ml/min:

Creatinine Clearance (ml/min)=[(140−Age)×Mass(kg)*]÷72×Serum Creatinine (mg/dL)[*multiplied by 0.85 if female]

The Cockcroft-Gault equation is the equation suggested for use by the Food and Drug Administration for renal impairment studies. It is common for the creatinine clearance calculated by the Cockcroft-Gault formula to be normalized for a body surface area of 1.73 m². Therefore, this equation can be expressed as the estimated eGFR in mL/min/1.73 m². The normal range of GFR, adjusted for body surface area, is 100-130 ml/min/1.73 m² in men and 90-120 ml/min/1.73 m² in women younger than the age of 40.

The severity of chronic kidney disease has been defined in six stages (see also Table 2): (Stage 0) Normal kidney function—GFR above 90 mL/min/1.73 m² and no proteinuria; (Stage 1)—GFR above 90 mL/min/1.73 m² with evidence of kidney damage; (Stage 2) (mild)—GFR of 60 to 89 mL/min/1.73 m² with evidence of kidney damage; (Stage 3) (moderate)—GFR of 30 to 59 mL/min/1.73 m²; (Stage 4) (severe)—GFR of 15 to 29 mL/min/1.73 m²; (Stage 5) kidney failure−GFR less than 15 mL/min/1.73 m². Table 2 below shows the various kidney disease stages with corresponding GFR levels.

TABLE 2

| Chronic Kidney Disease | GFR level |
|---|---|
| Stage | (mL/min/1.73 m²) |
| Stage 1 (Normal) | ≥90 |
| Stage 2 (Mild) | 60-89 |
| Stage 3 (Moderate) | 30-59 |
| Stage 4 (Severe) | 15-29 |
| Stage 5 (Kidney Failure) | <15 |

Dosing, Formulation and Administration

One or more of the dosing regimens described herein are particularly suitable for Fabry patients who have some degree of renal impairment. Amicus Therapeutics has sponsored two Phase 3 studies using migalastat 150 mg every other day (QOD) in Fabry patients. FACETS (011, NCT00925301) was a 24-month trial, including a 6-month double-blind, placebo-controlled period, in 67 enzyme replacement therapy (ERT)-naive patients. ATTRACT (012, NCT01218659) was an active-controlled, 18-month trial in 57 ERT-experienced patients with a 12-month open-label extension (OLE). Both the FACETS and ATTRACT studies included patients having an estimated glomerular filtration rate (eGFR) of ≥30 ml/min/1.73 m². Accordingly, both studies included Fabry patients with normal renal function as well as patients with mild and moderate renal impairment, but neither study included patients with severe renal impairment.

The Phase 3 studies of migalastat treatment of Fabry patients established that 150 mg every other day slowed the progression of the disease as shown by surrogate markers. However, in some embodiments, the migalastat dosing regimen may be adjusted in some Fabry patients because these patients can experience kidney deterioration. With a slowing in the ability to clear the drug from the body there can be an increasing exposure to the patient to the drug. Thus, in some embodiments a dose adjustment protocol is provided to inform physicians of the best dose taking into consideration the current clearance profile from the body. Dose adjustment is particularly difficult with a chaperone because it is an inhibitor, and a delicate balance must be reached such that the chaperone is present in amounts great enough to be therapeutic, but also not so great that the chaperone inhibits enzyme function (which would exacerbate the disease). As such, it is difficult to predict correct dosing, which is further complicated in patients who have reduced capacity to clear the migalastat.

Accordingly, in one or more embodiments, the Fabry patient with renal impairment is administered about 100 mg to about 300 mg FBE of migalastat or salt thereof at a frequency of once every other day, once every three days, once every four days, once every five days, once every six days or once every seven days. In one or more embodiments, the migalastat or salt thereof is administered at a frequency of once every other day (also referred to as “QOD” or “Q48H”), every four days (also referred to as “Q4D” or “Q96H”) or every seven days (also referred to as “Q7D” or “Q168H”). In some embodiments, the Fabry patient with renal impairment is administered about 100 mg to about 300 mg FBE of migalastat or salt thereof at a frequency of once every four days. In other embodiments, the Fabry patient with renal impairment is administered about 100 mg to about 300 mg FBE of migalastat or salt thereof at a frequency of once every seven days. In some embodiments, dosing regimens of longer intervals (e.g. every three days to every seven days) may be begun after, or as an adjustment to, a dosing regimen of about 100 mg to about 300 mg FBE of migalastat or salt thereof at a frequency of once every other day.

In various embodiments, the doses described herein pertain to migalastat hydrochloride or an equivalent dose of migalastat or a salt thereof other than the hydrochloride salt. In some embodiments, these doses pertain to the free base of migalastat. In alternate embodiments, these doses pertain to a salt of migalastat. In further embodiments, the salt of migalastat is migalastat hydrochloride. The administration of migalastat or a salt of migalastat is referred to herein as “migalastat therapy”.

The effective amount of migalastat or salt thereof can be in the range from about 100 mg FBE to about 300 mg FBE. Exemplary doses include about 100 mg FBE, about 105 mg FBE, about 110 mg FBE, about 115 mg FBE, about 120 mg FBE, about 123 mg FBE, about 125 mg FBE, about 130 mg FBE, about 135 mg FBE, about 140 mg FBE, about 145 mg FBE, about 150 mg FBE, about 155 mg FBE, about 160 mg FBE, about 165 mg FBE, about 170 mg FBE, about 175 mg FBE, about 180 mg FBE, about 185 mg FBE, about 190 mg FBE, about 195 mg FBE, about 200 mg FBE, about 205 mg FBE, about 210 mg FBE, about 215 mg FBE, about 220 mg FBE, about 225 mg FBE, about 230 mg FBE, about 235 mg FBE, about 240 mg FBE, about 245 mg FBE, about 250 mg FBE, about 255 mg FBE, about 260 mg FBE, about 265 mg FBE, about 270 mg FBE, about 275 mg FBE, about 280 mg FBE, about 285 mg FBE, about 290 mg FBE, about 295 mg FBE or about 300 mg FBE.

Again, it is noted that 150 mg of migalastat hydrochloride is equivalent to 123 mg of the free base form of migalastat. Thus, in one or more embodiments, the dose is 150 mg of migalastat hydrochloride or an equivalent dose of migalastat or a salt thereof other than the hydrochloride salt, administered at a frequency of once every other day. As set forth above, this dose is referred to as 123 mg FBE of migalastat. In further embodiments, the dose is 150 mg of migalastat hydrochloride administered at a frequency of once every other day. In other embodiments, the dose is 123 mg of the migalastat free base administered at a frequency of once every other day.

In various embodiments, the effective amount is about 122 mg, about 128 mg, about 134 mg, about 140 mg, about 146 mg, about 150 mg, about 152 mg, about 159 mg, about 165 mg, about 171 mg, about 177 mg, about 183 mg, about 189 mg, about 195 mg, about 201 mg, about 207 mg, about 213 mg, about 220 mg, about 226 mg, about 232 mg, about 238 mg, about 244 mg, about 250 mg, about 256 mg, about 262 mg, about 268 mg, about 274 mg, about 280 mg, about 287 mg, about 293 mg, about 299 mg, about 305 mg, about 311 mg, about 317 mg, about 323 mg, about 329 mg, about 335 mg, about 341 mg, about 348 mg, about 354 mg, about 360 mg or about 366 mg of migalastat hydrochloride.

Thus, in one or more embodiments, the dose is 150 mg migalastat hydrochloride or an equivalent dose of migalastat or a salt thereof other than the hydrochloride salt, administered at a frequency of once every four days or once every seven days. In further embodiments, the dose is 150 mg migalastat hydrochloride administered every four days. In other embodiments, the dose is 150 mg 1 migalastat hydrochloride administered every seven days. In other embodiments, the dose is 123 mg of migalastat free base administered at a frequency of once every other day, once every four days or once every seven days. Longer dosing intervals (e.g. every three to seven days) may be useful with a higher degree of renal impairment compared to a dosing frequency of every other day. Such longer dosing intervals include every three, four, five, six or seven days.

In some embodiments, dosing intervals may include any dosing interval with more than 48 hours between doses. For example, dosing intervals may include dosing every 72, 96, 120, 144, or 168 hours.

In some embodiments, dosing intervals may include administration less than 3.5 times per week on average. For example, dosing may occur 3 times per week, 2 times per week or once per week on average. In some embodiments, dosing may occur, on average, less than or equal to about 2.3 times per week, less than or equal to about 1.75 times per week, less than or equal to about 1.4 times per week, or less than or equal to about 1.167 times per week.

In some embodiments, dosing intervals may be irregular. For example, dosing intervals may include administration every Monday, Wednesday and Friday, without administration on Tuesday, Thursday, Saturday or Sunday. Similarly, dosing intervals may include administration every Monday and Thursday, without administration on other days.

The administration of migalastat may be for a certain period of time. In one or more embodiments, the migalastat is administered for a duration of at least 28 days, such as at least 30, 60 or 90 days or at least 3, 4, 5, 6, 7, 8, 9, 10, 11, 12, 16, 20 or 24 months or at least 1, 2 or 3 years. In various embodiments, the migalastat therapy is long-term migalastat therapy of at least 6 months, such as at least 6, 7, 8, 9, 10, 11, 12, 16, 20 or 24 months or at least 1, 2 or 3 years.

Administration of migalastat according to the present invention may be in a formulation suitable for any route of administration, but is preferably administered in an oral dosage form such as a tablet, capsule or solution. As one example, the patient is orally administered capsules each containing 25 mg, 50 mg, 75 mg, 100 mg or 150 mg migalastat hydrochloride (i.e. 1-deoxygalactonojirimycin hydrochloride) or an equivalent dose of migalastat or a salt thereof other than the hydrochloride salt.

In some embodiments, the PC (e.g., migalastat or salt thereof) is administered orally. In one or more embodiments, the PC (e.g., migalastat or salt thereof) is administered by injection. The PC may be accompanied by a pharmaceutically acceptable carrier, which may depend on the method of administration.

In one embodiment of the invention, the chaperone compound is administered as monotherapy, and can be in a form suitable for any route of administration, including e.g., orally in the form tablets or capsules or liquid, or in sterile aqueous solution for injection. In other embodiments, the PC is provided in a dry lyophilized powder to be added to the formulation of the replacement enzyme during or immediately after reconstitution to prevent enzyme aggregation in vitro prior to administration.

When the chaperone compound is formulated for oral administration, the tablets or capsules can be prepared by conventional means with pharmaceutically acceptable excipients such as binding agents (e.g. pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active chaperone compound.

The pharmaceutical formulations of the chaperone compound suitable for parenteral/injectable use generally include sterile aqueous solutions (where water soluble), or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating the purified enzyme and the chaperone compound in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The formulation can contain an excipient. Pharmaceutically acceptable excipients which may be included in the formulation are buffers such as citrate buffer, phosphate buffer, acetate buffer, bicarbonate buffer, amino acids, urea, alcohols, ascorbic acid, and phospholipids; proteins, such as serum albumin, collagen, and gelatin; salts such as EDTA or EGTA, and sodium chloride; liposomes; polyvinylpyrollidone; sugars, such as dextran, mannitol, sorbitol, and glycerol; propylene glycol and polyethylene glycol (e.g., PEG-4000, PEG-6000); glycerol; glycine or other amino acids; and lipids. Buffer systems for use with the formulations include citrate; acetate; bicarbonate; and phosphate buffers. Phosphate buffer is a preferred embodiment.

The route of administration of the chaperone compound may be oral (preferably) or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation.

Administration of the above-described parenteral formulations of the chaperone compound may be by periodic injections of a bolus of the preparation, or may be administered by intravenous or intraperitoneal administration from a reservoir which is external (e.g., an i.v. bag) or internal (e.g., a bioerodable implant).

Embodiments relating to pharmaceutical formulations and administration may be combined with any of the other embodiments of the invention, for example embodiments relating to a method of treating a patient with Fabry disease, a method of enhancing α-galactosidase A in a patient diagnosed with or suspected of having Fabry disease, use of a pharmacological chaperone for α-galactosidase A for the manufacture of a medicament for treating a patient diagnosed with Fabry disease or to a pharmacological chaperone for α-galactosidase A for use in treating a patient diagnosed with Fabry disease as well as embodiments relating to amenable mutations, the PCs and suitable dosages thereof.

In one or more embodiments, chaperone is administered in combination with enzyme replacement therapy. Enzyme replacement therapy increases the amount of protein by exogenously introducing wild-type or biologically functional enzyme by way of infusion. This therapy has been developed for many genetic disorders, including lysosomal storage disorders such as Fabry disease, as referenced above. After the infusion, the exogenous enzyme is expected to be taken up by tissues through non-specific or receptor-specific mechanism. In general, the uptake efficiency is not high, and the circulation time of the exogenous protein is short. In addition, the exogenous protein is unstable and subject to rapid intracellular degradation as well as having the potential for adverse immunological reactions with subsequent treatments. In one or more embodiments, the chaperone is administered at the same time as replacement enzyme. In some embodiments, the chaperone is co-formulated with the replacement enzyme.

In one or more embodiments, a patient is switched from enzyme replace therapy (ERT) to migalastat therapy. In some embodiments, a patient on ERT is identified, the patient's ERT is discontinued, and the patient begins receiving migalastat therapy. The migalastat therapy can be in accordance with any of the methods described herein. In various embodiments, the patient has some degree of renal impairment, such as mild, moderate or severe renal impairment.

Monitoring Lyso-Gb3 and Migalastat Levels

Lyso-Gb3 (globotriaosylsphingosine) can be monitored to determine whether substrate is being cleared from the body of a Fabry patient. Higher levels of lyso-Gb3 correlate with higher levels of substrate. If a patient is being successfully treated, then lyso-Gb3 levels are expected to drop. One dosing regimen for Fabry disease is administering to the patient about 100 mg to about 300 mg FBE of migalastat or salt thereof at a frequency of once every other day.

Over time, the levels of lyso-Gb3 may rise which can be due to either disease progression and/or decreasing ability of the kidneys to clear migalastat from the patient's body. Lyso-Gb3 levels will rise when the level of migalastat is too high because at higher levels the migalastat acts as an inhibitor of α-Gal A, thus preventing the enzyme from binding to the target substrate. Individuals with normal kidney function will generally clear a 150 mg dose of migalastat hydrochloride by 48 hours (i.e., C₄₈ₕ to below a level of quantification of about 5 ng/mL). In cases of severe kidney impairment, C₄₈ₕ may be 250 or even above 300 ng/mL. It is thought that high levels of migalastat are due to impaired kidney function because migalastat does not have other known interactions that would otherwise result in high levels.

Accordingly, another aspect of the invention pertains to method for treatment of Fabry disease in a patient having renal impairment. In one or more embodiments, the method comprises administering to the patient about 100 mg to about 300 mg FBE of migalastat or salt thereof at a first frequency of once every other day for a first time period; and administering to the patient about 100 mg to about 300 mg FBE of migalastat or salt thereof at a longer dosing interval (e.g., once every three to seven days) for a second time period. In some embodiments, the dosing frequency is adjusted after measuring lyso-Gb3 and/or migalastat levels. In some embodiments, the dosing frequency is adjusted after a change in the patient's kidney function (e.g. eGFR). For example, the dosing frequency can be adjusted as the patient's eGFR indicates a change from mild renal impairment to moderate renal impairment or a change from moderate renal impairment to severe renal impairment.

In some embodiments, the migalastat or salt thereof is administered at a first frequency for a first time period, and then administered at a second frequency for a second time period. The first frequency is greater (i.e., more frequent) than the second frequency. The first frequency and the second frequency may be any dosing interval disclosed herein. In some embodiments, the first frequency is every other day and the second frequency is every three days, every four days, every five days, every six days or every seven days. In some embodiments, the first frequency is every four days and the second frequency is every five days, every six days, or every seven days.

In some embodiments, the migalastat or salt thereof is administered at a first frequency for a first time period, then administered at a second frequency for a second time period, and then administered at a third frequency for a third time period. The first frequency is greater (i.e., more frequent) than the second frequency, and the second frequency is greater than the third frequency. For example, in some embodiments, the migalastat or salt thereof is administered at a first frequency of once every other day for a first time period, then the migalastat or salt thereof is administered at a second frequency of once every four days for a second time period, and then the migalastat or salt thereof is administered at a third frequency of once every seven days for a third time period.

In some embodiments, the dosing frequency is adjusted in response to a reduction in the patient's eGFR. In exemplary embodiments, when the patient's eGFR is reduced below 30 mL/min/1.73 m², the dosing frequency can be adjusted from every other day to every four days. In exemplary embodiments, when the patient's eGFR is reduced below 20 mL/min/1.73 m², the dosing frequency can be adjusted from every four days to every seven days. Other adjustments in dosing frequency can be made from one dosing interval to a longer dosing interval as described above. In some embodiments, the patient suffers from severe renal impairment.

In some embodiments, the method further comprises measuring migalastat levels. In one or more embodiments, migalastat concentration (e.g., ng/mL) is measured. In some embodiments, the total area under the curve (AUC_{0-∞}) is measured. In one or more embodiments, the lowest concentration the migalastat reaches before the next dose (C_{trough}) is measured. C_{trough} for QOD will be the concentration at 48 hours (C₄₈ₕ) C_{trough} for Q4D will be the concentration at 96 hours (C₉₆). Similarly, C_{trough} for Q7D will be the concentration at 168 hours (C₁₆₈). In one or more embodiments, the targeted C_{trough} values are at or near below the level of quantitation (BLQ). Such C_{trough} values indicate that the migalastat is being cleared from the body at an appropriate rate (i.e., is almost completely cleared before administration of the next dose).

Migalastat levels can be measured via methods known in the art. For example, if measuring migalastat from tissue samples, tissue aliquots may be homogenized (7 μL water per 1 mg tissue) using a homogenizer (e.g., FastPrep-24 from MP Biomedical, Irvine, Calif.). Microcentrifuge tubes containing 100 μl of the tissue homogenate or 50 μl of plasma may then be spiked with 500 ng/mL 13C d2-AT1001 HCl internal standard (manufactured by MDS Pharma Services). A 600 μl volume of 5 mM HCl in 95/5 MeOH:H₂O can then be added and the tubes vortexed for 2 minutes, followed by centrifugation at 21000×g for 10 minutes at room temperature. The supernatants may then be collected into a clean, 96-well plate, diluted with 5 mM HCl in dH₂O and applied to a 96-well solid phase extraction (SPE) plate (Waters Corp., Milford Mass.). After several wash steps and elution into a clean, 96-well plate, the extracts may be dried down under N₂ and reconstituted with mobile phase A. Migalastat levels can then be determined by liquid chromatography—tandem mass spectroscopy (LC-MS/MS) (e.g., LC: Shimadzu; MS/MS: ABSciex API 5500 MS/MS). The liquid chromatography can be conducted using an ACN:water:formate binary mobile phase system (mobile phase A: 5 mM ammonium formate, 0.5% formic acid in 95:5 ACN:water; mobile phase B: 5 mM ammonium formate, 0.5% formic acid in 5:47.5:47.5 ACN:MeOH:water) with a flow rate of 0.7 mL/minute on an Halo HILIC column (150×4.6 mm, 2.7 μm) (Advanced Materials Technology, Inc.). MS/MS analysis may be carried out under APCi positive ion mode. The same procedure may be followed for migalastat determination in plasma except without homogenization. The following precursor ion→product ion transitions may be monitored: mass/charge (m/z) 164.1→m/z 80.1 for migalastat and m/z 167.1→m/z 83.1 for the internal standard. A 12-point calibration curve and quality control samples may be prepared. The ratio of the area under the curve for migalastat to that of the internal standard is then determined and final concentrations of migalastat in each sample calculated using the linear least squares fit equation applied to the calibration curve. To derive approximate molar concentrations, one gram of tissue may be estimated as one mL of volume.

Migalastat concentration can be measured from plasma samples at various times to monitor clearance from the body. A clinically relevant increase in C_{trough} suggests significant accumulation of plasma migalastat concentration. If the migalastat is not cleared from the body enough prior to the next dose administration, then the levels of migalastat can build up, possibly leading to an inhibitory effect. Thus, in one or more embodiments, a change in the dosing frequency occurs after a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0-fold increase in C_{trough} compared to normal renal function C_{trough}. In one or more embodiments, the C_{trough} of normal renal function at is BLQ. In some embodiments, BLQ is 5 ng/mL of migalastat. A person with normal kidney function will generally clear 150 mg of migalastat HCl in 48 hours. Thus, a patient that is currently on a dosing QOD regimen of 150 mg of migalastat HCl should reach BLQ by 48 hours, which is also the C_{trough} value. If values above BLQ are measured at 48 hours in a patient on a QOD dosing regimen, then this may indicate a need to change the dosing interval. Accordingly, in one or more embodiments, the C_{trough} value of a patient with renal impairment (C₄₈ₕ if on a QOD regimen, C₉₆ if on a Q4D regimen or C₁₆₈ if on a Q7D regimen) will be compared with C_{trough} of a person with normal renal function (C₄₈ₕ).

In one or more embodiments, a change in the dosing frequency occurs after a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0-fold increase in AUC_{0-∞} compared to normal renal function AUC_{0-∞}.

In some embodiments, samples may be taken at 0, 1, 2, 3, 4, 6, 8, 12, 24, 48, 72, 96, 120, 144 and/or 168 hours after administration. In some embodiments, the migalastat concentration 48 hours after administration is measured. In some embodiments, the administration of the second time period is begun after more than about 5, 10, 15, 20, 25, 40, 50, 60, 70, 80, 90, 100, 125, 150, 175 or 200 ng/mL of migalastat is measured 48 hours after administration of the migalastat during the first time period is measured.

In further embodiments, the method further comprises measuring lyso-Gb3 in one or more plasma samples from the patient. A first baseline lyso-Gb3 level may be determined during the first time period. As used herein, “baseline lyso-Gb3 level” refers to the lowest plasma lyso-Gb3 value measured during a given time period or dosing regimen. Thus, if the lyso-Gb3 levels go up significantly from the baseline lyso-Gb3 levels, this may indicate kidney disease progression and/or improper clearance of migalastat. Thus, in further embodiments, the administration of the second time period is begun after an increase (e.g., of at least about 20, 25, 30, 33, 35, 40, 45 or 50% and/or 1, 1.25, 1.5, 1.75, 2, 2.25, 2.5 or 3 nM) above the first baseline lyso-Gb3 level is measured. A 33% and/or 2 nM increase from baseline in plasma lyso-Gb3 has been deemed clinically relevant based upon Phase 3 data in Fabry patients signaling either inhibition-induced migalastat exposure from decline in renal function and/or progression of disease condition. Lyso-Gb3 levels may be measured at varying frequencies (e.g., about once every 2, 3, 4 or 5 months). It is thought that it takes about 3 months for a baseline lyso-Gb3 level to be established once a dosing regimen has been started.

Lyso-Gb3 can be measured via methods known in the art using validated assays. As with migalastat, lyso-Gb3 levels may be determined using liquid chromatography—tandem mass spectroscopy (LC-MS/MS) (e.g., LC: Shimadzu; MS/MS: ABSciex API 5500 MS/MS). For example, one process of measuring plasma lyso-Gb3 is described in Hamler, Rick, et al. “Accurate quantitation of plasma globotriaosylsphingosine (lyso-Gb3) in normal individuals and Fabry disease patients by liquid chromatography—tandem mass spectrometry (LC—MS/MS).” Molecular Genetics and Metabolism, Volume 114.2 (2015):S51. In one or more embodiments, lyso-Gb3 is measured in samples from a patient's urine.

Thus, in one exemplary embodiment, the method comprises
administering to the patient about 100 mg to about 300 mg FBE of migalastat or salt thereof at a first frequency of once every other day for a first time period;
administering to the patient about 100 mg to about 300 mg FBE of migalastat or salt thereof at a second frequency of once every four or seven days for a second time period;
measuring lyso-Gb3 in one or more plasma samples from the patient;
determining a first baseline lyso-Gb3 level during the first time period;
measuring migalastat concentration, AUC_{0-∞} and/or C_{trough} in one or more plasma samples during the first time period; and
beginning the administration at the second frequency after
   (i) an increase above the first baseline lyso-Gb3 level, and/or
   (ii) more than about 5 ng/mL of migalastat is measured 48 hours after administration of the migalastat during the first time period, or there is a greater than 1.5-fold increase in AUC_{0-∞} and/or C_{trough} compared to normal renal function during the first time period.

In further embodiments, the administration of the second time period may begin after an increase above the first baseline lyso-Gb3 level is at least about 30, or 33% and/or 2 nM and/or more than about 50 ng/mL of migalastat is measured 48 hours after administration of the migalastat during the first time period is measured. In some embodiments, the administration of the second time period may begin after an increase above the first baseline lyso-Gb3 level is at least about 30, or 33% and/or 2 nM and/or more than about 50 ng/mL of migalastat is measured 48 hours after administration of the migalastat during the first time period is measured, or there is a greater than 1.5-fold increase in AUC_{0-∞} and/or C_{trough} compared to normal renal function during the first time period.

In further embodiments, dosing from every other day is adjusted to every four days, and then further adjusted to every seven days. In such embodiments, the frequency of the second time period is once every four days, and the method further comprises administering to the patient about 100 mg to about 300 mg FBE of migalastat or salt thereof at a third frequency of once every seven days for a third time period. In yet further embodiments, the method may further comprise
measuring lyso-Gb3 in one or more plasma samples from the patient;
determining a first baseline lyso-Gb3 level during first time period;
measuring migalastat concentration, AUC_{0-∞} and/or C_{trough} in one or more plasma samples from the patient during the first time period;
beginning the administration of the second time period after
   (i) an increase above the first baseline lyso-Gb3 level, and
   (ii) more than about 5 ng/mL of migalastat is measured 48 hours after administration of the migalastat during the first time period; or there is a greater than 1.5-fold increase in AUC_{0-∞} and/or C_{trough} compared to normal renal function during the first time period
determining a second baseline lyso-Gb3 level during the second time period; and
beginning the administration of the third time period after
   (i) an increase above the second baseline lyso-Gb3 level, and
   (ii) more than about 5 ng/mL of migalastat is measured 48 hours after administration of the migalastat during the second time period, or there is a greater than 1.5-fold increase in AUC_{0-∞} and/or C_{trough} compared to normal renal function during the first time period.

In other embodiments, the dosing from every other day is adjusted directly to every seven days without first adjusting to four days.

In an exemplary embodiment, a Fabry patient may be receiving 150 mg of migalastat HCl every other day. If upon measuring plasma lyso-Gb3 levels, the following are measured: (1) an increase in plasma lyso-Gb3 relative to the baseline level for the current dose regimen (e.g., at least a 30 or 33% increase); and/or (2) an increase of at least 2 nM in plasma lyso-Gb3 relative to the baseline level for the current dose regimen, the dosing regimen may be changed to once every four or seven days. If the patient's migalastat levels are also high, then the regimen may also be changed to once every four or seven days. Such high levels of migalastat could be a measurement of the AUC_{0-∞} and/or C_{trough} that is higher compared to normal renal function during the first time period (e.g., 1.5 or 2-fold increase).

Once the dose regimen has been changed, a new plasma lyso-Gb3 baseline level will be established. Any new dose regimen modifications will be based on a comparison to the subject's most current baseline level. For example, a new baseline level may be established as follows: if a subject has a decrease in plasma lyso-Gb3 relative to their previous measurement, a confirmatory retest may take place. If the confirmatory value is also lower than their previous measurement, the average of the 2 values will be the subject's new baseline level. If the retest is NOT lower than the subject's previous measurement, the previous measurement will continue as the current baseline level until the next visit.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in various embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

Patents, patent applications, publications, product descriptions, and protocols are cited throughout this application, the disclosures of which are incorporated herein by reference in their entireties for all purposes.

### EXAMPLES

### Example 1: Pharmacokinetics of Migalastat in Non-Fabry Patients with Renal Impairment

A phase 1 trial was conducted to study the pharmacokinetics and safety of migalastat HCl in non-Fabry subjects with renal impairment. The results are reported in Johnson, et al. “An Open-Label Study to Determine the Pharmacokinetics and Safety of Migalastat HCl in Subjects With Impaired Renal Function and Healthy Subjects with Normal Renal Function.” American College of Clinical Pharmacology 4.4 (2015): 256-261, and is also described here. A single 150 mg migalastat HCl dose was administered to subjects with mild, moderate, and severe renal impairment, and normal renal function. The eGFR estimated by the Cockcroft-Gault equation per the FDA Guidance for renal impairment studies.

Volunteers were enrolled into two cohorts stratified for renal function calculated using the Cockcroft-Gault equation for creatinine clearance (CLcr). Subjects were assigned to groups based on an estimated CLcr at screening as calculated using the Cockcroft-Gault equation. For each subject, the following plasma migalastat PK parameters were determined by noncompartmental analysis with WinNonlin® software (Pharsight Corporation, Version 5.2).
Cₘₐₓ maximum observed concentration
tₘₐₓ time to maximum concentration
AUC₀₋ₜ area under the concentration-time curve from Hour 0 to the last measurable concentration, calculated using the linear trapezoidal rule for increasing concentrations and the logarithmic rule for decreasing concentrations AUC_{0-∞} area under the concentration-time curve extrapolated to infinity, calculated using the formula:

AUC_{0-∞}=AUC0-t+Ct/λZ
where Ct is the last measurable concentration and λZ is the apparent terminal elimination rate constant
λz apparent terminal elimination rate constant, where λZ is the magnitude of the slope of the linear regression of the log concentration versus time profile during the terminal phase
t_{1/2} apparent terminal elimination half-life (whenever possible), where
t½=(ln2)/λZ
CL/F oral clearance, calculated as Dose/AUC0-∞·λZ
V_{d}/F oral volume of distribution, calculated as Dose/AUC0-∞·λZ
C₄₈ₕ concentration at 48 hours postdose

Pharmacokinetic parameters determined were: area under the concentration—time curve (AUC) from time zero to the last measurable concentration postdose (AUC₀₋ₜ) and extrapolated to infinity (AUC_{0-∞}), maximum observed concentration (Cₘₐₓ), time to Cₘₐₓ(tₘₐₓ), concentration at 48 hours postdose (C₄₈ₕ), terminal elimination half-life (t_{1/2}), oral clearance (CL/F), and apparent terminal elimination rate constant (λz) (ClinicalTrials.gov registration: NCT01730469).

Study subjects were defined as having renal impairment if creatinine clearance (CLcr) was less than 90 mL/min (i.e. CLcr <90 mL/min) as determined using the Cockcroft-Gault formula. Subjects were grouped according to degree of renal dysfunction: mild (CLcr ≥60 and <90 mL/min), moderate (CLcr ≥30 and <60 mL/min), or severe (CLcr ≥15 and <30 mL/min)

The plasma and urine pharmacokinetics of migalastat have been studied in healthy volunteers and Fabry patients with normal to mildly impaired renal function. In the single-dose studies, migalastat had a moderate rate of absorption reaching maximum concentrations in approximately 3 hours (range, 1 to 6 hrs) after oral administration over the dose range studied. Mean Cₘₐₓ and AUC0-t values increased in a dose-proportional manner following oral doses from 75 mg to 1250 mg migalastat. The mean elimination half-lives (t1/2) ranged from 3.04 to 4.79 hours. Mean percent of the dose recovered in urine from doses evaluated in the single ascending dose (SAD) study were 32.2%, 43.0%, 49.3%, and 48.5% for the 25 mg, 75 mg, 225 mg, and 675 mg dose groups, respectively. In multiple ascending dose studies, only minimal accumulation of plasma migalastat was observed. In a TQT study, migalastat was negative for effect on cardiac repolarization at 150 mg and 1250 mg single doses (Johnson et al. “Pharmacokinetics and Safety of Migalastat HCl and Effects on Agalsidase Activity in Healthy Volunteers.” Clin Pharmacol Drug Dev. 2013 April; 2(2):120-32 2013).

In this single dose renal impairment study conducted in non-Fabry subjects, plasma concentrations of single-dose migalastat HCl 150 mg increased with increasing degree of renal failure compared to subjects with normal renal function. Following a single oral dose of migalastat HCl 150 mg, mean plasma migalastat AUC_{0-∞} increased in subjects with mild, moderate, or severe renal impairment by 1.2-fold, 1.8-fold, and 4.5-fold, respectively, compared to healthy control subjects. Increases in plasma migalastat 150 mg AUC_{0-∞} values were statistically significant in subjects with moderate or severe renal impairment but not in subjects with mild renal impairment following single-dose administration compared to subjects with normal renal function. Migalastat tmax was slightly delayed in the severe group; Cₘₐₓ was not increased across any of the groups following a single oral dose of migalastat HCl 150 mg in subjects with varying degrees of renal impairment compared to healthy control subjects. Plasma migalastat C₄₈ₕ levels were elevated in subjects with moderate (predominantly from subjects with CrCL <50 ml/min) and severe renal impairment compared with healthy control subjects. The t_{1/2} of migalastat in plasma increased as the degree of renal impairment increased (arithmetic mean [min, max]: 6.4 [3.66, 9.47], 7.7 [3.81, 13.8], 22.2 [6.74, 48.3], and 32.3 [24.6, 48.0] h) in subjects with normal renal function and those with mild, moderate, or severe renal impairment, respectively. Mean CL/F decreased with increasing degree of renal failure and ranged from 12.1 to 2.7 L/hr from mild to severe renal impairment (Johnson et al. 2014).

Migalastat clearance decreased with increasing renal impairment, resulting in increases in migalastat HCl plasma t_{1/2}, AUC_{0-∞}, and C₄₈ₕ compared with subjects with normal renal function. Incidence of adverse events was comparable across all renal function groups.

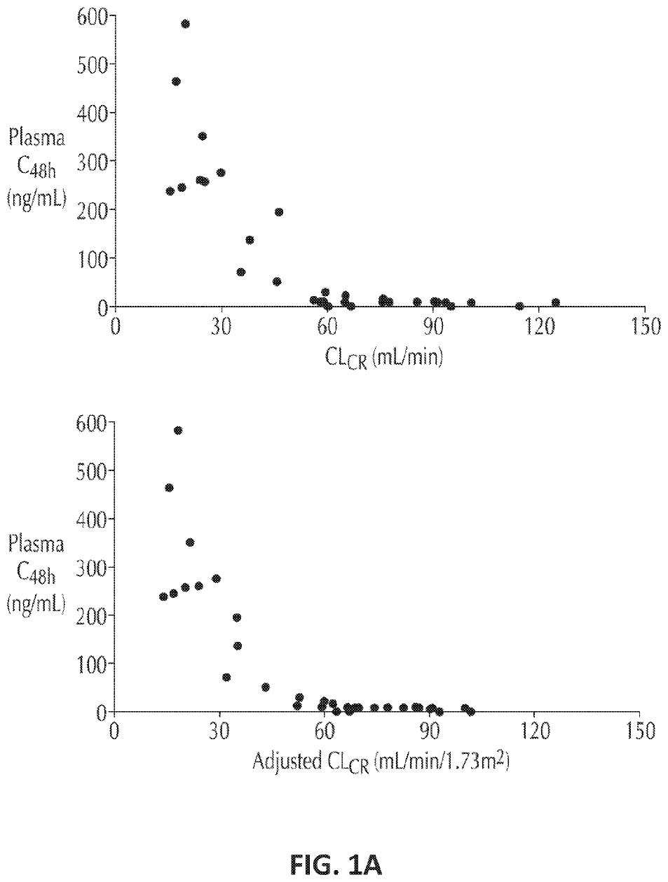

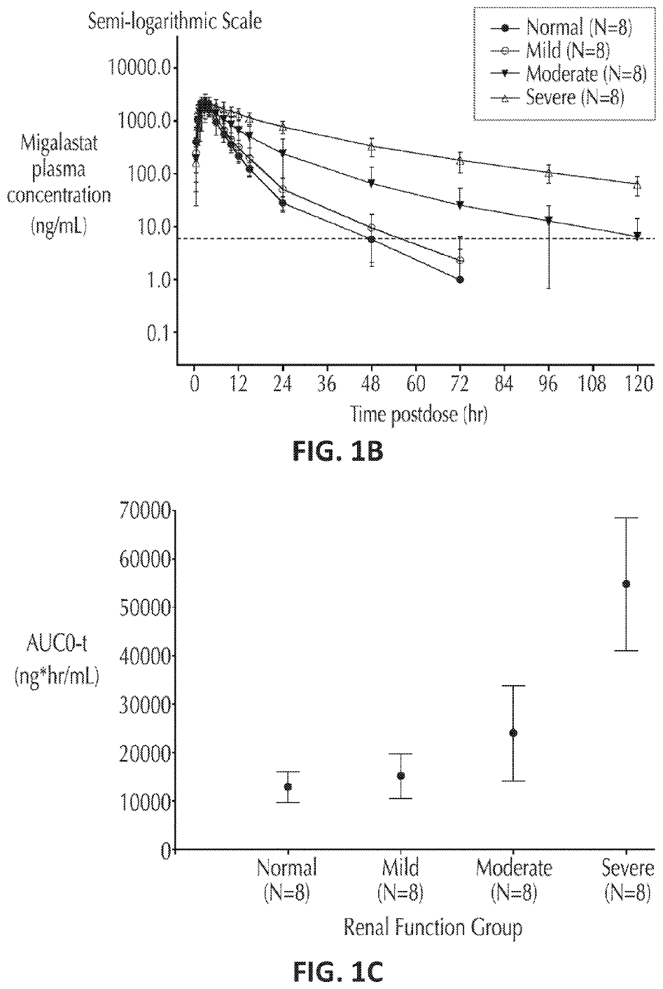

Following a single oral dose of 150 mg migalastat HCl plasma exposure (expressed as AUC₀₋ₜ) increased as the degree of renal impairment increased. FIG. 1A shows an increase in migalastat AUC₀₋ₜ values as CLcr values decrease. FIG. 1B shows the mean (SE) plasma migalastat concentration-time profiles for each renal function group. BLQ values were entered as zero and included in the calculation of means.

As demonstrated in FIG. 1C, as renal impairment worsens, plasma migalastat AUC₀₋ₜ values increase in a nonlinear manner. Results demonstrated that, as renal impairment worsened, the clearance of plasma migalastat decreased, resulting in prolonged t_{1/2}, higher C₄₈ₕ values, and higher overall plasma exposure (AUC_{0-∞}), in particular in subjects with severe renal impairment. Migalastat is primarily excreted unchanged in urine. Thus, an increase in plasma migalastat exposure is consistent with worsening renal impairment.

Conclusions: Plasma migalastat clearance decreased as degree of renal impairment increased

A summary of the PK results are shown in Table 3 below.

TABLE 3

| | | Renal Function Group | | | | | | | |
|---|---|---|---|---|---|---|---|---|---|
| PK | | Normal | | Mild | | Moderate | | Severe | |
| Parameter | Units | (N = 8) | | (N = 8) | | (N = 8) | | (N = 8) | |
| AUC₀₋ₜ | (ng · hr/mL) | 12306 | (27.9) | 14389 | (31.1) | 22126 | (42.8) | 53070 | (27.0) |
| AUC_{0-∞} | (ng · hr/mL) | 12397 | (27.7) | 14536 | (30.7) | 22460 | (42.2) | 56154 | (24.9) |
| Cₘₐₓ | (ng/mL) | 2100 | (26.0) | 2191 | (28.8) | 1868 | (32.1) | 2078 | (45.5) |
| tₘₐₓ | (hr) | 2.50 | (1.50, 3.00) | 2.50 | (1.50. 4.00) | 3.00 | (1.50, 4.00) | 4.27 | (3.00, 8.00) |
| t_{1/2} | (hr) | 6.42 | (1.93) | 7.66 | (3.02) | 22.2 | (14.2) | 32.3 | (7.35) |
| CL/F | (L/hr) | 12.1 | (27.7) | 10.3 | (30.7) | 6.68 | (42.2) | 2.67 | (24.9) |
| C_{48 h} | (ng/mL) | 5.70 | (3.63) | 9.34 | (7.57) | 64.5 | (68.1) | 334 | (126) |

### Example 2: Multiple Dose Simulations on Renal Impairment Subjects

In the renal impairment study of Example 1, consistent increases in area under the curve (AUC) and trough concentration of migalastat at 48 hours post-dose following QOD dosing (C₄₈ₕ) of 2- to 4-fold were observed at eGFR values <35 mL/min relative to subjects with normal renal function.

A population PK model was developed to predict exposures and time above IC50 in Fabry patients with varying degrees of renal impairment. Various dosing regimens were assessed to develop an understanding of migalastat exposure in patients with different ranges of renal impairment (≥30, 20-30, <20 mL/min/1.73 m²). The dosing regimens evaluated included 150 mg every other day (QOD), 150 mg every 4 days (Q4D), and 150 mg once weekly (Q7D).

A model-based dose finding approach was used to predict appropriate migalastat dosing in a Fabry patient sub-population, namely Fabry patients with renal impairment. In general, dose optimization goals to which model-based drug development (MBDD) methods can be applied include: (1) predicting first-in-human dose; (2) finding the dose or dose range that best balances safety and efficacy; (3) finding best dose frequency; (4) finding promising combinations for co-administered drugs; (5) accounting for realistic subject behavior, including adherence; (6) maximizing early phase learning to strengthen dose confirmation.

This example provides computer simulations of dosing the renal impairment subjects of Example 1. The key assumption was exposure characterized in non-Fabry subjects with renal impairment is the same as in Fabry patients with renal impairment. The software program was WinNonlin version 5.2 or higher. The conditions of the model are described below. 11 subjects who had BSA-adjusted eGFR_{Cockcroft-Gault}≤35 mL/min/1.73 m² were included in the modeling exercise; 3 had moderate renal impairment, but were ≥30 mL/min/1.73 m² and ≤35 mL/min/1.73 m², and 8 were ≥14 mL/min/1.73 m² and <30 mL/min/1.73 m². Steady state was assumed by 7^{th} dose.

Four regimens with 150 mg migalastat HCl were simulated: QOD (every other day or 48 hrs), Q3D (every 3^{rd} day or 72 hrs), Q4D (every 4^{th} day or 96 hrs), and Q7D (every 7^{th} day/once a week or 168 hrs).

A 2-compartment model was used to estimate volume of distribution (V_{d}) and elimination rate constants from single dose data. These estimates were inputted into each molecular dose simulation regimen.

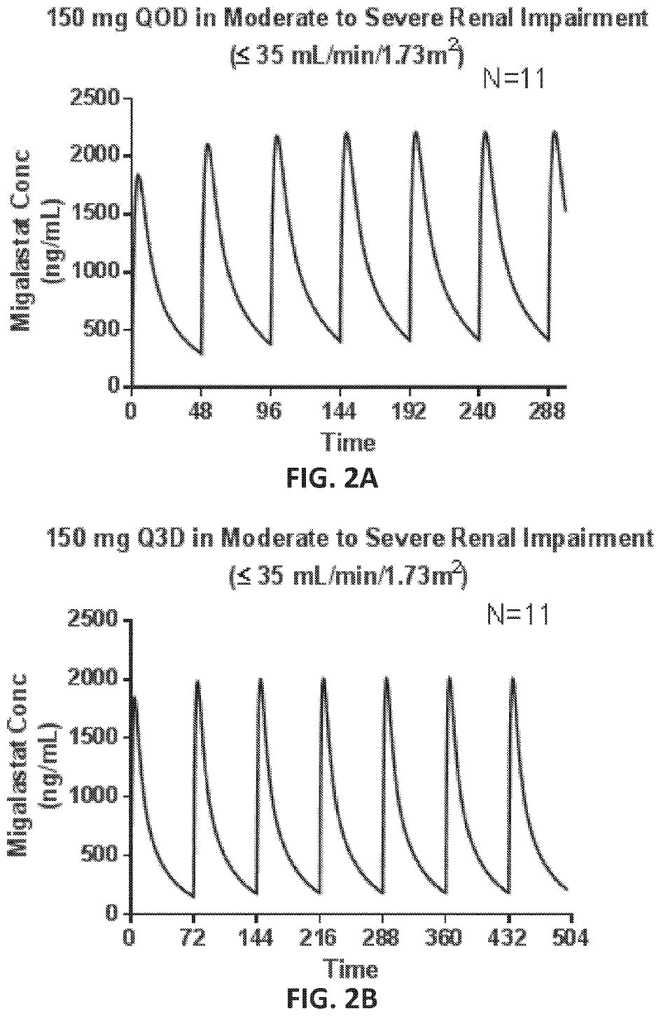

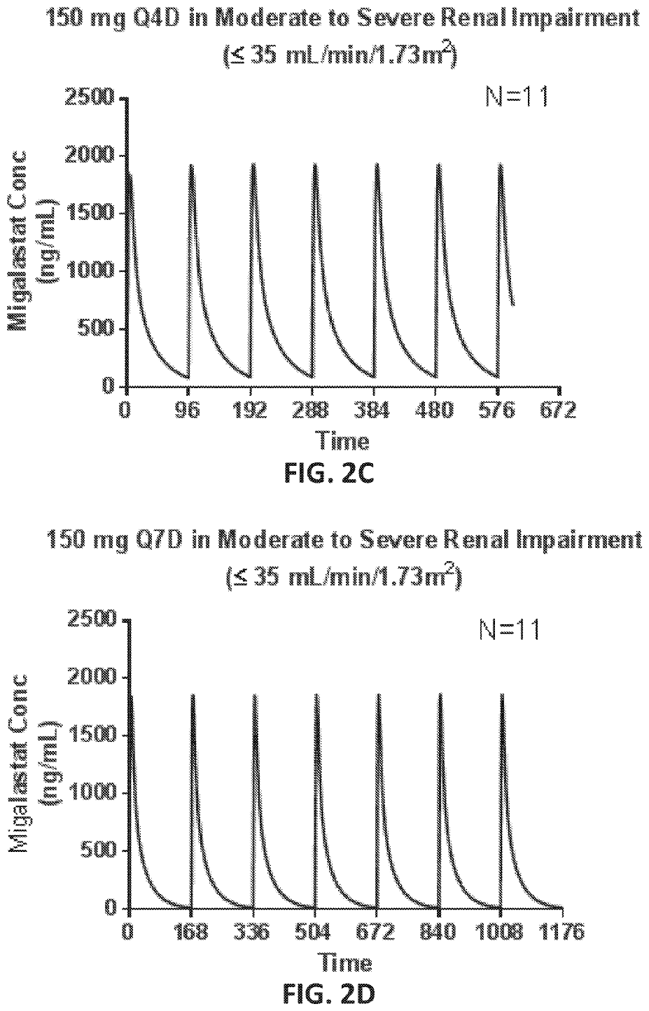

FIGS. 2A-D show the mean simulation plots for each regimen. Table 4 below shows the exposures and accumulation ratios. Based on AUCs, MD simulations suggest accumulation is minimal (≤5%) for Q7D dosing. The highest exposure of migalastat in a Fabry patient was recorded as 53035 ng*hr/mL, who received a single dose of 450 mg.

Table 5 below shows the C_{min,ss} for a 150 mg regimen. Based on C_{min,ss}, MD simulations for Q7D are similar to PPK Cₘᵢₙ (8.70 ng/mL) for most subjects.

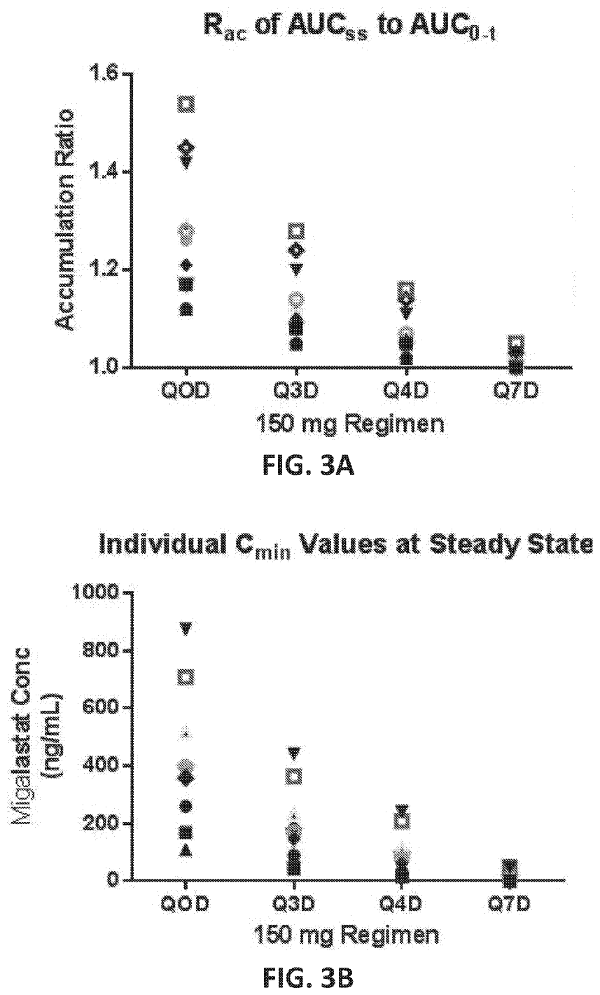

FIGS. 3A-B show the R_{ac} and Cₘᵢₙ values across simulated regimens. FIG. 3A shows the QOD regimen has greater accumulation of migalastat, then Q3D, Q4D lesser yet, and Q7D has virtually none in severe renal impairment. FIG. 3B also shows this trend, but for C₄₈ₕ concentration.

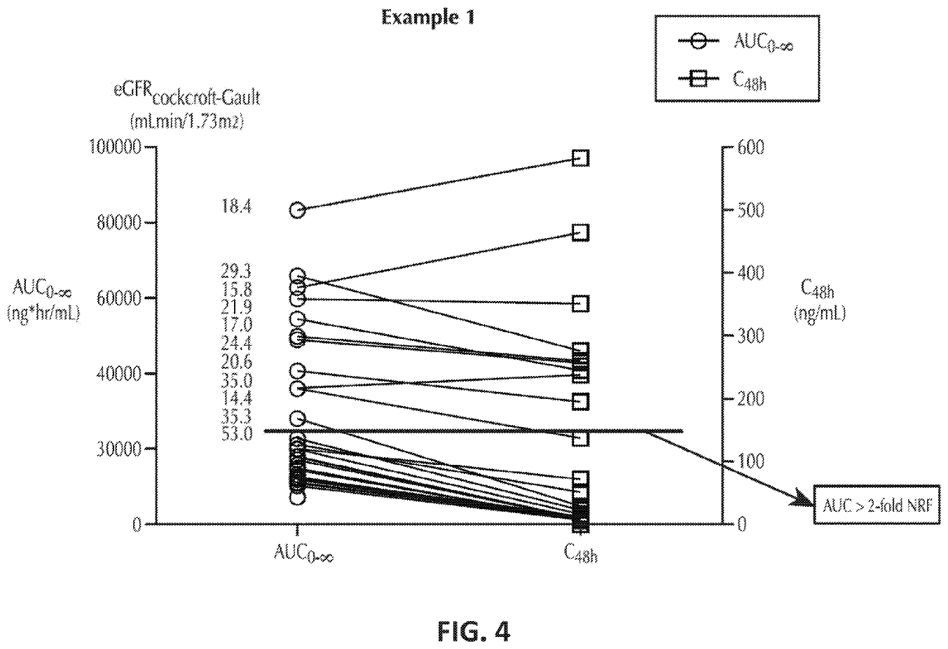

FIG. 4 shows AUC versus C₄₈ₕ from Example 1. This stick plot provides a visual correlation of AUC to C₄₈ₕ concentration across all levels of renal function, and demonstrates the two values are well visually correlated.

Tables 6-7 are provided below showing a summary of the population PK modeling and time above IC₅₀ (inhibition).

Based on predicted exposure data, clinical trial simulations suggest that a Q4D regimen would provide exposures similar to subjects with normal renal function for those with eGFR between >30 and <40 mL/min/1.73 m² and a Q7D regimen would provide exposures similar to subjects with normal renal function for those with eGFR between >20 and <30 mL/min/1.73 m².

This modelling predicts the slower removal of migalastat based upon the level of kidney impairment and adjusts the frequency of dosing to bring the level of migalastat below the level where it would inhibit enzyme activity.

Simulated migalastat exposures following Q7D in subjects with eGFR <20 mL/min/1.73 m² remained 5- to 6 fold higher than those with normal renal function.

TABLE 4

| | | BSA-Adj | | | | | |
|---|---|---|---|---|---|---|---|
| | Renal Function | eGFR_{Cockcroft-Gault} | | | | | |
| SubjID | Group | (mL/min/1.73 m²) | AUC_{0-48 h} | R_{ac48 h} | AUC_{0-72 h} | R_{ac72 h} | AUC_{0-96 h} |
| 10-104 | Moderate (≥30-≤35) | 35.3 | 31920 | 1.12 | 34058 | 1.05 | 34978 |
| 9-102 | Moderate (≥30-≤35) | 35.0 | 35320 | 1.17 | 38364 | 1.08 | 39608 |
| 5-306 | Moderate (≥30-≤35) | 32.2 | 17507 | 1.12 | 18670 | 1.05 | 19211 |
| 25-419 | Severe (<30) | 18.4 | 59178 | 1.42 | 69646 | 1.20 | 75485 |
| 26-111 | Severe (<30) | 17.0 | 44124 | 1.21 | 48296 | 1.10 | 50597 |
| 27-331 | Severe (<30) | 20.6 | 37409 | 1.28 | 41912 | 1.14 | 44571 |
| 28-113 | Severe (<30) | 15.8 | 41687 | 1.54 | 50314 | 1.28 | 55234 |
| 29-213 | Severe (<30) | 21.9 | 45790 | 1.29 | 52219 | 1.13 | 55599 |
| 30-214 | Severe (<30) | 29.3 | 56331 | 1.17 | 61017 | 1.08 | 63406 |
| 31-215 | Severe (<30) | 14.4 | 23732 | 1.45 | 27882 | 1.24 | 30325 |
| 32-114 | Severe (<30) | 24.4 | 39012 | 1.26 | 43768 | 1.12 | 46504 |

| | | | | | | |
|---|---|---|---|---|---|---|
| Geometric Mean | 22.9 | 37256 | 1.27 | 41756 | 1.13 | 44156 |
| CV % | 33.8 | 33.4 | 11.1 | 34.8 | 6.7 | 35.7 |

| | Renal Function | | | | | | | |
|---|---|---|---|---|---|---|---|---|
| SubjID | Group | R_{ac96 h} | AUC_{0-168 h} | R_{ac168 h} | AUC_{PPK} | R_{acPPK} | AUCₛₛ | AUC_{0-∞} |
| 10-104 | Moderate (≥30-≤35) | 1.02 | 35487 | 1.00 | 9515 | 3.75 | 35716 | 36051 |
| 9-102 | Moderate (≥30-≤35) | 1.05 | 41252 | 1.00 | 9515 | 4.36 | 41475 | 40653 |
| 5-306 | Moderate (≥30-≤35) | 1.02 | 19460 | 1.00 | 9515 | 2.06 | 19560 | 19723 |
| 25-419 | Severe (<30) | 1.11 | 81332 | 1.03 | 9515 | 8.81 | 83805 | 83312 |
| 26-111 | Severe (<30) | 1.05 | 52861 | 1.00 | 9515 | 5.59 | 53212 | 54470 |
| 27-331 | Severe (<30) | 1.07 | 47263 | 1.01 | 9515 | 5.02 | 47769 | 48890 |
| 28-113 | Severe (<30) | 1.16 | 61478 | 1.05 | 9515 | 6.74 | 64157 | 62034 |
| 29-213 | Severe (<30) | 1.07 | 58554 | 1.01 | 9515 | 6.23 | 59238 | 59538 |
| 30-214 | Severe (<30) | 1.04 | 65284 | 1.00 | 9515 | 6.91 | 65750 | 65916 |
| 31-215 | Severe (<30) | 1.14 | 33445 | 1.03 | 9515 | 3.62 | 34459 | 36227 |
| 32-114 | Severe (<30) | 1.06 | 48776 | 1.01 | 9515 | 5.17 | 49167 | 49905 |

| | | | | | | | |
|---|---|---|---|---|---|---|---|
| Geometric Mean | 1.07 | 46541 | 1.01 | 9515 | 4.96 | 47238 | 47570 |
| CV % | 4.3 | 36.8 | 1.7 | 0.0 | 37.5 | 37.5 | 36.4 |

NCA SD Data
Sim2-C Model
011 [symbol] PK model [symbol]
indicates data missing or illegible when filed

TABLE 5

| | | BSA-Adj | | | QOD | | | Q3D | |
|---|---|---|---|---|---|---|---|---|---|
| | Renal Function | eGFR_{Cockcroft-Gault} | AUCₛₛ | C_{max, SD} | C_{min, SD} | C_{max, SS} | C_{min, SS} | C_{min, SD} | C_{max, SS} |
| SubjID | Group | (mL/min/1.73 m²) | (ng*hr/mL) | (ng/mL) | (ng/mL) | (ng/mL) | (ng/mL) | (ng/mL) | (ng/mL) |
| 9-102 | Moderate (>=30-<60) | 35.0 | 41475 | 1899 | 223 | 2122 | 260 | 86.0 | 1976 |
| 10-104 | Moderate (>=30-<60) | 35.3 | 35716 | 1967 | 155 | 2111 | 169 | 48.0 | 2008 |
| 5-306 | Moderate (>=30-<60) | 32.2 | 19560 | 882 | 87.8 | 971 | 109 | 39.1 | 916 |
| 25-419 | Severe (<30) | 18.4 | 83805 | 2331 | 562 | 3086 | 876 | 344 | 2719 |
| 26-111 | Severe (<30) | 17.0 | 53212 | 2329 | 294 | 2646 | 368 | 134 | 2454 |
| 27-331 | Severe (<30) | 20.6 | 47769 | 1953 | 297 | 2306 | 395 | 150 | 2105 |
| 28-113 | Severe (<30) | 15.8 | 64157 | 1364 | 436 | 1923 | 709 | 267 | 1665 |
| 29-213 | Severe (<30) | 21.9 | 59238 | 1965 | 379 | 2404 | 518 | 200 | 2162 |
| 30-214 | Severe (<30) | 29.3 | 65750 | 3667 | 315 | 3999 | 381 | 134 | 3787 |
| 31-215 | Severe (<30) | 14.4 | 34459 | 900 | 229 | 1201 | 358 | 140 | 1056 |
| 32-114 | Severe (<30) | 24.4 | 49167 | 1729 | 312 | 2073 | 405 | 152 | 1873 |

| | | | | | | | | |
|---|---|---|---|---|---|---|---|---|
| Geometric Mean | 22.9 | 47238 | 1770 | 270 | 2114 | 358 | 129 | 1929 |
| CV % | 33.8 | 37.5 | 43.2 | 48.4 | 39.2 | 62.1 | 69.6 | 40.5 |

| | | Q3D | Q4D | | | Q7D | | |
|---|---|---|---|---|---|---|---|---|
| | Renal Function | C_{min, SS} | C_{min, SD} | C_{max, SS} | C_{min, SS} | C_{min, SD} | C_{max, SS} | C_{min, SS} |
| SubjID | Group | (ng/mL) | (ng/mL) | (ng/mL) | (ng/mL) | (ng/mL) | (ng/mL) | (ng/mL) |
| 9-102 | Moderate (>=30-<60) | 89.5 | 33.0 | 1927 | 33.3 | 1.88 | 1887 | 1.83 |
| 10-104 | Moderate (>=30-<60) | 48.9 | 14.9 | 1979 | 14.8 | 0.446 | 1967 | 0.431 |
| 5-306 | Moderate (>=30-<60) | 42.8 | 17.6 | 89.5 | 18.1 | 1.60 | 883 | 1.57 |
| 25-419 | Severe (<30) | 443 | 211 | 2545 | 243 | 48.7 | 2375 | 49.9 |
| 26-111 | Severe (<30) | 147 | 61.0 | 2381 | 62.9 | 5.79 | 2333 | 5.68 |
| 27-331 | Severe (<30) | 171 | 75.6 | 2022 | 79.9 | 9.74 | 1950 | 9.63 |
| 28-113 | Severe (<30) | 364 | 173 | 1540 | 209 | 48.0 | 1404 | 49.9 |
| 29-213 | Severe (<30) | 232 | 105 | 2059 | 113 | 15.5 | 1978 | 15.4 |
| 30-214 | Severe (<30) | 143 | 56.5 | 3714 | 57.5 | 4.31 | 3639 | 4.22 |
| 31-215 | Severe (<30) | 181 | 86.1 | 986 | 99.5 | 20.1 | 918 | 20.5 |
| 32-114 | Severe (<30) | 170 | 73.8 | 1795 | 76.9 | 8.51 | 1733 | 8.39 |

| | | | | | | | |
|---|---|---|---|---|---|---|---|
| Geometric Mean | 149 | 62.3 | 1501 | 66.5 | 7.01 | 1780 | 6.97 |
| CV % | 83.1 | 98.6 | 60.5 | 110.7 | 250.4 | 42.5 | 260.9 |

TABLE 6

| | AUC₀₋ₜ (ng*hr/mL) | | Cₘₐₓ (ng/mL) | | C_{trough} (ng/mL) | | C₄₈ (ng/mL) | |
|---|---|---|---|---|---|---|---|---|
| | Geometric mean (log SD) | | Geometric mean (log SD) | | Geometric mean (log SD) | | Geometric mean (log SD) | |
| Group | [% CV] (range) | | [% CV] (range) | | [% CV] (range) | | [% CV] (range) | |
| Normal | 9117 | (0.3) | 1372 | (0.34) | 5.8 | (0.64) | 5.8 | (0.64) |
| qod | [33.0%] | (5480-15382) | [34.9%] | (783-2395) | [71.6%] | (2.1-16.5) | [71.6%] | (2.1-16.5) |
| >44-60 mL/min/1.73 m² | 18099 | (0.3) | 1694 | (0.33) | 31.2 | (0.64) | 31.2 | (0.64) |
| qod | [33.7%] | (10581-30566) | [34.2%] | (978-2953) | [7008%] | (10.9-85) | [70.8%] | (10.9-85.0) |
| >35-40 mL/min/1.73 m² | 23270 | (0.3) | 1757 | (0.34) | 10 | (0.67) | 51.0 | (0.55) |
| q4d | [33.6%] | (13618-39906) | [35.4%] | (998-3125) | [75.6%] | (3.2-30.4) | [59.3%] | (20.2-123.4) |
| >35-40 mL/min/1.73 m² | 23077 | (0.3) | 1745 | (0.34) | 1.05 | (0.76) | 47.9 | (0.51) |
| q1w | [31.9%] | (8252-61784) | [34.6%] | (540-4776) | [86.4%] | (0.1-10.9) | [55.1%] | (9.8-205.4) |
| >=30-35 mL/min/1.73 m² | 26936 | (0.3) | 1820 | (0.34) | 14.9 | (0.67) | 73.7 | (0.53) |
| q4d | [33.7%] | (15631-45737) | [35.4%] | (1044-3195) | [74.9%] | (4.9-42.9) | [56.6%] | (29.6-166.2) |
| >=30-35 mL/min/1.73 m² | 26376 | (0.3) | 1781 | (0.34) | 1.63 | (0.76) | 68.0 | (0.49) |
| q1w | [32.1%] | (8723-73640) | [34.5%] | (581-4832) | [88.2%] | (0.14-20.4) | [52.5%] | (14.1-305.1) |
| >=20-24 mL/min/1.73 m² | 34599 | (0.3) | 1905 | (0.35) | 30.3 | (0.70) | 134.7 | (0.53) |
| q4d | [35.5%] | (19741-61742) | [35.6%] | (1084-3420) | [79.1%] | (9.5-97.3) | [56.6%] | (55.3-318.1) |
| >=20-29 mL/min/1.73 m² | 34115 | (0.3) | 1867 | (0.33) | 3.9 | (0.82) | 124.2 | (0.49) |
| q1w | [53.7%] | (19948-59335) | [34.1%] | (1098-3239) | [98.1%] | (1-15) | [52.1%] | (54.0-271.4) |
| <20 mL/min/1.73 m² | 55850 | (0.4) | 2105 | (0.35) | 107.9 | (0.69) | 350.2 | (0.49) |
| q4d | [33.3%] | (30143-101549) | [35.7%] | (1189-3709) | [78.5%] | (33-320.4) | [52.2%] | (151.3-752.8) |
| <20 mL/min/1.73 m² | 55150 | (0.4) | 2002 | (0.33) | 19.7 | (0.88) | 312.5 | (0.45) |
| q1w | [37.1%] | (30510-99679) | [34.3%] | (1152-3505) | [108%] | (4.4-80.2) | [47.4%] | (143.5-628.0) |
| <20 mL/min/1.73 m² | 55567 | (0.36) | 1987 | (0.34) | 0.54 | (1.38) | 307.1 | (0.43) |
| q2w | [37.4%] | (30271-99289) | [35.2%] | (1132-3468) | [240%] | (0.05-4.71) | [45.4%] | (145-598) |

TABLE 7

| | Time above Most Sensitive IC₅₀ (hr) | | Time above Least Sensitive IC₅₀ (hr) | |
|---|---|---|---|---|
| | Geometric mean (log SD) | | Geometric mean (log SD) | |
| Group | [% CV] (range) | | [% CV] (range) | |
| Normal qod | 12 .6 | (0.17) | 11.6 | (0.17) |
| | [17.4%] | (9.6-16.8) | [17.3%] | (8.9-15.5) |
| >40-60 mL/min/1.73 m² | 24.9 | (0.23) | 22.5 | (0.23) |
| qod | [23.7%] | (17.4-37.5) | [23.1%] | (15.8-33.3) |
| >35-40 mL/min/1.73 m² | 31.4 | (0.22) | 28.2 | (0.21) |
| q4d | [22.2%] | (22.5-46.3) | [21.3%] | (20.5-40.8) |
| >35-40 mL/min/1.73 m² | 30.7 | (0.20) | 27.6 | (0.19) |
| q1w | [19.9%] | (18.2-65.6) | [19.1%] | (16.7-56.7) |
| >=30-35 mL/min/1.73 m² | 36.9 | (0.23) | 33.0 | (0.23) |
| q4d | [23.7%] | (25.8-55.3) | [22.9%] | (23.4-48.3) |
| >=30-35 mL/min/1.73 m² | 35.6 | (0.21) | 31.9 | (0.20) |
| q1w | [21.4%] | (20.5-83.9) | [26.5%] | (18.2-74.0) |
| >=20-29 mL/min/1.73 m² | 49.7 | (0.28) | 44.2 | (0.28) |
| q4d | [28.3%] | (32.4-82.9) | [28.1%] | (29.1-73.5) |
| >=20-29 mL/min/1.73 m² | 47.6 | (0.26) | 42.3 | (0.25) |
| q1w | [26.0%] | (32.3-74.2) | [25.5%] | (29.0-65.3) |
| <20 mL/min/1.73 m² | 80.4 | (0.22) | 74.3 | (0.25) |
| q4d | [21.9%] | (51.5-95.9) | [25.8%] | (45.8-95.9) |
| <20 mL/min/1.73 m² | 82.6 | (0.31) | 73.5 | (0.31) |
| q1w | [31.3%] | (50.0-137.9) | [31.9%] | (44.4-123.9) |
| <20 mL/min/1.73 m² | 82.1 | (0.30) | 72.9 | (0.30) |
| q2w | [30.6%] | (50.3-132.8) | [31.0%] | (44.5-119.5) |

### Example 3: Pharmacokinetics of Migalastat in Fabry Patients with Renal Impairment

The computer modeling above provides scenarios for plasma migalastat exposure, but it does not account for renal impairment in Fabry patients. That is, the data does not include the pharmacodynamic component (plasma lyso-Gb3). Thus, two Fabry patients with renal impairment were evaluated. One patient (P1) had moderate renal impairment, while the other patient (P2) had severe renal impairment. Table 8 below shows plasma migalastat concentration for P1 compared with a phase 3 study by Amicus Therapeutics, Inc. (the FACETS study, Clinical Trial NCT00925301) and moderately impaired subjects from the renal impairment study of Example 1. There are two sets of migalastat concentration measurements taken 6 months apart, and the patient had been previously treated with migalastat. Table 9 shows similar information for P2, except compared with severely impaired patients from the renal impairment study of Example 1. The FACETS study was carried out in Fabry patients with amenable mutations where population PK was performed from sparse blood sampling. The comparison with the results from the FACETS study allows for comparison of PK in the Fabry population with mostly normal, but some mild and a few moderately impaired Fabry patients. None had severe renal impairment because these patients were excluded from the study.

TABLE 8

| | | | Migalastat | | Comparison | |
|---|---|---|---|---|---|---|
| | | | Conc 6 | Comparison | to Example | |
| Hour | Time | Migalastat | months later | to FACETS | 1 Moderate | |
| Nominal | (hr) | Conc (ng/mL) | (ng/mL) | PPK | Impairment | |
| 0 | Pre-dose | 19.9 | 36.4 | 8.70 | 64.5 | (105.6%) |
| 3 | 3 Hrs | 1620 | 2160 | 1180 (31.0%) | 1868 | (29.7%) |
| | Post | | | | | |
| 24 | 24 Hrs | 168 | 211 | — | 239 | (85.1%) |
| | Post | | | | | |
| 48 | 48 Hrs | 41.8 | 62.4 | 8.70 | 64.5 | (105.6%) |
| | Post | | | | | |

TABLE 9

| | | | | | Comparison |
|---|---|---|---|---|---|
| | | | Migalastat | Comparison | to Example |
| Hour | Time | Occa- | Concentration | to FACETS | 1 Severe |
| Nominal | Text | sion | (ng/mL) | PPK | Impairment |
| 2 | 2 h | 1 | 564 | — | 1549 (59.3%) |
| 48 | 48 h | 1 | 322 | 8.70 | 334 (38.2%) |
| 24 | 24 h | 2 | 569 | — | 770 (26.5%) |
| 48 | 48 h | 2 | 260 | 8.70 | 334 (38.2%) |

As seen from Table 8, C₄₈ₕ concentration, although increased by 49% over 6 months, remains similar to Example 1 non-Fabry subjects with moderate renal impairment. Cₘₐₓ has increased by 33% over 6 months, but remains similar to Example 1. C₂₄ₕ is similar to Example 1 for moderate renal impairment. eGFR_{MDRD remains within range for moderate impairment as well (}32 mL/min).

The percentages in parentheses are coefficients of variation, which are relatively high, corresponding to variability in the time 0h or time 48h concentrations. This result is likely due to the fact that half of the subjects from Example 1 with moderate renal impairment had low concentrations and half of them high concentrations.

The concentrations at 48 hours are higher than at 0 hours for P1 (third and fourth columns), but for a person with moderate impairment from Example 1, the concentration at 48 hours is the same as at 0 hours. This is because separate blood samples were taken at times 0 and 48 in P1. However, repeat dose modeling simulation outputs from single dose data were used in Example 1, therefore the values are one in the same.

Similar trends can be seen from Table 9. Accordingly, Tables 8 and 9 confirm similar pharmacokinetics of migalastat in Fabry and non-Fabry patients having similar renal impairment.

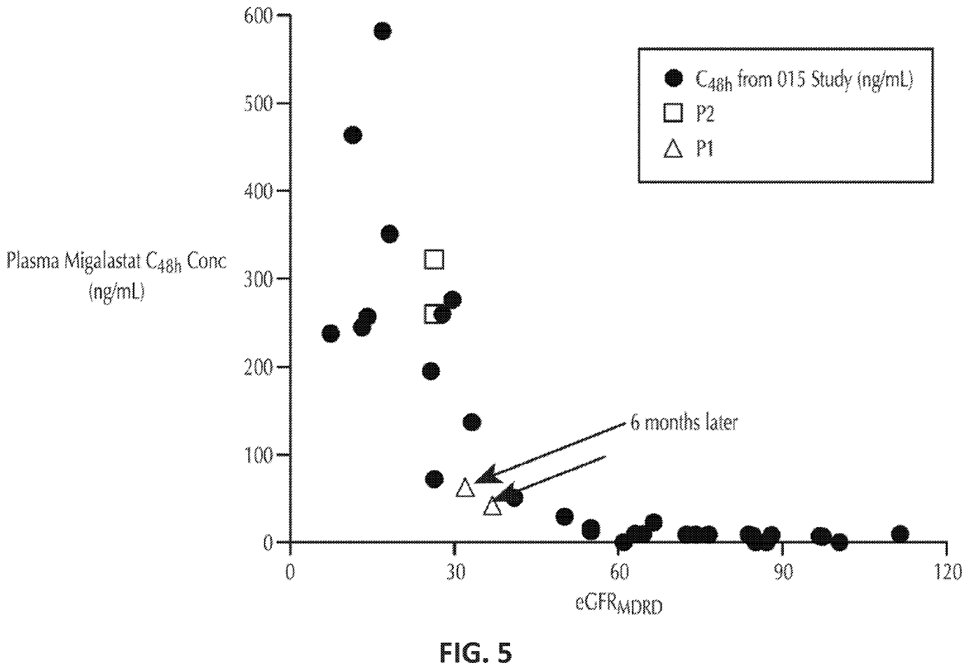

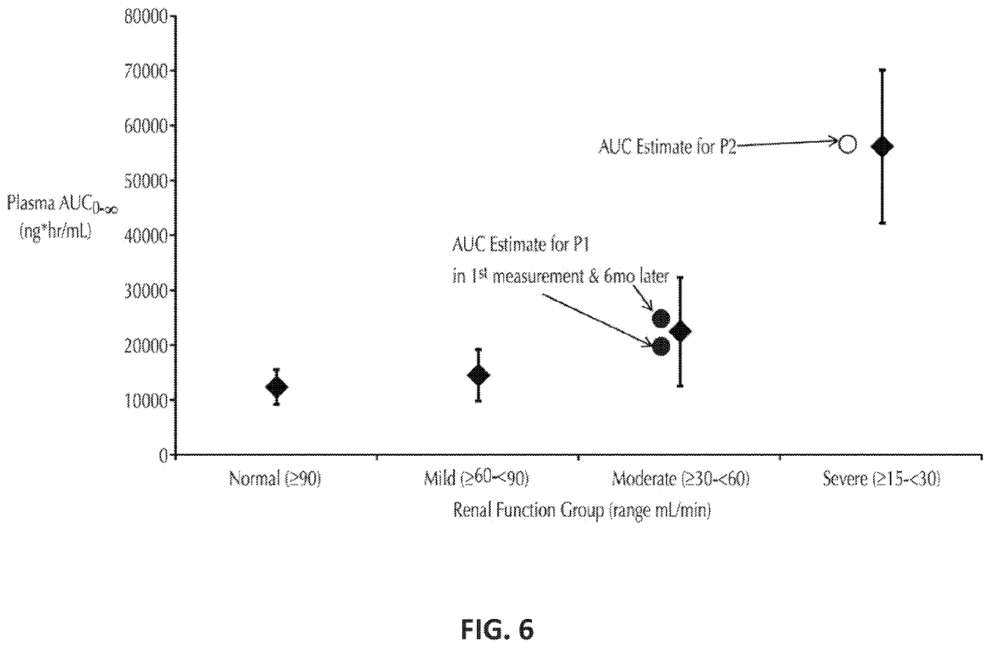

FIG. 5 shows the Fabry patients' plasma migalastat trough concentrations (C₄₈ₕ) versus the renal impairment study of Example 1. FIG. 6 shows the mean (SD) renal impairment study exposures versus Fabry patient estimated AUCs. As seen from the figure, P1 and P2 followed the general trend of the renal impairment study results in non-Fabry patients.

Table 10 below shows the Lyso-Gb3/eGFR for P1.

TABLE 10

| | Lyso-Gb3 | eGFR (MDRD), |
|---|---|---|
| Visits | (nM/L) | IDMS Traceable |
| 18 Month Visit | 11.1 | 42 |
| 24 Month Visit | 13.1 | 37 |
| 30 Month Visit | 10.8 | Unavailable |
| 34-Month Visit | 9.3 | 32 |

Despite continued decline in renal function to eGFR of 32 mL/min/1.73 m², plasma lyso-Gb3 has not shown clinically relevant changes from previous visits, and plasma migalastat concentrations remain similar to those observed in non-Fabry patients with moderate renal impairment.

This study demonstrates that the renal impairment and pharmacokinetic trends in Fabry patients correlates with the trends of non-Fabry patients. Thus, the computer modeling can be relied upon to select an appropriate dosing regimen (i.e., every 2, 4 or 7 days).

### Example 4: Additional Simulations on Renal Impairment Subjects

This example provides additional computer simulations of migalastat dosing of the renal impairment subjects of Example 1.

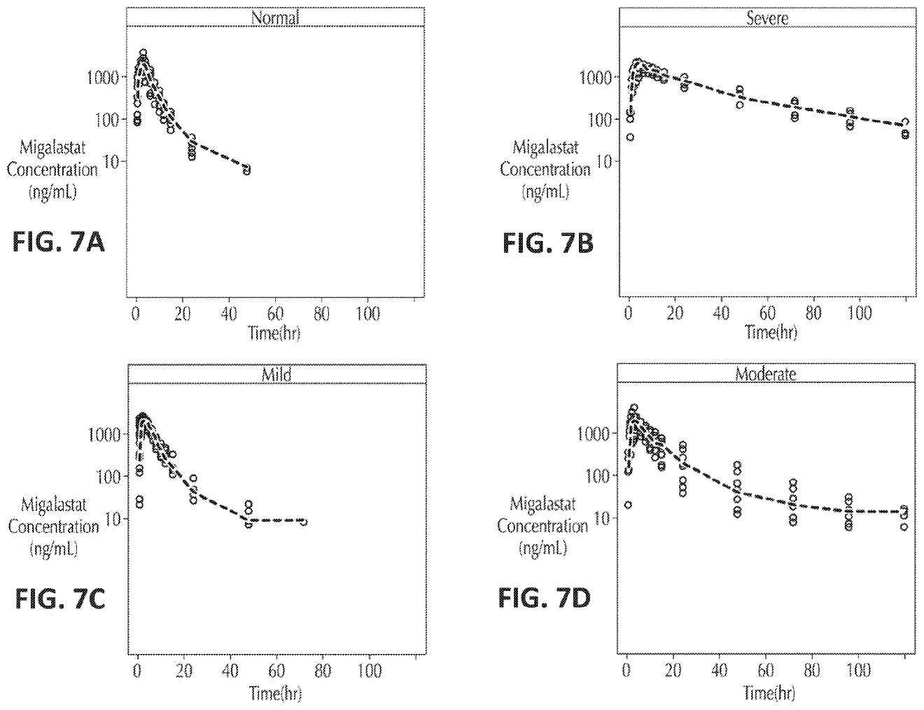

FIGS. 7A-D show simulated median and observed migalastat concentration versus time in normal, severe, mild and moderate renal impairment subjects, respectively. Table 11 below shows the data:

TABLE 11

| Renal Function Group | | | | |
|---|---|---|---|---|
| (CL_{CR} range | Cₘₐₓ ^{a} | AUC_{0-∞} ^{a} | AUC | |
| mL/min), N | (ng/ml) | (hr*ng/ml) | Ratio | t_{1/2} ^{c} (hr) |
| Normal (>=90), 8 | 2270 (37.6) | 12808 (31.3) | — | 6.2 (1.6) |
| Mild (>=60-<90), 8 | 2278 (22.5) | 15359 (25.2) | 1.2 | 8.0 (2.8) |
| Moderate | 2058 (47.1) | 23897 (38.9) | 1.9 | 23.0 (13.3) |
| (>=30-<60), 8 | | | | |
| Severe (<30), 4 | 2122 (29.1) | 61208 (23.1) | 4.8 | 32.5 (2.4) |

^{a} Geometric mean (CV %)
^{c} Mean (SD)

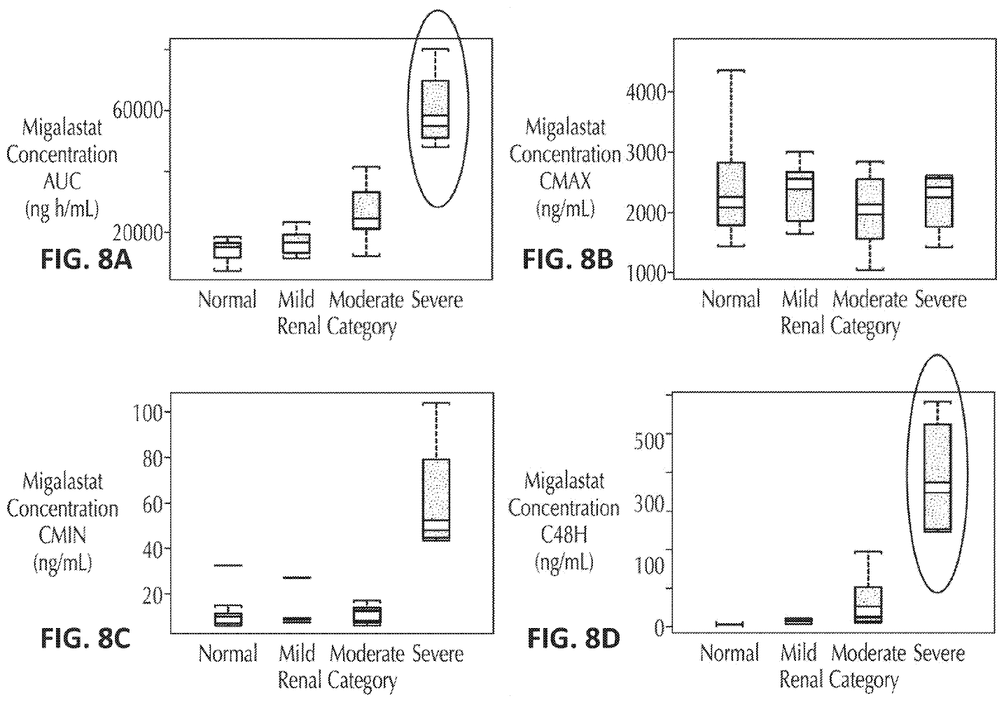

FIGS. 8A-D show Cₘₐₓ, AUC, Cₘᵢₙ and C₄₈ₕ, respectively, for normal, mild, moderate and severe renal impairment subjects.

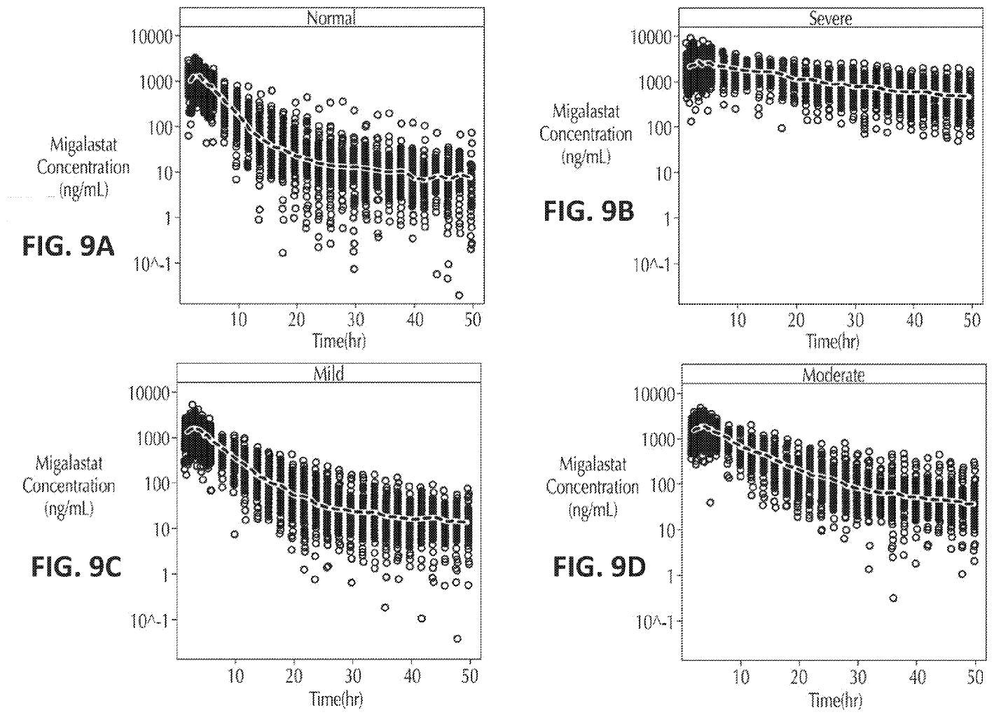

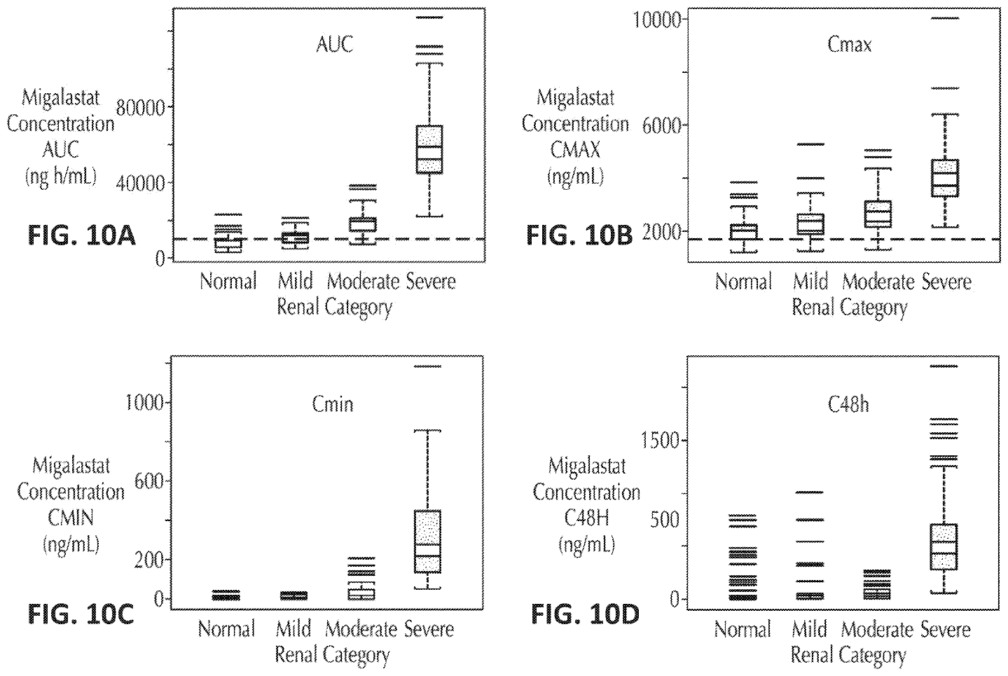

FIGS. 9A-D show the steady state prediction for QOD. The dashed line is the mean value from the QT study. FIGS. 10A-D show Cₘₐₓ, AUC, Cₘᵢₙ and C₄₈ₕ, respectively for the same simulation.

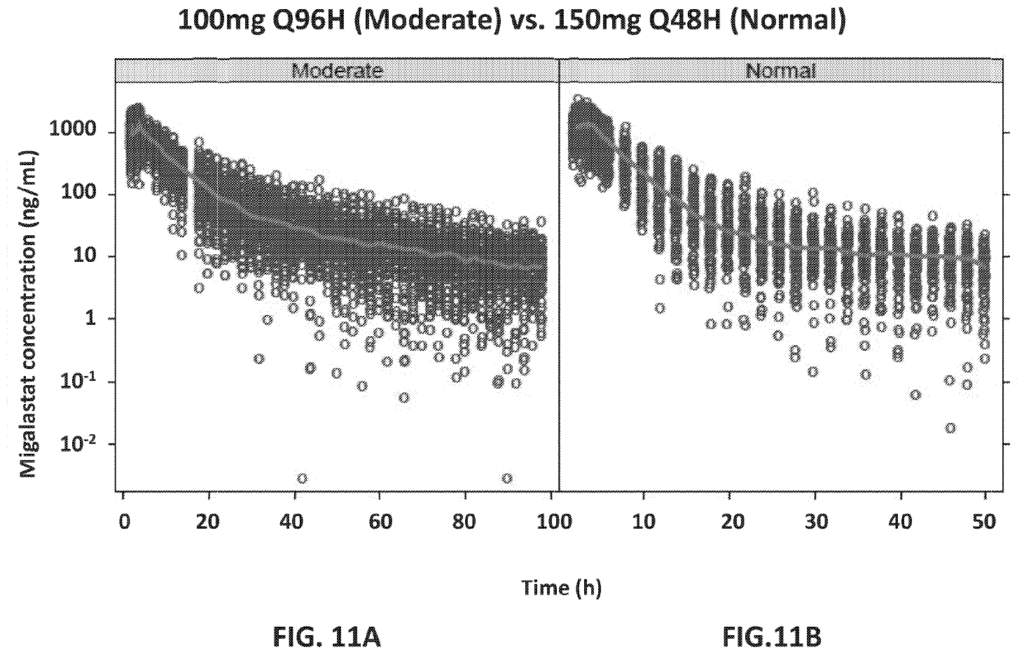

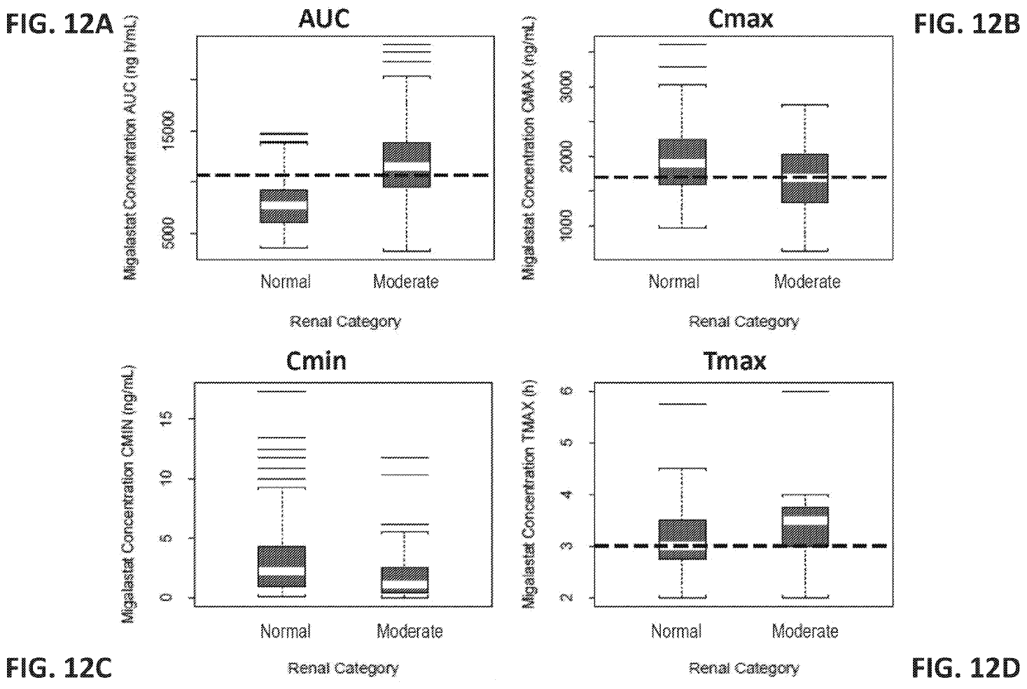

FIGS. 11A-B compare migalastat concentration after administration of 100 mg migalastat over 96 hours in a patient with moderate renal impairment to administration of 150 mg migalastat over 48 hours in a patient with normal kidney function. FIGS. 12A-D compare the Cₘₐₓ, AUC, Cₘᵢₙ and C₄₈ₕ, respectively, for the same simulation.

### Example 5: Proposed Study for Evaluation of Safety, Pharmacokinetics and Pharmacodynamics of Migalastat HCl in Fabry Patients with Amenable Mutations and Severe Renal Impairment

A study is proposed to evaluate the safety, pharmacokinetics and pharmacodynamics of migalastat HCl in Fabry subjects with amenable mutations and severe renal impairment (i.e., eGFR <30 mL/min/1.73 m²). Instead of lowering the dosage (i.e., less than 150 mg) a dose of 150 mg of migalastat HCl was maintained but administered less frequently. Subjects with eGFR_{MDRD} greater than or equal to 10 and less than 20 will receive the dose every 7 days (Q7D). Subjects with eGFR_{MDRD} greater than or equal to 20 and less than 30 will receive the dose every 4 days (Q4D). If a subject receiving the Q4D dose has a renal function which declines below 20 mL/min/1.73 m², the subject's dosing regimen is changed to Q7D. Any subject who begins dialysis treatment or undergoes renal transplantation will be discontinued from the study.

All subjects entering in this study will undergo screening (Visit 1) to confirm enrollment eligibility. Subjects who meet eligibility criteria will have a Baseline Visit (Visit 2) within 30 days of screening, including PK assessments. On-study visits will be scheduled every 3 months for a total of 12 months. Based on the PK/PD results from each site visit, as needed a follow-up visit or phone contact will be scheduled 1 month later. If PK/PD results indicate that a change in dose regimen is warranted, the subject will be advised to adjust the duration between doses and laboratory assessments will be done either locally or at the site.

Safety Analysis

There will be continuous monitoring of safety data and specific stopping criteria will be established for discontinuation of subjects who show evidence of declining renal function. Subjects with an eGFR <10 mL/min/1.73 m² on 2 consecutive visits will be discontinued from migalastat and withdrawn from the study.

Pharmacokinetic Sampling

Full PK blood sampling will be conducted at Visit 2 according to each subject's starting migalastat regimen. Subjects starting at a Q4D regimen will have PK assessments conducted predose and at 1, 2, 3, 4, 6, 8, 12, 24, 48, and 96 hours postdose. Subjects starting at a Q7D regimen will have PK assessments conducted predose and at 1, 2, 3, 4, 6, 8, 12, 24, 48, 96, and 168 hours postdose. At subsequent visits, subjects will undergo sparse sampling at 24, 48, and 96 hours postdose for subjects on Q4D regimen and at 24, 48, 96, and 168 hours for subjects on Q7D regimen.

At Visit 2, spot urine collections will be taken within 1 hour before dosing followed by a postdose total urine collection for the duration of each dosing interval at 0 to 4 hours, 4 to 8 hours, 8 to 12 hours, 12 to 24 hours, 24 to 48 hours, 48 to 72 hours, and 72 to 96 hours for subjects on a Q4D regimen. Collection intervals will be the same for subjects on a Q7D regimen with the addition of collections at 96 to 120 hours, 120 to 144 hours, and 144 to 168 hours.

For subjects with a dose regimen change, full PK blood and urine collections as detailed above will be done at the visit following the regimen change.

Dose Regimen Modifications

This protocol allows dose regimen changes on a subject-specific basis. Starting dose for each subject will be migalastat HCl 150 mg at a regimen based on eGFR, as noted above. For subjects who begin the study on a Q4D regimen, a decrease in eGFR to <20 mL/min/1.73 m² at 2 consecutive visits (including follow-up visits) automatically will trigger a switch to the Q7D regimen.

Plasma lyso-Gb3 will be monitored at each visit. If a subject has an increase in plasma lyso-Gb3 relative to their previous measurement, a confirmatory retest will take place. If the confirmatory value is also higher than their previous measurement, the average of the 2 values will be the subject's new reference value. If the retest is not higher than the subject's previous measurement, there will be no new reference value at that visit.

Throughout the study, stopping criteria will be applied on a per-subject basis. Subjects who have eGFR_{MDRD}<10 mL/min/1.73 m² for 2 consecutive visits or who undergo a dialysis or renal transplant will be discontinued from treatment. Subjects may also be discontinued from treatment at the discretion of the investigator and a medical monitor.

Duration of Study Treatment

Following a screening period of up to 30 days, enrolled subjects will receive migalastat treatment for 12 months. At the end of 12 months, subjects may be eligible to enroll in a separate open-label extension study.

Criteria for Evaluation

Safety: Safety parameters include physical examinations, vital signs (blood pressure, heart rate, respiratory rate, and body temperature), 12-lead electrocardiograms, clinical laboratory parameters (serum chemistry, hematology, and urinalysis), eGFR, and adverse events.

PK: The following PK parameters will be calculated, if available, based on the plasma concentrations of migalastat: maximum observed concentration (Cₘₐₓ), concentration at the end of a dosing interval at steady state (C_{trough}), average plasma migalastat concentration over the dosing interval (C_{avg}), time to maximum concentration (tₘₐₓ), apparent terminal elimination half-life (t_{1/2}), area under the concentration-time curve from time zero to the last measurable concentration (AUC₀₋ₜ) and extrapolated to infinity (AUC_{0-∞}), and plasma clearance (CL/F).

The following PK parameters will be calculated based on urine migalastat concentrations: total amount excreted over the dosing interval (Ae₀₋ₜ), fraction of the dose recovered in urine over the dosing interval (Fe), and renal clearance (CLr).

PD: PD parameters include plasma lyso-Gb3, eGFR_{MDRD}, and eGFR_{CKD-EPI}.

Statistical Methods

Plasma migalastat concentrations from serial PK blood and urine sampling will be determined by noncompartmental analysis using Phoenix®-WinNonlin® software, version 7.0 or higher. Plasma migalastat sparse PK blood sampling will be analyzed by a Population PK model. The Population PK model will assess and validate severe renal impairment dose regimen simulations, and will be provided as a separate report. PK/PD modeling may be explored.

Continuous PD and safety data will be summarized using descriptive statistics (number, mean, median, minimum, and maximum). Categorical variables will be presented by number (%).

### Example 6: Pharmacokinetics of Migalastat HCl in a Fabry Patient with Severe Renal Impairment

A patient (P3) was enrolled in a previous migalastat study, but discontinued in May 2016 as a result of sever renal impairment (i.e. eGFR <30 mL/min/1.73 m²). Beginning in May 2017, P3 was dosed with migalastat HCl Q4D. PK data was collected every three months. Table 12 shows P3's PK data compared to patients dosed QOD with varying levels of renal function.

TABLE 12
QOD Regimen PK in AT1001-015, -011 vs Q4D Regimen in Severe RI Patient 2004-5029

| Renal Function | | | | | | | | |
|---|---|---|---|---|---|---|---|---|
| Group (CL_{cr} | Cₘₐₓ^{a} | | | | | | AUC₀₋ₜ^{a} | |
| range mL/min), N | (ng/mL) | tₘₐₓ^{b} (hr) | C_{48 h}^{c} (ng/mL) | | C_{96 h}^{c} (ng/mL) | | (hr*ng/mL) | |
| Normal (≥90), 8 | 2100 (26.1) | 2.5 (1.5-3.0) | 5.70 | (18.9) | BLQ | | 12299 | (25.0) |
| Mild (≥60-<90), 8 | 2191 (27.5) | 2.5 (1.5-4.0) | 9.34 | (62.1) | BLQ | | 14374 | (31.1) |
| Moderate (≥30-<60), 8 | 1868 (29.7) | 3.0 (1.5-4.0) | 64.5 | (105.6) | 16.7 | (65.3) | 22122 | (41.4) |
| Severe (≥15-<30), 8 | 2078 (43.3) | 4.0 (3.0-8.0) | 334 | (37.7) | 105 | (37.4) | 53098 | (25.3) |

| | | | | | | | |
|---|---|---|---|---|---|---|---|
| 011 PPK (≥30-≥90), 62 | 1180 (32.9) | 3.0 (2.0-4.5) | 7.54 | (84.3) | — | 9033 | (35.1) |

| | | | | | |
|---|---|---|---|---|---|
| 2004-5029 (21), 5/17 | 2250 | 2.0 | 77.4 | 18.4 | 31870 |
| 2004-5029 (19), 8/17 | N/A | N/A | 130 | 61.3 | N/A |
| 2004-5029 (15), 11/17 | N/A | N/A | 401 | 86.2 | N/A |

| Renal Function | | | | | |
|---|---|---|---|---|---|
| Group (CL_{cr} | AUC_{0-∞}^{a} | | | CL/F^{c} | |
| range mL/min), N | (hr*ng/mL) | t_{1/2}^{c} (hr) | | (L/hr) | Fᵣₑₗ |
| Normal (≥90), 8 | 12389 (24.8) | 6.4 | (29.7) | 12.5 (30.4) | — |
| Mild (≥60-<90), 8 | 14520 (30.8) | 7.7 | (39.0) | 10.7 (29.0) | 1.2 |
| Moderate (≥30-<60), 8 | 22455 (41.2) | 22.2 | (64.0) | 7.16 (39.1) | 1.8 |
| Severe (≥15-<30), 8 | 56174 (24.2) | 32.3 | (22.9) | 2.74 (25.2) | 4.5 |
| 011 PPK (≥30-≥90), 62 | — | 20.6 | (19.2) | 17.1 (54.8) | — |

| | | | | |
|---|---|---|---|---|
| 2004-5029 (21), 5/17 | 32101 | 13.2 | 4.67 | 2.6 |
| 2004-5029 (19), 8/17 | N/A | N/A | N/A | N/A |
| 2004-5029 (15), 11/17 | N/A | N/A | N/A | N/A |

^{a}Geometric mean (CV %)
^{b}Median (min-max)
^{c}Mean (CV %)
N/A = not available

Table 13 shows the plasma concentration of migalastat for P3 after dosing at various time points.

TABLE 13

| | Plasma Migalastat Conc (ng/mL) | | | | | |
|---|---|---|---|---|---|---|
| Time (h) | Mar ′11 | Feb ′15 | Aug ′15 | May ′17 | Aug ′17 | Nov ′17 |
| 0 | 38.1 | 19.9 | 36.4 | BLQ | — | — |
| 3 | 1560 | 1620 | 2160 | 2110 | — | — |
| 4 | 1570 | — | — | 1970 | — | — |
| 8 | 802 | — | — | 1380 | — | — |
| 24 | — | 168 | 211 | 358 | — | — |
| 48 | — | 41.8 | 62.4 | 77.4 | 130 | 401 |
| 96 | — | — | — | 18.4 | 61.3 | 86.2 |
| eGFR(mL/min/1.73 m²) | 59 | N/A | 32 | 21 | 19 | 15 |

Table 14 shows the Lyso-Gb3 and eGFR of P3 over time.

TABLE 14

| | Migalastat | | |
|---|---|---|---|
| | Dosing | Lyso-Gb3 | eGFR(MDRD), |
| Visit Date | Frequency | (nmol/L) | IDMS Traceable |
| September 2010 | QOD | 27.30 | 54 |
| March 2011 | QOD | 24.15 | 59 |
| February 2014 | QOD | 11.10 | 42 |
| August 2014 | QOD | 13.10 | 37 |
| February 2015 | QOD | 10.80 | — |
| June 2015 | QOD | 9.30 | 32 |
| September 2015 | QOD | 7.36 | 33 |
| March 2016 | QOD | 8.46 | 28 |
| May 2017 | Q4D | 13.10 | 21 |
| August 2017 | Q4D | 9.85 | 19 |
| November 2017 | Q4D | 8.55 | 15 |
| January 2018 | Q7D | 9.04 | 13 |
| February 2018 | Q7D | 7.04 | 13 |

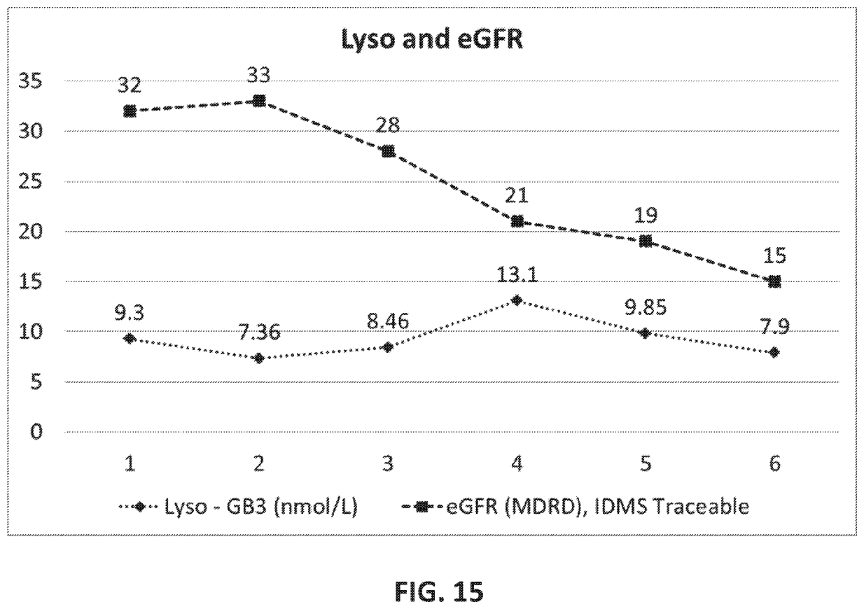

FIG. 15 shows the Lyso-Gb3 and eGFR of P3 over time.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for the treatment of Fabry disease in a patient having renal impairment, the method comprising administering to the patient about 100 mg to about 300 mg free base equivalent (FBE) of migalastat or salt thereof at a frequency of once every four days.

2. The method of claim 1, wherein the patient has moderate renal impairment.

3. The method of claim 1, wherein the patient has severe renal impairment.

4. The method of claim 1, wherein the patient has a HEK assay amenable mutation in α-galactosidase A.

5. The method of claim 1, wherein the migalastat is in a solid dosage form.

6. The method of claim 1, wherein the patient is administered about 123 mg FBE.

7. The method of claim 1, wherein the patient is administered about 150 mg of migalastat HCl.

8. The method of claim 1, wherein the migalastat is administered orally.

9. A method for the treatment of Fabry disease in a patient having renal impairment, the method comprising administering to the patient about 100 mg to about 300 mg free base equivalent (FBE) of migalastat or salt thereof at a frequency of once every seven days.

10. The method of claim 9, wherein the patient has moderate renal impairment.

11. The method of claim 9, wherein the patient has severe renal impairment.

12. The method of claim 9, wherein the patient has a HEK assay amenable mutation in α-galactosidase A.

13. The method of claim 9, wherein the migalastat is in a solid dosage form.

14. The method of claim 9, wherein the patient is administered about 123 mg FBE.

15. The method of claim 9, wherein the patient is administered about 150 mg of migalastat HCl.

16. The method of claim 9, wherein the migalastat is administered orally.

17. A method for the treatment of Fabry disease in a patient having renal impairment, the method comprising:
administering to the patient about 100 mg to about 300 mg free base equivalent (FBE) of migalastat or salt thereof at a first frequency of once every other day for a first time period; and
administering to the patient about 100 mg to about 300 mg FBE of migalastat or salt thereof at a second frequency of less than once every other day for a second time period.

18. The method of claim 17, wherein the second frequency is in a range of once every three days to once every seven days.

19. The method of claim 17, wherein the second frequency is once every four or once every seven days.

20. The method of claim 17, wherein administration at the second frequency begins after a reduction in the patient's estimated glomerular filtration rate (eGFR).

21-50. (canceled)


SEQUENCE LISTING (2 sequences)

SEQ ID NO: 1 - DNA, 12436 bp, Homo sapiens
cccttctgtaggggcagagaggttctacttcattactgcgtctcctgggaaggccatcaggactgctggctaaagtgggaaccaggactctttgtgagttaagaatttgtgtatttatatgtgtgttatacacattttttaaaaaactgtaacgacatcaggttgagcagtcgtctccgggtggtgaattatgtgtatttttaaattttatactatattgttatttttcaaatgttcgaaattgaatatgtagattgttgttatcagcagaaaaataaacattattcaaatactctattcagtaaagtaatttattgggcgcctttgtcaagcacgcatttgcctagatgtgactctacagataaaattcacttggggcctccccttacagacaatcaggcagtggagactgagtgcctgaatggatagaccagcactcagaccactattttcagtatctgtttttcttaactcagggccgtggttttcaaacgtttttcgccttacggtcacccttagggtcccccgagaccggcccagacagacagatatacaaaaacacatacacagtcatgagcgtccaccatttccccaccaggcgcagcacaggcggcttcccggcactgagatgggggggaggagggagagagcgcgaggggggaggggaaagcagagaacgaaagaggcggaggcggcccccgaaccccgctctggtcttcatcatcaccacccctgggtccccagttcccacccacacaccaacctctaacgataccgggtaattttcctccttcttccctcaaacggctatagcgagacggtagacgacgaccagaactacttctgctcacgtaagcgagtaatcacgtgagcgcctacgtcatgtgagatctcggtcacgtgagcaactctcggcttaaactcgggatcactaaggtgccgcacttccttctggtatggaaatagggcgggtcaatatcaagaaaggaagagggtgattggttagcggaacgtcttacgtgactgattattggtctacctctggggataaccgtcccagttgccagagaaacaataacgtcattatttaataagtcatcggtgattggtccgcccctgaggttaatcttaaaagcccaggttacccgcggaaatttatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcaggtatcagatattgggtactcccttccctttgcttttccatgtgtttgggtgtgtttggggaactggagagtctcaacgggaacagttgagcccgagggagagctcccccacccgactctgctgctgcttttttatccccagcaaactgtcccgaatcaggactagccctaaactttctctgtgtgacctttcctgggatgggagtccggccagcggcccctgtttctttctctctctctctctctctcgttctccttctctttctctttctcttctttcctctctctttctctctctccctgcccggttctcttttttcactgctccttgcagagcagggccaccccataggcagtgtgcccaaagtagccctgcccggttctattcagacccttcttgtgaacttctgctcttcctctgccgggtgctaaccgttagaacatctagggtgggtaggaggaatggggaactaagattcgtgccattttttctccttttggggtcgtggatttctcggcagtatctcgagggagttagagagaccataaggtcgctgagatctctcccacctcgcccatgagcgtggcatcaggctggaaggttgacatggaggaactttatacatttacacctttgcgtgagggttgaggctggattagataggtattgaacatatctgaccctcacaatccttatctgtaaattgggattacaaccttttaatttcagggagctgacaaaaaaaatctgaaaaatagttcttatctcacacaggtgagttttcaaggagataacctatttaaagtacatagcacagcgcttgaccattcaactgcgcttacagagcaaatgttcaatgggaaaatgaatgtaaatctacaaatctgaatgaatatgtgtatttttctggagagaggatatttacctttcttcaaattctcaaagggctctgtgatttaaaaaaggttaggaatcactgatagatgttggtaaaaggtggcagtcacagtacatttctgtgtccataagttattcctatgaatatctttatagataaagtcaggatgttggtcagacatcacagaagaaattggccttgtaagtttcatgtgaccctgtggtacagtatgtgtggcaattttgcccatcacggatttttttttattggtatttgcatctgattataaaactaatgcatgatcattgcaaaaaatgtagataaagaagagcaaaatgaaaataaagatttccccccaccgttccaccacccagaaataatcatggtttaaatgttaatatacaaccttacaattgttttctatataaatgaaaacatagatttctttatttcattattttccataaaaaatggatcatgtttatgtcatgtttggctaatggcaagaccctggcacccagtctgggctcaaattctgcctcattgttacttagccctgtgacattgggtaaattacactttttttttttttttttttttgagacggggtctcgctctgtcgcccaggctggagtgcagtggcacgatctcggctcactgcaagtccgcctcctgggttcacgccattcttctgcctcagcctcccgagtagctgggactacaggcgcctgccaccacgcctggctctttttttttttttttttttttttagtacagacggggtttcaccatgttagccagggtggtctcaatctcctgacctcgtgattcgcccgcctcagcctcccaaagtgctggtgtgagccaccgtgcccagccttacttttttttttgagagggggtctcactctgtcacccaggttggagtgcagtggcgcgatctctgctcagtgcaaactccacctcccgggtttaagcagttctcctgtcgtagtctcctgagtagctgggattacaggcacaccaccacggccagctaatttttgtattttcagtagagacgggtttcaccatgttgcccaagctggtctcgaactcctggcctcaagtgatctgcccgccttggcctcccagagtgctgggattacaggtgtgagccaccgcacccggcctcttttttcttttttagtctatcataccttgcaaatacagtggttcttcctatgtgttggttttgatatttatgtaatcaaacacatcagtttttcctttctgatttctgactttggggtcatgctgagaaagtcctttcctacctgaagataatacagtatatacgtttcttactagtatttttgtggatttttaaaatatttaaatctttagtccatctgaacttgttcttctatcagaaatgccacatttaataaataataagtcccatggtatcagatggctggaaggacctctttcgaaactttgtttaattccattaatctgtgtattcttattctaatgctaatagttccacactagcttcctttatcttttttttctttttttttttttttttgagctggagtttcgctcttgttgcccaggctggagtacaatgtcacgatctcggttcaccgcaacctccgcctcccaggttcaagcaattctcctgcctcatcctcgcgagtagctggaattacaggcatgcgccaccacgcctagctattttgtatttttagtagagatggggtttctccatgttggtcaggctggtctcaaactcccagcctcaggtgatctgcctgcctcggcctcccaaaatgctgttattacaggcgtgagccaccacgcccagccttcatcttttaatgaatgtacatgtatgtaatcttttaggtgaactttttgtaatgttgtgccaagttccttaaaaagcccttttggaagctgggcaggtggccacgcctgtaatcccagcattttgggagtctgaggcaggtggatcacttgaggccaggagttcaagactagcctagccaaaatgcaaaaccctgtctctactaaagatacaaaaattagccggatgcgatggcacatgcctgtaatctcagctactcgggaggctgaggtagaagaatcgcttgaaccggggaggcagaggttgcagtgagcaagatggcgccactgcactccagcctgggtgacagagggagactccatctcaaaaaaaaaaaaaaaaaaaaagataaaaaggaaacctaagtactcttgggctttgttaaggattttgttaaatatacaaaggattgcagggaaaattaacttatttttaatattgagtatgcttatccaagagcaaaataatatttctccatttattcaaatcatttaggagcatcatagttttaacatatgggccttgcacgtatcttaaatttatctctaggcattttaggttgttcagttgttcttgtgaatgggatctttttctccaaataggattattgttgatatctgttgattatgttaactttgtagtttctgactttactgaactgtcttcttagatctaatactcttttcaatttcatcatatatttctcattcctattttgtttggggtttttagggcgggaatattaacgggataagagagacaaaagaaaatctggaaaaacaattcattttaccttacattgcttgtgattactaccacactattactgggttggaaaaaattgtgaaatcccaaggtgcctaataaatgggaggtacctaagtgttcatttaatgaattgtaatgattattggaatttctctttcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgtgagtttatagataatgttcttgttcattcagaggactgtaagcacttctgtacagaagcttgtttagaaacagccctcatggccgggcgtggtggctcacgctgtaatcccaacactttgggaggccgaggcgggtggatcacctgaggtcaagagttcaagaccagcctggccaacatggtgaaaccccaactctattaaaagtacaaaaaattagctgggcatggtggtgaacgcctgtaaccccagctacttgggaggctgaggcaggagaatcgcttgaacccaggaggtggaagtttcagtgagctgagatcacgccattgcactctagcctgggcaacaaaagagaaactccatctcaaaaaaaaaaacaaggaaaaaaagaaacagccctcatgacacttagaaagtagaatagctggctgttatctgaacattgaattgtaaggcttatcaggtggactttgcattccatcagcagacaatttttttttttttttttttttgagatggagtctcattctgtctcccaggctggagggcagtggtgcgatctcggctcactgcaagctccacctcctgggttcatgccattctcctgcctcagcctcccaagtagctgggaccacaggcacccgccaccatgcccagttaattttttgtatttttagtagagacggggtttcaccatgttagccaagatggtctcgatctcctgacctcgtgatccgcccacctcggcctcccaaagtgctgggattacaggcatgagccaccgcgcctagcctacaaatgttttgtaatagctcttgaggcccatcttggagttctccttttgctaaaaccactgaactctctaggaggaaaaaggaacttggttcttgacatatgtgtgcatgtatttccatataacctttaggaagctattgcaatggtactataaactagaattttagaagatagaaggaaaatattctggagatcattgaagagaaatggagtccaacactagttaaagatgatgaagacagatttttttttttgacggagtctcgctctgtcgcccaggctggagtgcagtggcacaatctcagctcactgcaaccctccacctcttgggttcaagtgattctcctgcctcagcctcccaagtagctgggactacaggcgcacaccaccacgcccggctaatttttgtatttttagtagagacaaggtttcaccatattcgccaggctggtctcgaactcctgaccttgtaatccgcccaccttggcctcccaaagtgctgggattacaggcatgagccaccacgcccggccgatgaagacagattttattcagtactaccacagtagaggaaagagccaagttcaattccaaatacaacaaagacaggtggagatttatagccaatgagcagattgagggggtcagtggatggaatatttaagaagacatcaagggtagggagcttcttgctaaagcttcatgtacttaaacaagaagggtgggggatgagggaaattgatcagatatcaatggtggcagtattgacttagcaggattcttgctaagaggtcttgctaggacagacataggaagccaaggtggaggtctagtcgaaaagaaggctcatcagagaagtctaactaaagtttggtcaagaagagtctttgtcaaggtaaatctatcatttccctcaaaaggtaattttcaggatcccatcaggaagattagcatggctgctagctttctcctcagttctgggctatagctcacatgcctagtttgaactagctcagcagaactgggggatttattctttgtcttccaacaaactcatctggatgattttgggggtttgtggggaaaagcccccaatacctggtgaagtaaccttgtctcttcccccagcctggaatggttctctctttctgctacctcacgattgtgcttctacaatggtgactcttttcctccctctcatttcaggttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggtaatgtttcattccagagatttagccacaaaggaaagaactttgaggccatggtagctgagccaaagaaccaatcttcagaattttaaataccctgtcacaatactggaaataattattctccatgtgccagagctcccatctcttctctttcagttcattaattaattaattaattcatgtaaaatccatgcatacctaaccatagctaatattgtgcacttataattcaagagggctctaagagttaattagtaattgtaactctctataacatcatttaggggagtccaggttgtcaatcggtcacagagaaagaagcatcttcattcctgcctttcctcaatatacacaccatctctgcactacttcctcagaacaatcccagcagtctgggaggtactttacacaatttaagcacagagcaactgcctgtccctgctgctagtttaaacatgaaccttccaggtagcctcttcttaaaatatacagccccagctgggcatgatggctcatgcctgtaatcctagcactttgggaggctgaggcgggtggattacttgaggtcaggagttcgagaccaccctggccaacatggtgaaaccccatctctagtaaaaatacaaaaattagctgactttggtggcacatgcctgtaatcccagctacttgggaagctgagacagaagagtcacttgaacctgggaaacagaggttgcagtgagccaagatcgcaccactgcactccaccctggatgacagactgaaccccatctcaaaaaattaaaataaaataaaataaaataactatatatatagccccagctggaaattcatttctttcccttattttacccattgttttctcatacaggttataagcacatgtccttggccctgaataggactggcagaagcattgtgtactcctgtgagtggcctctttatatgtggccctttcaaaaggtgagatagtgagcccagaatccaatagaactgtactgatagatagaacttgacaacaaaggaaaccaaggtctccttcaaagtccaacgttacttactatcatcctaccatctctcccaggttccaaccacttctcaccatccccactgctgtaattatagcctaagctaccatcacctggaaagtcatccttgtgtcttcccctttatttcaccattcatgtcctgtctatcaacagtccttccaccagtatctctaaaatatctcctgaatcagcccacttccttccatcttcactacatgcaccctggccttccaagctactatcggctctcaaccagactgctgggaccacctgatctctctgcttccactctgtctcaacccccatctattttccaagcagcactagagttatcatattaaaatgtaaatatcagttttttttttaaagaaaaaaaccctgagacttaacagagttataaaaaatataaatgtcatcatcagttccctgcttaaaacccttaactcgcttccaattgcacttggaatgaaaccaaactgcactgatccagcccttgcctgcctccccaaagtccaaggggtcatggctctttccctggctacactggttttctttctgtccctcaacactgcaagcctattgctgccccagggcctttacacttgctttttttctgcctagaacagttcttccccaaagatttttaaagggccgggctccttaacattgaagtcgcagaccaaacgccacatatgcagacagttcttctctaactactttaaaatagccctctgtccattcattcttcatcacattaacctgtttaattttcttctcagagctccacactatttggaagtatttgttgacttgttaccatgtctccccactagagtgtaagtttcatgagggcagggaccttgtctgactttgactgtatctctcgcatatggttaagtgttaaatagttatttatggaatgaatccctattattccctcattatctctgcaaaatagtcttttttctcaacatcttaaacctgatatcccacctgcctatctacaaactttttttttgcgacagagtctcactgtcacccaggctagagtgcagtggcgccatctcggctcactgcaacctccgcctcccgggtttaagcgattctcttgcctcagcctcccagtagctgggattataggcgtgcgctaccacatctggctaatttttgtatttttagtagagatggtttcaccatgttggccaggcttgtctcgaactcctgacctcagatgatccacctgcctcggcctcccaaagtgctgggattacaggcatgagccaccgtgcccagcctctacaaactttttattccattaacaaactatatgctgggatttaagttttcttaatacttgatggagtcctatgtaattttcgagcttttaattttactaagaccattttagttctgattatagaagtaaattaactttaagggatttcaagttatatggcctacttctgaagcaaacttcttacagtgaaaattcattataagggtttagacctccttatggagacgttcaatctgtaaactcaagagaaggctacaagtgcctcctttaaactgttttcatctcacaaggatgttagtagaaagtaaacagaagagtcatatctgttttcacagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatggtaaaaacttgagccctccttgttcaagaccctgcggtaggcttgtttcctattttgacattcaaggtaaatacaggtaaagttcctgggaggaggctttatgtgagagtacttagagcaggatgctgtggaaagtggtttctccatatgggtcatctaggtaactttaagaatgtttcctcctctcttgtttgaattatttcattctttttctcagttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacaggtaaataagagtatatattttaagatggctttatatacccaataccaactttgtcttgggcctaaatctatttttttcccttgctcttgatgttactatcagtaataaagcttcttgctagaaacattactttatttccaaaataatgctacaggatcattttaatttttcctacaagtgcttgatagttctgacattaagaatgaatgccaaactaacagggccacttatcactagttgctaagcaaccacactttcttggtttttcagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtttattttattgccaactactacttcctgtccacctttttctccattcactttaaaagctcaaggctaggtggctcatgcctgtaatcccagcactttgggaggctgaggcgggcagatcacctgaggtcgggactttgagacccgcctggacaacatggtgaaaccccatttctaataaaaatataaaaattagccaggtgtggtggcgcacctgtggtcccagctactctgggggctgaggcatgagaatcgcttgaacccgggagtggaggttgcattgagctgagatcatgccacctcactccagcctgggcaacaaagattccatctcaaaaaaaaaaaaaaagccaggcacagtggctcatgcctggaatcccagcacttttggaagctgaggcaggcagatcacttgaggttaggatttcaagaccagcctggctaacatagtaaagccctgtctctactaaaaatacaaaaattagccaggtatggtggcgagcttctgtagccccagctactcaggagactgaggcaggagaatcacttgaacccgggaagtgggggggtgcagtgacccaagatcacgccactgcattccagcctgggcaacagagcaagactccatctcaaaaaaaaaagttctatttccttgaataaaattttccgaagtttaaactttaggaataaaactattaaacccgtatttactcatccagatacccaccccccttgttgagattctctcccaattatcaaaatgtgtagcatatttaactaccaagagctaaacatcattaagactgaaatgtattaagaaggatgtataggccaggcacggtgtctcacgcctgtaatcccaacactttgggaggccaagtcgggcggatcacgaggtcaggagatggagaccatcctggccaacatggtgaaaccccctctctactaaaaatacaaaaattagccaggcaggtggcaggcacctgtaatcccagctactccagaggctgaggcaggacaatcacttgaacctgggaggcagaggctgcagtgagctgaggttgtaccaattgcactccagcctaggtaacgagcaacactccatctcaaaaaaagaaaaaaaaaaagatgtataatttggaactgttaagaggcattttaaaga

SEQ ID NO: 2 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL